(19) United States
(12) Patent Application Publication
(10) Pub. No.: US 2013/0177516 A1
(43) Pub. Date: Jul. 11, 2013

(54) Cosmetic For Hair Containing Sugar Alcohol-Modified Silicone

(71) Applicants: Seiki Tamura, Ichihara-shi (JP); Tomohiro Iimura, Sodegaura-shi (JP); Tatsuo Souda, Ichihara-shi (JP); Haruhiko Furukawa, Chiba-shi (JP)

(72) Inventors: Seiki Tamura, Ichihara-shi (JP); Tomohiro Iimura, Sodegaura-shi (JP); Tatsuo Souda, Ichihara-shi (JP); Haruhiko Furukawa, Chiba-shi (JP)

(73) Assignee: DOW CORNING TORAY CO., LTD., Tokyo (JP)

(21) Appl. No.: 13/812,776

(22) PCT Filed: Jul. 28, 2011
(86) PCT No.: PCT/JP2011/067811

§ 371 Date: Mar. 26, 2013

(30) Foreign Application Priority Data
Jul. 30, 2010 (JP) 2010-173094

(51) Int. Cl. A61K 8/893; A61Q 5/00

(52) U.S. Cl. CPC A61K 8/893; A61Q 5/00
USPC 424/70.12; 556/445; 556/434; 8/405; 8/406; 8/407

(57) ABSTRACT

The present invention provides a cosmetic for hair which is superior in view of a sensation during use, effects of styling and/or conditioning hair after use, and cleansing properties. A sugar alcohol-modified silicone having a specified chemical structure in which a sugar alcohol-modified group and optionally a silylalkyl group having a dendron structure, and/or a long chain hydrocarbon group are possessed in one molecule is blended in a cosmetic for hair.

### TECHNICAL FIELD

The present invention relates to a cosmetic for hair comprising an organopolysiloxane modified with a sugar alcohol.

Priority is claimed on Japanese Patent Application No. 2010-173094, filed on Jul. 30, 2010, the content of which is incorporated herein by reference.

### BACKGROUND ART

Heretofore, as modified silicones to be blended in cosmetics, various materials have been known. For example, polyether-modified silicones have been widely used as surfactants. In addition, glycerol-modified silicones and sugar-modified silicone compounds are also reported, and blending these into cosmetics for hair is proposed.

For example, JP-A-2002-179798 and JP-A-2004-231607 disclose cosmetics for hair comprising (poly)glycerol-modified silicones. It is described therein that the aforementioned (poly)glycerol-modified silicones are superior as an emulsifier.

In addition, JP-A-2005-91752 and JP-A-2006-265339 disclose cosmetics for hair comprising branched polyglycerol-modified silicones, and describe that superior effects of protecting, repairing, modifying, and styling hair are exhibited, and a good sensation during use is provided.

### DISCLOSURE OF INVENTION

### Technical Problems

Cosmetics for hair comprising (poly)glycerol-modified silicones or branched polyglycerol-modified silicones need further improvements in view of a sensation during use such as a non-sticky sensation, smoothness or the like; effects of styling and/or conditioning hair after use such as set-retention ability, the feeling on touch of hair after drying or the like; and cleansing properties such as good foaming properties, foam quality and the like.

The present invention has been made in view of the circumstances of the aforementioned prior art. An objective of the present invention is to provide a cosmetic for hair in which a superior sensation during use, superior effects of styling and/or conditioning hair after use, and/or superior cleansing properties are exhibited. Namely, the objective of the present invention is to provide a cosmetic composition for hair in which smooth combability with fingers without exhibiting a frictional sensation during cleansing and applying to hair, namely during wetting, can be exhibited, the aforementioned effects are not lost by a rinsing operation, smooth combability with a comb or fingers during and after drying the hair is exhibited, a moisturizing feeling on touch is exhibited without uncomfortable stickiness, and/or a flexible styling sensation is provided to the hair. In addition, another objective of the present invention is to provide a cosmetic for hair in which the aforementioned various effects can be maintained.

Organopolysiloxanes modified with sugars as hydrophilic groups have been proposed, and it is described that they are used as a surfactant (JP-A-2002-119840 and JP-A-2008-274241). In addition, for example, JP-A-H05-186595 describes that sugar-modified silicones can be widely applied to various fields such as cosmetics, toiletry products, coating materials, plastic additives and the like. However, it is not disclosed or suggested that sugar alcohol-modified silicones possessing specified structures are blended in cosmetics for hair.

### Technical Solution

As a result of diligent studies in order to achieve the aforementioned objectives, the inventors of the present invention have completed the present invention. The objective of the present invention can be achieved by a cosmetic for hair comprising (A) a sugar alcohol-modified silicone represented by the following general formula (1):

R¹ₐR²_{b}L¹_{c}Q_{d}SiO_{(4-a-b-c-d)/2} (1)

wherein
R¹ represents a monovalent organic group, with the proviso that R², L and Q are excluded therefrom;
R² represents a substituted or non-substituted, and linear or branched monovalent hydrocarbon group having 9 to 30 carbon atoms, or a linear organosiloxane group represented by the following general formula (2-1):

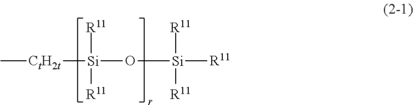

wherein R¹¹ is a substituted or non-substituted monovalent hydrocarbon group having 1 to 30 carbon atoms, a hydroxyl group or a hydrogen atom, and at least one R¹¹ is the aforementioned monovalent hydrocarbon group; t is a number ranging from 2 to 10; and r is a number ranging from 1 to 500,
or represented by the following general formula (2-2):

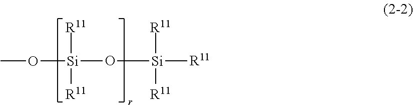

wherein R¹¹ and r are the same as defined above;
L¹ represents a silylalkyl group having a siloxane dendron structure, in the case of i=1, represented by the following general formula (3):

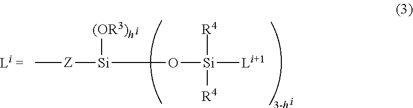

wherein
R³ represents a substituted or non-substituted, and linear or branched monovalent hydrocarbon group having 1 to 30 carbon atoms;
R⁴ independently represents an alkyl group having 1 to 6 carbon atoms or a phenyl group;
Z represents a divalent organic group;
i specifies the number of generations of the aforementioned silylalkyl group, represented by Lⁱ, in the case in which the number of generations of the aforementioned silylalkyl group, which is the number of repetitions of the aforementioned silylalkyl group, is k, i is an integer ranging from 1 to k, and the number of generations k is an integer ranging from 1 to 10;
Lⁱ⁺¹ is the aforementioned silylalkyl group in the case of i <k, and Lⁱ⁺¹ is R⁴ in the case of i=k; and hⁱ is a number ranging from 0 to 3;
Q represents an organic group containing a sugar alcohol group; and
each of a, b, c and d is independently a number having the following range: 1.0≦a≦2.5, 0≦b≦1.5, 0≦c≦1.5, and 0.0001≦d≦1.5.

In the aforementioned general formula (1), the monovalent organic group, which is R¹, preferably represents a substituted or non-substituted, and linear or branched monovalent hydrocarbon group having 1 to 8 carbon atoms, a polyoxyalkylene group represented by the following formula: —R⁵O(AO)ₙR⁶ wherein AO represents an oxyalkylene group having 2 to 4 carbon atoms; R⁵ represents a substituted or non-substituted, and linear or branched divalent hydrocarbon group having 3 to 5 carbon atoms; R⁶ represents a hydrogen atom, a substituted or non-substituted, and linear or branched monovalent hydrocarbon group having 1 to 24 carbon atoms, or a substituted or non-substituted, and linear or branched acyl group having 2 to 24 carbon atoms; and n=1 to 100, an alkoxy group, a hydroxyl group or a hydrogen atom, with the proviso that all R¹s do not represent a hydroxyl group, a hydrogen atom, the aforementioned alkoxy group or the aforementioned polyoxyalkylene group.

In the aforementioned general formula (1), Q is preferably an organic group containing a sugar alcohol group represented by the following general formula (4-1):

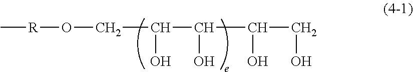

wherein R represents a divalent organic group; and e is 1 or 2,
or represented by the following general formula (4-2):

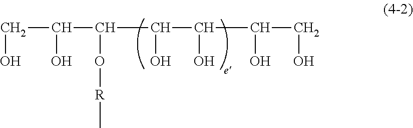

wherein R is the same as defined above; and e′ is 0 or 1.

In the aforementioned general formula (4-1) or (4-2), the divalent organic group, which is R, is preferably a substituted or non-substituted, and linear or branched divalent hydrocarbon group having 3 to 5 carbon atoms.

In the aforementioned general formula (1), the silylalkyl group having a siloxane dendron structure, represented by L, is preferably a functional group represented by the following general formula (3-1):

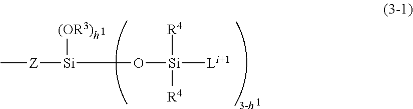

or represented by the following general formula (3-2):

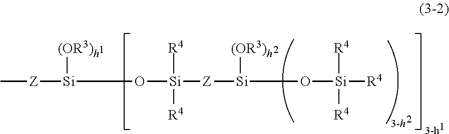

wherein R³, R⁴ and Z are the same as defined above; and each of h¹ and h² is independently a number ranging from 0 to 3.

The aforementioned (A) sugar alcohol-modified silicone of the present invention is preferably represented by the following structural formula (1-1):

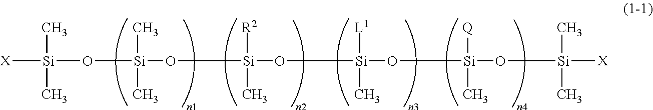

wherein
R², L¹ and Q are the same as defined above;
X is a group selected from the group consisting of a methyl group, R², L¹ and Q;
each of n1, n2, n3 and n4 is independently a number ranging from 0 to 2,000, and n1+n2+n3+n4 is a number ranging from 1 to 2,000, with the proviso that in the case of n4=0, at least one X is Q.

The aforementioned (A) sugar alcohol-modified silicone is more preferably represented by the following structural formula (1-1-1):

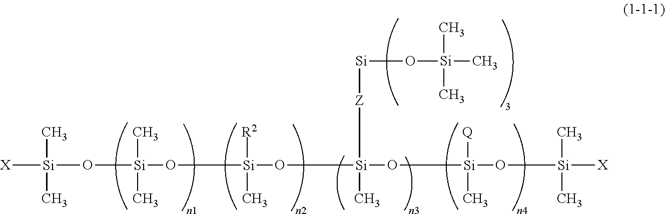

wherein R², Q, X, Z, n1, n2, n3 and n4 are the same as defined above,
or represented by the following structural formula (1-1-2):

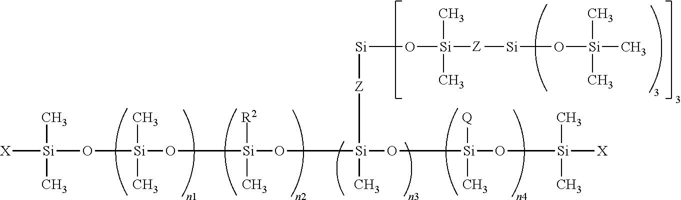

wherein R², Q, X, Z, n1, n2, n3, and n4 are the same as defined above.

In the aforementioned structural formula (1-1-1) or structural formula (1-1-2), Z is independently and preferably a group selected from divalent organic groups represented by the following general formulae:

—R⁷—

—R⁷—CO—

—R⁷—COO—R⁸—

—CO—R⁷—

—R⁷—COO—R⁸—

—R⁷—CONH—R⁸—

—R⁷—R⁸—

wherein
each R⁷ independently represents a substituted or non-substituted, and linear or branched, alkylene or alkenylene group having 2 to 22 carbon atoms or an arylene group having 6 to 22 carbon atoms;
R⁸ is a group selected from the group consisting of the following groups:

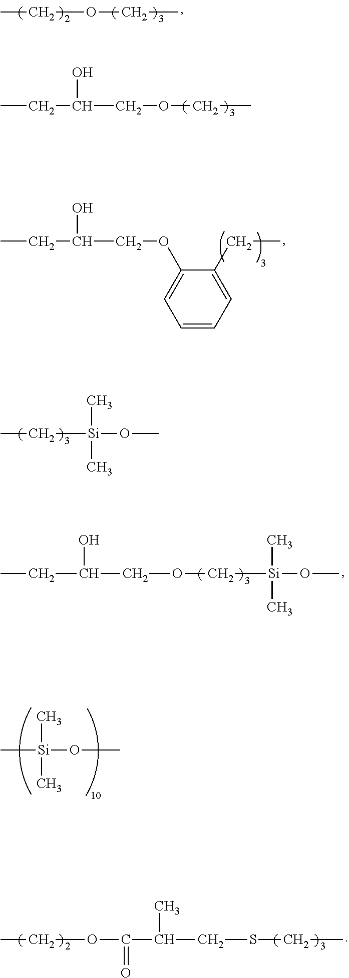

The cosmetic for hair of the present invention preferably further comprises (B) an oil agent.

The cosmetic for hair of the present invention preferably further comprises (C) a surfactant.

The cosmetic for hair of the present invention preferably further comprises (D) a water-soluble polymer.

The cosmetic for hair of the present invention can be in the form of a cosmetic for cleansing hair, a cosmetic for conditioning hair, a cosmetic for styling hair, or a cosmetic for dyeing hair.

The cosmetic for cleansing hair of the present invention preferably further comprises (C1) an anionic surfactant and (D1) a cationic water-soluble polymer.

The cosmetic for conditioning hair of the present invention preferably further comprises (B2-1) a higher alcohol and (C2) a cationic surfactant.

The cosmetic for styling hair of the present invention is preferably in the form of a liquid, a cream, a solid, a paste, a gel, a mousse, or a spray.

The cosmetic for dyeing hair of the present invention preferably further comprises (K) an oxidation hair-dyeing agent and/or (L) a direct dye.

### Advantageous Effects of Invention

The cosmetics for hair of the present invention exhibit a superior sensation during use such as a non-sticky sensation, smoothness or the like, superior effects of styling and/or conditioning hair after use such as set retention ability, a feeling on touch of hair after drying, and/or superior cleansing properties such as foaming properties, foaming quality and the like.

In particular, cosmetics for hair of the present invention are smooth, can be easily applied, and can exhibit a superior sensation during use. In addition, a smooth feeling on touch and/or combability with fingers and/or combability with a comb can be provided without an uncomfortable sticky sensation and without a frictional sensation, both during wetting and during drying. A refreshing natural feeling on touch can be provided to hair. In addition, flexibility, a styling sensation, and setting ability can be provided to hair, and the effects thereof can be maintained. Furthermore, good foaming properties and/or a good feeling on touch can also be exhibited. Therefore, the cosmetics for hair of the present invention may be preferably used as a cosmetic for cleansing hair, a cosmetic for conditioning hair, a cosmetic for styling hair, or a cosmetic for dyeing hair.

In addition, the aforementioned sugar alcohol-modified silicone exhibits superior miscibility with each of components contained in the cosmetic for hair. For this reason, the cosmetic for hair of the present invention can exhibit superior stability and in particular, can exhibit superior emulsification stability.

### BEST MODES FOR CARRYING OUT THE INVENTION

The cosmetic for hair of the present invention comprises a sugar alcohol-modified silicone represented by the following general formula (1):

R¹ₐR²_{b}L¹_{c}O_{d}SiO_{(4-a-b-c-d)/2} (1)

wherein
R¹ represents a monovalent organic group, with the proviso that R², L¹ and Q are excluded therefrom;
R² represents a substituted or non-substituted, and linear or branched monovalent hydrocarbon group having 9 to 30 carbon atoms, or a linear organosiloxane group represented by the following general formula (2-1):

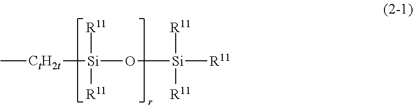

wherein R¹¹ is a substituted or non-substituted monovalent hydrocarbon group having 1 to 30 carbon atoms, a hydroxyl group or a hydrogen atom, and at least one R¹¹ is the aforementioned monovalent hydrocarbon group; t is a number ranging from 2 to 10; and r is a number ranging from 1 to 500,
or represented by the following general formula (2-2):

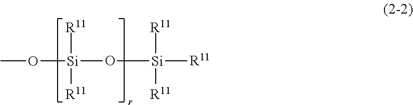

wherein R¹¹ and r are the same as defined above;
L¹ represents a silylalkyl group having a siloxane dendron structure, in the case of i=1, represented by the following general formula (3):

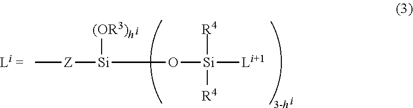

wherein
R³ represents a substituted or non-substituted, and linear or branched monovalent hydrocarbon group having 1 to 30 carbon atoms;
R⁴ independently represents an alkyl group having 1 to 6 carbon atoms or a phenyl group;
Z represents a divalent organic group;
i specifies the number of generations of the aforementioned silylalkyl group, represented by Lⁱ, in the case in which the number of generations of the aforementioned silylalkyl group, which is the number of repetitions of the aforementioned silylalkyl group, is k, i is an integer ranging from 1 to k, and the number of generations k is an integer ranging from 1 to 10;
Lⁱ⁺¹ is the aforementioned silylalkyl group in the case of i<k, and Lⁱ⁺¹ is R⁴ in the case of i=k; and hⁱ is a number ranging from 0 to 3;
Q represents an organic group containing a sugar alcohol group; and
each of a, b, c and d is independently a number having the following range: 1.0≦a≦2.5, 0≦b≦1.5, 0≦c≦1.5, and 0.0001≦d≦1.5.

The monovalent organic group, which is R¹ of the aforementioned general formula (1), is not particularly restricted as long as the monovalent organic group is not a functional group corresponding to L¹, R² or Q. The monovalent organic group preferably represents a substituted or non-substituted, and linear or branched monovalent hydrocarbon group having 1 to 8 carbon atoms, a polyoxyalkylene group represented by the following formula: —R⁵O(AO)ₙR⁶ (wherein AO represents an oxyalkylene group having 2 to 4 carbon atoms; R⁵ represents a substituted or non-substituted, and linear or branched divalent hydrocarbon group having 3 to 5 carbon atoms; R⁶ represents a hydrogen atom, a substituted or non-substituted, and linear or branched monovalent hydrocarbon group having 1 to 24 carbon atoms, or a substituted or non-substituted, and linear or branched acyl group having 2 to 24 carbon atoms; and n=1 to 100), an alkoxy group, a (meth)acryl group, an amide group, a carbinol group or a phenol group, with the proviso that all R¹s do not represent a hydroxyl group, a hydrogen atom, the aforementioned alkoxy group or the aforementioned polyoxyalkylene group.

As examples of monovalent hydrocarbon groups having 1 to 8 carbon atoms, mention may be made of, for example, alkyl groups such as a methyl group, an ethyl group, a propyl group, a butyl group, a pentyl group, a hexyl group, a heptyl group, an octyl group and the like; cycloalkyl groups such as a cyclopentyl group, a cyclohexyl group and the like; alkenyl groups such as a vinyl group, an allyl group, a butenyl group and the like; aryl groups such as a phenyl group, a tolyl group and the like; aralkyl groups such as a benzyl group and the like; substituted groups thereof in which the hydrogen atoms binding to the carbon atoms of the aforementioned groups are at least partially substituted with a halogen atom such as a fluorine atom or the like, or an organic group containing an epoxy group, a glycidyl group, an acyl group, a carboxyl group, an amino group, a methacryl group, a mercapto group or the like, with the proviso that the total number of the carbon atoms ranges from 1 to 8 carbon atoms. The monovalent hydrocarbon group is preferably a group other than an alkenyl group, and a methyl group, an ethyl group or a phenyl group is, in particular, preferred. In addition, as examples of alkoxy groups, mention may be made of lower alkoxy groups such as a methoxy group, an ethoxy group, an isopropoxy group, a butoxy group and the like; higher alkoxy groups such as a laurylalkoxy group, a myristylalkoxy group, a palmitylalkoxy group, an oleylalkoxy group, a stearylalkoxy group, a behenylalkoxy group and the like.

In particular, R¹ is preferably a monovalent hydrocarbon group or a monovalent fluorinated hydrocarbon group, having 1 to 8 carbon atoms and having no aliphatic unsaturated bond. As examples of the monovalent hydrocarbon group having no aliphatic unsaturated bond belonging to R¹, mention may be made of alkyl groups such as a methyl group, an ethyl group, a propyl group, a butyl group, a pentyl group, a hexyl group and the like; aryl groups such as a phenyl group, a tolyl group, a xylyl group and the like; and aralkyl groups such as a benzyl group and the like. As examples of monovalent fluorinated hydrocarbon groups, mention may be made of perfluoroalkyl groups such as a trifluoropropyl group, a pentafluoroethyl group and the like. From an industrial point of view, R¹ is preferably a methyl group, an ethyl group, or a phenyl group, and in particular, 90% by mol to 100% by mol of all R¹s is preferably a group selected from the group consisting of a methyl group, an ethyl group and a phenyl group.

In the aforementioned sugar alcohol-modified silicone, in order to provide a further functional property, a modified group other than the hydrophilic group (-Q), and in particular, a short-chain or medium-chain hydrocarbon-based group may be introduced as R¹, and the organopolysiloxane can be designed. Namely, in the case of R¹ being a substituted monovalent hydrocarbon group, a substituent can be appropriately selected in accordance with a property and a usage to be provided. For example, in the case of using the sugar alcohol-modified silicone as a raw material of a cosmetic, for the purpose of improving a sensation during use, a feeling on touch, and durability, an amino group, an amide group, an aminoethyl aminopropyl group, a carboxyl group or the like can be introduced as a substituent of the monovalent hydrocarbon group.

R² of the aforementioned general formula (1) is a substituted or non-substituted, and linear or branched monovalent hydrocarbon group having 9 to 30 carbon atoms as a long-chain hydrocarbon group or a linear organosiloxane group represented by the aforementioned general formula (2-1) or (2-2). By introducing into the main chain and/or the side chain of the polysiloxane, an emulsifying property and a dispersing property with respect to an oil agent, a powder and the like to be blended into a hair cosmetic can be further improved. In addition, a sensation during use of a hair cosmetic can also be further improved. In addition, the aforementioned monovalent long-chain hydrocarbon group or linear organopolysiloxane group is a hydrophobic functional group, and for this reason, miscibility and/or blending stability with respect to an organic oil having an increased amount of an alkyl group can be further improved. All R²s may be the aforementioned monovalent long-chain hydrocarbon group or linear organopolysiloxane group, and may also be both of the aforementioned functional groups. In the aforementioned sugar alcohol-modified silicone, in particular, a part or all of the R²s is/are preferably a monovalent long-chain hydrocarbon group. By possessing the aforementioned monovalent long-chain hydrocarbon group in a molecule, the sugar alcohol-modified silicone of the present invention can exhibit superior miscibility with respect to not only a silicone oil, but also a non-silicone oil having an increased amount of an alkyl group. For example, an emulsion or dispersion formed from a non-silicone oil, which exhibits superior thermal stability and superior stability over time, can be obtained.

The substituted or non-substituted, and linear or branched monovalent hydrocarbon groups having 9 to 30 carbon atoms, binding to a silicon atom, represented by R² of the aforementioned general formula (1) may be the same or different, and the structure thereof is selected from a linear structure, a branched structure, and a partially branched structure. In the present invention, in particular, a non-substituted and linear monovalent hydrocarbon group is preferably used. As examples of non-substituted monovalent hydrocarbon groups, mention may be made of, for example, an alkyl group, an aryl group, or an aralkyl group, having 9 to carbon atoms, and preferably having 10 to 25 carbon atoms. On the other hand, as examples of substituted monovalent hydrocarbon groups, mention may be made of, for example, a perfluoroalkyl group, an aminoalkyl group, an amidoalkyl group, and a carbinol group, having 9 to 30 carbon atoms, and preferably having 10 to 25 carbon atoms. In addition, a part of the carbon atoms of the aforementioned monovalent hydrocarbon group may be substituted with an alkoxy group, and as examples thereof, mention may be made of, a methoxy group, an ethoxy group, and a propoxy group. The aforementioned monovalent hydrocarbon group is, in particular, preferably an alkyl group having 9 to 30 carbon atoms, and examples thereof include a group represented by the following general formula: —(CH₂)ᵥ—CH₃ wherein v is a number ranging from 8 to 30. An alkyl group having 10 to 25 carbon atoms is, in particular, preferred.

The linear organosiloxane group represented by the aforementioned general formula (2-1) or (2-2) is different from the silylalkyl group having a siloxane dendron structure, and has a linear polysiloxane chain structure. In the aforementioned general formula (2-1) or (2-2), each R¹¹ is independently a substituted or non-substituted monovalent hydrocarbon group having 1 to 30 carbon atoms, a hydroxyl group or a hydrogen atom. The substituted or non-substituted monovalent hydrocarbon group having 1 to 30 carbon atoms is preferably an alkyl group having 1 to 30 carbon atoms, an aryl group having 6 to 30 carbon atoms, an aralkyl group having 6 to 30 carbon atoms, or a cycloalkyl group having 6 to 30 carbon atoms. As examples thereof, mention may be made of alkyl groups such as a methyl group, an ethyl group, a propyl group, a butyl group, a pentyl group, a hexyl group, a heptyl group, an octyl group, a decyl group and the like; cycloalkyl groups such as a cyclopentyl group, a cyclohexyl group and the like; and aryl groups such as a phenyl group, a tolyl group and the like. The hydrogen atoms binding to the carbon atoms of the aforementioned groups may be at least partially substituted with a halogen atom such as a fluorine atom or the like, or an organic group containing an epoxy group, an acyl group, a carboxyl group, an amino group, a methacryl group, a mercapto group and the like. As examples of particularly preferable groups as R¹¹, mention may be made of a methyl group, a phenyl group or a hydroxyl group. A mode in which a part of R¹¹ is a methyl group, and another part thereof is a long-chain alkyl group having 8 to 30 carbon atoms is also preferred.

In the aforementioned general formula (2-1) or (2-2), each t is a number ranging from 2 to 10, r is a number ranging from 1 to 500, and r is preferably a number ranging from 2 to 500. The aforementioned linear organosiloxane group is hydrophobic, and in view of miscibility with various oil agents, r is preferably a number ranging from 1 to 100, and more preferably a number ranging from 2 to 30.

The aforementioned silylalkyl group having a siloxane dendron structure includes a structure in which carbosiloxane units are spread in the form of a dendrimer, and is a functional group exhibiting increased water-repellency. Superior balance with a hydrophilic group is exhibited. At the time of using a cosmetic for hair blending the aforementioned sugar alcohol-modified silicone, an uncomfortable sticky sensation can be controlled, and a refreshing and natural feeling on touch can be provided. In addition, the silylalkyl group having the aforementioned siloxane dendron structure is chemically stable, and for this reason, the aforementioned silylalkyl group is a functional group which is capable of imparting an advantageous property that widely-ranged cosmetic ingredients can be used in combination therewith.

As examples of substituted or non-substituted, and linear or branched monovalent hydrocarbon groups having 1 to carbon atoms, represented by R³ of the aforementioned general formula (3), mention may be made of, for example, alkyl groups such as a methyl group, an ethyl group, a propyl group, a butyl group, a pentyl group, a hexyl group, a heptyl group, an octyl group and the like; cycloalkyl groups such as a cyclopentyl group, a cyclohexyl group and the like; alkenyl groups such as a vinyl group, an allyl group, a butenyl group and the like; aryl groups such as a phenyl group, a tolyl group and the like; aralkyl groups such as a benzyl group and the like; substituted groups thereof in which the hydrogen atoms binding to the carbon atoms of the aforementioned groups are at least partially substituted with a halogen atom such as a fluorine atom or the like, or an organic group containing an epoxy group, a glycidyl group, an acyl group, a carboxyl group, an amino group, a methacryl group, a mercapto group or the like, with the proviso that the total number of the carbon atoms ranges from 1 to 30 carbon atoms.

Among alkyl groups having 1 to 6 carbon atoms and a phenyl group, represented by R⁴ in the aforementioned general formula (3), as examples of alkyl groups having 1 to 6 carbon atoms, mention may be made of linear, branched or cyclic alkyl groups such as a methyl group, an ethyl group, a n-propyl group, an i-propyl group, a n-butyl group, an i-butyl group, a s-butyl group, a pentyl group, a neopentyl group, a cyclopentyl group, a hexyl group and the like.

In the aforementioned general formula (3), in the case of i=k, R⁴ is preferably a methyl group or a phenyl group. In particular, in the case of i=k, R⁴ is preferably a methyl group.

The aforementioned number of generations k is preferably an integer ranging from 1 to 3, and more preferably 1 or 2 from an industrial viewpoint. In each number of generations, the group represented by L¹ is represented as follows, wherein R³, R⁴ and Z are the same groups as described above.

In the case of the number of generations k=1, L¹ is represented by the following general formula (3-1):

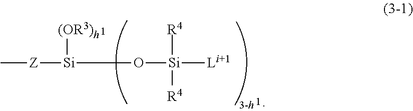

In the case of the number of generations k=2, L¹ is represented by the following general formula (3-2):

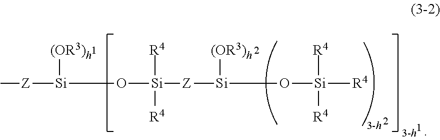

In the case of the number of generations k=3, L¹ is represented by the following general formula (3-3):

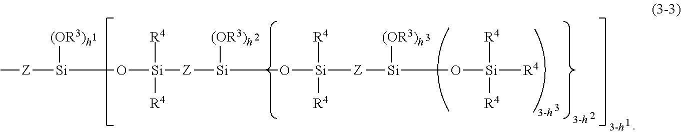

In the structures represented by the aforementioned general formulae (3-1) to (3-3) in the case of the number of generations ranging from 1 to 3, each of h¹, h² and h³ is independently a number ranging from 0 to 3. The aforementioned hⁱ is preferably a number particularly ranging from 0 to 1, and hⁱ is, in particular, preferably 0.

In the aforementioned general formulae (3) and (3-1) to (3-3), each Z is independently a divalent organic group. In particular, as examples thereof, mention may be made of a divalent organic group formed by addition-reacting a silicon-binding hydrogen atom and a functional group having an unsaturated hydrocarbon group such as an alkenyl group, an acryloxy group, a methacryloxy group or the like at the terminal. In accordance with the method of introducing a silylalkyl group having a siloxane dendron structure, the functional groups can be appropriately selected and are not restricted to the aforementioned functional groups. Preferably, each Z is independently a group selected from divalent organic groups represented by the following general formulae:

—R⁷—

—R⁷—CO—

—R⁷—COO—R⁸—

—CO—R⁷—

—R⁷—COO—R⁸—

—R⁷—CONH—R⁸—

—R⁷—R⁸—

In particular, Z in L¹ is preferably a divalent organic group represented by the following general formula: —R⁷—, introduced by a reaction between a silicon-binding hydrogen atom and an alkenyl group. In the same manner, Z is preferably a divalent organic group represented by the following general formula: —R⁷—COO—R⁸—, introduced by a reaction between a silicon-binding hydrogen atom and an unsaturated carboxylic ester group. On the other hand, in the silylalkyl group represented by Lⁱ, in which the number of generations k is 2 or more, and Lⁱ is L² to L^{k}, Z is preferably an alkylene group having 2 to 10 carbon atoms, in particular, preferably a group selected from an ethylene group, a propylene group, a methylethylene group and a hexylene group, and most preferably an ethylene group.

In the aforementioned general formulae, each R⁷ independently represents a substituted or non-substituted, and linear or branched alkylene or alkenylene group having 2 to 22 carbon atoms, or an arylene group having 6 to 22 carbon atoms. More particularly, as examples of R⁷, mention may be made of linear alkylene groups such as an ethylene group, a propylene group, a butylene group, a hexylene group and the like; and branched alkylene groups such as a methylmethylene group, a methylethylene group, a 1-methylpentylene group, a 1,4-dimethylbutylene group and the like. R⁷ is preferably a group selected from an ethylene group, a propylene group, a methylethylene group and a hexylene group.

In the aforementioned general formulae, R⁸ is a group selected from divalent organic groups represented by the following formulae:

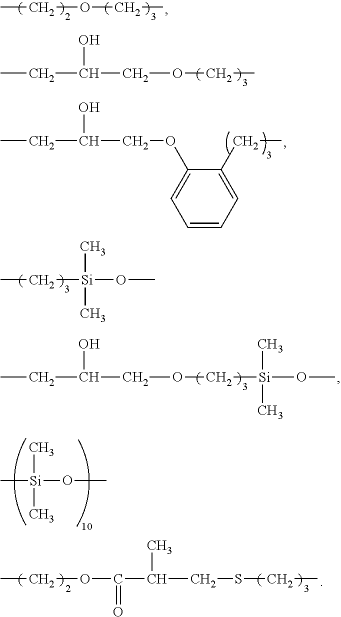

In the aforementioned general formula (1), Q is a sugar alcohol-containing organic group, and constitutes a hydrophilic moiety of the sugar alcohol-modified silicone of the present invention. Q is not particularly restricted in the structure as long as the structure has a sugar alcohol moiety. In Q, a sugar alcohol residue is preferably bound to a silicon atom via a divalent organic group.

Therefore, Q is preferably represented by the following general formula (4-1):

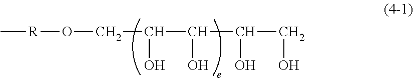

wherein
R represents a divalent organic group; and
e is 1 or 2,
or represented by the following general formula (4-2):

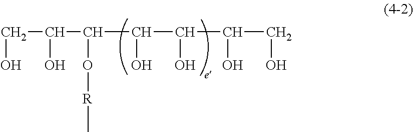

wherein
R is the same as defined above; and
e′ is 0 or 1.

The sugar alcohol-modified silicone according to the present invention is characterized in that among the sugar alcohol-containing organic groups represented by the aforementioned general formula (4-1) or (4-2), at least one type of the groups binds to a silicon atom. In addition, the sugar alcohol-modified silicone may be an organopolysiloxane in which two or more types of sugar alcohol-containing organic groups selected from the aforementioned sugar alcohol-containing organic groups are possessed in an identical molecule. In the same manner, a mixture of the organopolysiloxanes having different sugar alcohol-containing organic groups may be used.

The divalent organic group represented by R of the aforementioned general formula (4-1) or (4-2) is not particularly restricted, and as an example thereof, mention may be made of a substituted or non-substituted, and linear or branched divalent hydrocarbon group having 1 to 30 carbon atoms. A substituted or non-substituted, and linear or branched divalent hydrocarbon group having 3 to 5 carbon atoms is preferred. As examples of the substituted or non-substituted, and linear or branched divalent hydrocarbon group having 1 to 30 carbon atoms, mention may be made of, for example, linear or branched alkylene groups having 1 to carbon atoms such as a methylene group, a dimethylene group, a trimethylene group, a tetramethylene group, a pentamethylene group, a hexamethylene group, a heptamethylene group, an octamethylene group and the like; alkenylene groups having 2 to 30 carbon atoms such as a vinylene, an allylene group, a butenylene group, a hexenylene group, an octenylene group and the like; arylene groups having 6 to 30 carbon atoms such as a phenylene group, a diphenylene group and the like; alkylenearylene groups having 7 to 30 carbon atoms such as a dimethylenephenylene group and the like; and substituted groups thereof in which hydrogen atoms binding to carbon atoms of the aforementioned groups are at least partially substituted by a halogen atom such as a fluorine atom or the like, or an organic group containing a carbinol group, an epoxy group, a glycidyl group, an acyl group, a carboxyl group, an amino group, a methacryl group, a mercapto group, an amide group, an oxyalkylene group or the like. The divalent hydrocarbon group is preferably an alkylene group having 1 to 30 carbon atoms, more preferably an alkylene group having 1 to 6 carbon atoms, and further preferably an alkylene group having 3 to 5 carbon atoms.

As the sugar alcohol-containing organic group, the case in which R is a propylene group and e=1 in the aforementioned general formula (4-1) is, in particular, preferred. In the same manner as described above, as the sugar alcohol-containing organic group, the case in which R is a propylene group and e′=0 in the aforementioned general formula (4-2) is, in particular, preferred. The sugar alcohol-containing organic group in this case is a xylitol residue (hereinafter, merely referred to as “xylitol residue” or “xylitol-modified group”) represented by the following structural formula: —C₃H₆—OCH₂[CH(OH)]₃CH₂OH or the following structural formula: —C₃H₆—OCH{CH(OH)CH₂OH}₂, respectively in the case of the aforementioned general formula (4-1) or (4-2)

The binding site of the sugar alcohol-containing organic group may be any one of the side chains or the terminals of the polysiloxane which is the main chain. A structure in which two or more sugar alcohol-containing organic groups are present in one molecule of the sugar alcohol-modified silicone may be used. In addition, the aforementioned two or more sugar alcohol-containing organic groups may be the same or different sugar alcohol-containing organic groups. A structure in which the aforementioned two or more sugar alcohol-containing organic groups bind to only the side chains, only the terminals, or both the side chain and the terminal of the polysiloxane which is the main chain may be used.

A sugar alcohol-modified silicone possessing a sugar alcohol group-containing organic group (-Q), represented by the aforementioned general formula (1) is preferably a sugar alcohol-modified silicone possessing a linear polysiloxane structure represented by the following structural formula (1-1):

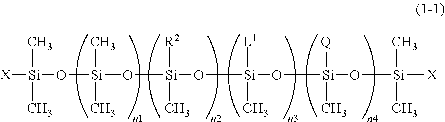

wherein
R², L¹ and Q are the same as defined above;
X is a group selected from the group consisting of a methyl group, R², L¹ and Q;
each of n1, n2, n3 and n4 is independently a number ranging from 0 to 2,000, and n1+n2+n3+n4 is a number ranging from 1 to 2,000, with the proviso that in the case of n4=0, at least one X is Q.

In the aforementioned formula (1-1), (n1+n2+n3+n4) is preferably a number ranging from 10 to 2,000, preferably a number ranging from 25 to 1,500, and in particular, preferably a number ranging from 50 to 1,000. n1 is preferably a number ranging from 10 to 2,000, more preferably a number ranging from 25 to 1,500, and further preferably a number ranging from 50 to 1,000. n2 is preferably a number ranging from 0 to 250, and more preferably a number ranging from 0 to 150.

In the case of R² being the aforementioned long-chain alkyl group, in particular, n2>1 is preferred in view of surface activity and miscibility with oil agents other than silicones. n3 is preferably a number ranging from 0 to 250, and in particular, it is preferred that n3>1 and one or more silylalkyl groups (-L¹) having a siloxane dendron structure at the side chain part be possessed. n4 is a number ranging from 0 to 100, and preferably a number ranging from 0 to 50, with the proviso that in the case of n4=0, at least one X must be Q. In view of capability of imparting a smooth feeling on touch and a film thickness sensation to hair and durability of the aforementioned effects for a long time, an increased molecular weight is effective. For example, by use of a sugar alcohol-modified silicone with an increased molecular weight, reduction of the effects during brushing or treating with a dryer does not occur much, and effects of preventing a frictional sensation or a sticky sensation after drying can be exhibited.

In the aforementioned structural formula (1-1), each Q is independently a sugar alcohol-containing organic group represented by the aforementioned general formula (4-1) or general formula (4-2). In the aforementioned sugar alcohol-modified silicone, all Qs may be sugar alcohol-containing organic groups represented by the aforementioned general formula (4-1) or general formula (4-2), or alternatively, a part of Q in one molecule may be a sugar alcohol-containing organic group represented by the aforementioned general formula (4-1), and the remaining Q may be a sugar alcohol-containing organic group represented by the aforementioned general formula (4-2).

In addition, the sugar alcohol-modified silicone may be one type of the aforementioned sugar alcohol-modified silicone represented by the aforementioned general formula (1) or a mixture of two or more types thereof.

In particular, in the aforementioned sugar alcohol-modified silicone, represented by the aforementioned general formula (1), Q is preferably a xylitol residue.

As described above, the xylitol residue is a group represented by the structural formula: —C₃H₆—OCH₂[CH(OH)]₃CH₂OH or the structural formula: —C₃H₆—OCH{CH(OH)CH₂OH}₂. In the sugar alcohol-modified silicone according to the present invention, the aforementioned xylitol residues may be one type or two types. Therefore, in the aforementioned general formula (1), all Qs may consist of only the xylitol residue represented by the structural formula: —C₃H₆—OCH₂[CH(OH)]₃CH₂OH or the structural formula: —C₃H₆—OCH{CH(OH)CH₂OH}₂, or alternatively, Qs may consist of two types of xylitol residues represented by the structural formula: —C₃H₆—OCH₂[CH(OH)]₃CH₂OH and represented by the structural formula: —C₃H₆—OCH{CH(OH)CH₂OH}₂. In the latter case, the composition ratio (weight ratio) preferably ranges from 5:5 to 10:0, and in particular, preferably ranges from 8:2 to 10:0. The case of 10:0 means that Q substantially consists of only a xylitol residue represented by the structural formula: —C₃H₆—OCH₂[CH(OH)]₃CH₂OH.

In addition, in the case in which the aforementioned sugar alcohol-modified silicone is a mixture of two or more types of sugar alcohol-modified silicones, the aforementioned mixture can comprise at least two types of sugar alcohol-modified silicones selected from the group consisting of a sugar alcohol-modified silicone in which Q in the aforementioned general formula (1) consists of only a xylitol residue represented by the structural formula: —C₃H₆—OCH₂[CH(OH)]₃CH₂OH, a sugar alcohol-modified silicone in which Q in the aforementioned general formula (1) consists of only a xylitol residue represented by the structural formula: —C₃H₆—OCH{CH(OH)CH₂OH}₂, and a sugar alcohol-modified silicone in which Q in the aforementioned general formula (1) consists of two types of xylitol residues represented by the structural formula: —C₃H₆—OCH₂[CH(OH)]₃CH₂OH and the structural formula: —C₃H₆—OCH{CH(OH)CH₂OH}₂ in a constitutional ratio (weight ratio) preferably ranging from 5:5 to 10:0 and in particular, preferably ranging from 8:2 to 10:0. In addition, the sugar alcohol-modified silicone may be a mixture of at least two types of sugar alcohol-modified silicones in which Q in the aforementioned general formula (1) consists of two types of xylitol residues represented by the structural formula: —C₃H₆—OCH₂[CH(OH)]₃CH₂OH and the structural formula: —C₃H₆—OCH{CH(OH)CH₂OH}₂ in a constitutional ratio (weight ratio) preferably ranging from 5:5 to 10:0 and in particular, preferably ranging from 8:2 to 10:0, in which the constitutional ratio is different from each other.

As the aforementioned sugar alcohol-modified silicone, a sugar alcohol-modified silicone represented by the following structural formula (1-1-1):

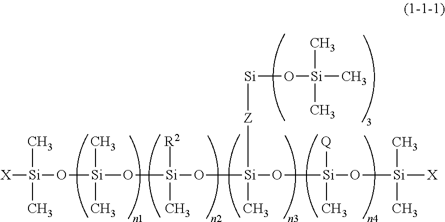

wherein
R², Q, X, Z, n1, n2, n3 and n4 are the same as defined above, or represented by the following structural formula (1-1-2):

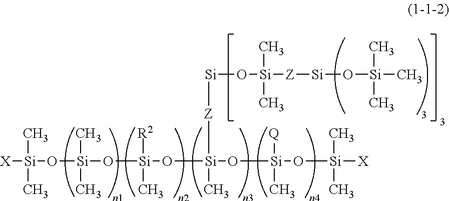

wherein
R², Q, X, Z, n1, n2, n3 and n4 are the same as defined above, is preferred.

A modification index of an organopolysiloxane with a sugar alcohol-containing organic group preferably ranges from 0.001 to 20% by mol, more preferably ranges from 0.005 to 10% by mol, and further preferably ranges from 0.01 to 5% by mol, among all functional groups binding to the polysiloxane which is the main chain. In the sugar alcohol-modified silicone represented by the aforementioned structural formula (1-1), the modification index with a sugar alcohol-containing organic group is indicated by the following equation:

Modification index(% by mol)=100×(the number of sugar alcohol-containing organic groups binding to a silicon atom in one molecule)/{6+2×(n1+n2+n3+n4)}.

For example, in the case of a sugar alcohol-modified silicone formed from a trisiloxane possessing one sugar alcohol-containing organic group, one functional group binding to a silicon atom among eight functional groups binding to silicon atoms is modified with a sugar alcohol-containing organic group. For this reason, the modification index with a sugar alcohol-containing organic group is 12.5% by mol.

The aforementioned sugar alcohol-modified silicone can be obtained by reacting (a) an organopolysiloxane having hydrogen atoms binding to silicon atoms, (b) an organic compound having one reactive unsaturated group in one molecule, (c) a sugar alcohol-functional organic compound having one reactive unsaturated group in one molecule, (d) a siloxane dendron compound having one reactive unsaturated group in one molecule, and/or (e) a long-chain hydrocarbon compound having one reactive unsaturated group in one molecule or a linear organopolysiloxane having one reactive unsaturated group in one molecule, in the presence of a catalyst for a hydrosilylation reaction. As preferable examples of the aforementioned reactive unsaturated group, mention may be made of an alkenyl group or an unsaturated fatty acid ester group, which is an unsaturated functional group having a carbon-carbon double bond. The aforementioned —R¹ is introduced by the aforementioned component (b), the aforementioned -L¹ is introduced by the aforementioned component (d), and the aforementioned —R² is introduced by the aforementioned component (e).

The aforementioned (c) sugar alcohol-functional organic compound having one reactive unsaturated group in one molecule can be replaced with a ketal derivative of a sugar alcohol compound which has a reactive unsaturated group in a molecule and in which a hydroxyl group is protected, and the ketal derivative can be used as a raw material. In this case, the ketal derivative is subjected to an addition reaction to an organopolysiloxane having a silicon-hydrogen bond, followed by subjecting to an acid hydrolyzing treatment to deprotect the hydroxyl group. Thereby, a sugar alcohol-modified siloxane according to the present invention can be produced.

The aforementioned sugar alcohol-modified siloxane can be obtained, for example, in the following manner.

The aforementioned sugar alcohol-modified siloxane can be obtained by addition-reacting an organopolysiloxane having silicon-hydrogen bonds with an unsaturated organic compound having a carbon-carbon double bond at one terminal of the molecular chain, and an unsaturated ether compound of a sugar alcohol having a carbon-carbon double bond in the molecule. In addition, a siloxane dendron compound having a carbon-carbon double bond at one terminal of the molecular chain, and/or an unsaturated long-chain hydrocarbon compound having a carbon-carbon double bond at one terminal of the molecular chain or a linear organopolysiloxane having a carbon-carbon double bond at one terminal of the molecular chain can be further subjected to an addition reaction.

In the case described above, the aforementioned sugar alcohol-modified siloxane can be obtained as a hydrosilylation reaction product between a siloxane containing SiH groups and the aforementioned unsaturated organic compound, and the aforementioned unsaturated ether compound of a sugar alcohol, as well as, optionally the aforementioned siloxane dendron compound and/or the unsaturated long chain hydrocarbon compound or the linear organopolysiloxane. Thereby, an organic group and a sugar alcohol-containing organic group, as well as, optionally a silylalkyl group having a siloxane dendron structure, and/or a long-chain hydrocarbon group or a linear organopolysiloxane group can be introduced into the polysiloxane chain of the aforementioned sugar alcohol-modified silicone.

For example, the aforementioned sugar alcohol-modified silicone can be obtained by at least reacting (a′) an organohydrogensiloxane represented by the following general formula (1′):

R¹ₐH_{b+c+d}SiO_{(4-a-b-c-d)/2} (1′)

wherein,
R¹, a, b, c and d are the same as defined above, and the aforementioned (c) sugar alcohol-functional organic compound having one reactive unsaturated group in one molecule, in the presence of a catalyst for a hydrosilylation reaction. The aforementioned (d) siloxane dendron compound having one reactive unsaturated group in one molecule, and/or the aforementioned (e) unsaturated long chain hydrocarbon compound having one reactive unsaturated group in one molecule or a linear organopolysiloxane having one reactive unsaturated group in one molecule are preferably further reacted therewith.

The aforementioned sugar alcohol-modified silicone can be preferably produced, for example, by reacting the aforementioned (c) the sugar alcohol-functional organic compound having one reactive unsaturated group in one molecule, and optionally the aforementioned (d) the siloxane dendron compound having one reactive unsaturated group in one molecule and/or the aforementioned (e) the unsaturated long chain hydrocarbon compound having one reactive unsaturated group in one molecule or a linear organopolysiloxane having one reactive unsaturated group in one molecule, as well as the aforementioned (a′) organohydrogensiloxane represented by the aforementioned general formula (1′), under the condition of (co)existing the (c) component and optionally the (d) component and/or (e) component. Alternatively, the sugar alcohol-modified silicone of can be preferably produced by additionally reacting the aforementioned (a′) organohydrogensiloxane with the other components, i.e., the components (b), (c), (d) and (e), in any sequential order.

As the aforementioned (a) organopolysiloxane having silicon atom-binding hydrogen atoms and the aforementioned (a′) organohydrogensiloxane, an organohydrogensiloxane represented by the following structural formula (1-1)′:

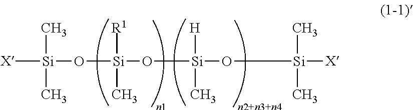

wherein
each R¹ is independently the same as defined above;
X′ is a group selected from R¹ and a hydrogen atom;
n1, n2, n3 and n4 are the same as defined above, with the proviso that in the case of n2+n3+n4=0, at least one X′ is a hydrogen atom,
is preferred.

As the aforementioned (d) siloxane dendron compound having one reactive unsaturated group in one molecule, a compound having a siloxane dendron structure having one carbon-carbon double bond at the terminal of the molecular chain, represented by the following general formula (3′):

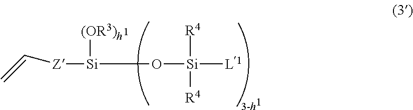

wherein
R³ and R⁴ are the same as defined above;
Z′ represents a divalent organic group;
h¹ is a number ranging from 0 to 3;
L′¹ represents R⁴ or a silylalkyl group, in the case of j=1, represented by the following general formula (3″):

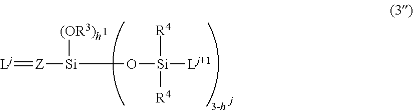

wherein R³ and R⁴ are the same as defined above;
Z represents a divalent organic group;
j specifies the number of generations of the aforementioned silylalkyl group, represented by L^{j}, in the case in which the number of generations of the aforementioned silylalkyl group, which is the number of repetitions of the aforementioned silylalkyl group, is k′, j is an integer ranging from 1 to k′, and the number of generations k′ is an integer ranging from 1 to 9;
L^{j+1} is the aforementioned silylalkyl group in the case of j<k′, and L^{j+1} is R⁴ in the case of j=k′; and h^{j} is a number ranging from 0 to 3,
is preferred.

As the aforementioned (c) sugar alcohol-functional organic compound having one reactive unsaturated group in one molecule, a mono-unsaturated ether compound of a sugar alcohol represented by the following general formula (4′-1):

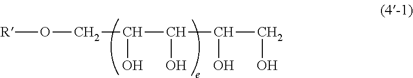

wherein
R′ represents an unsaturated organic group;
e is 1 or 2 and preferably 1,
or represented by the following general formula (4′-2):

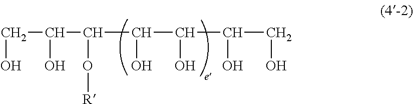

wherein
R′ represents an unsaturated organic group;
e′ is 0 or 1 and preferably 1,
is preferred.

The aforementioned unsaturated organic group is not particularly restricted as long as the organic group has an unsaturated group. A substituted or non-substituted, and linear or branched, unsaturated hydrocarbon group having 3 to 5 carbon atoms is preferred. As examples of unsaturated hydrocarbon groups having 3 to 5 carbon atoms, mention may be made of alkenyl groups such as a vinyl group, an allyl group, a butenyl group and the like. An allyl group is preferred.

As the aforementioned mono-unsaturated ether compound of a sugar alcohol, a monoallyl ether of a sugar alcohol is preferred, and xylitol monoallyl ether (hereinafter, referred to as “xylitol monoallyl ether”) represented by the following structural formula: CH₂═CH—CH₂—OCH₂[CH(OH)]₃CH₂OH or represented by the following structural formula: CH₂═CH—CH₂—OCH{CH(OH)CH₂OH}₂ is more preferred. The xylitol monoallyl ether can be synthesized in accordance with a conventional method.

As the aforementioned xylitol monoallyl ether, either one or a mixture of a compound represented by the following structural formula: CH₂═CH—CH₂—OCH₂[CH(OH)]₃CH₂OH and a compound represented by the following structural formula: CH₂═CH—CH₂—OCH{CH(OH)CH₂OH}₂ can be used without particular restriction. Preferably, either one of the xylitol monoallyl ethers represented by the following structural formula: CH₂═CH—CH₂—OCH₂[CH(OH)]₃CH₂OH and represented by the following structural formula: CH₂═CH—CH₂—OCH{CH(OH)CH₂OH}₂ is purified and used as a raw material. Alternatively, a xylitol monoallyl ether mixture containing xylitol monoallyl ethers represented by the following structural formula: CH₂═CH—CH₂—OCH₂[CH(OH)]₃CH₂OH and represented by the following structural formula: CH₂═CH—CH₂—OCH{CH(OH)CH₂OH}₂ in a weight (mass) ratio ranging from 5:5 to 10:0 is preferably used as a raw material. In the latter case, use of the xylitol monoallyl ether having a ratio ranging from 8:2 to 10:0 is more preferred. In the case of using a ratio of 10:0, the raw material is a purified product consisting substantially of the xylitol monoallyl ether represented by the following structural formula: CH₂═CH—CH₂—OCH₂[CH(OH)]₃CH₂OH.

In addition, as described above, in order to obtain the aforementioned sugar alcohol-modified silicone, a derivative of a sugar alcohol compound (a ketal compound) in which a hydroxyl group of the sugar alcohol compound corresponding to a sugar alcohol-modified group to be introduced is protected by a ketalizing agent such as 2,2-dimethoxypropane or the like in the presence of an acid catalyst, can also be used as a raw material. More particularly, the ketal derivative of the sugar alcohol having a carbon-carbon double bond in the molecule, which is obtained by purifying a reaction product between the aforementioned ketal compound and an alkenyl halide, instead of the aforementioned monounsaturated ether compound of a sugar alcohol, is subjected to an addition reaction with an organopolysiloxane having silicon-hydrogen bonds. After the addition reaction, a de-ketalization reaction can be carried out by means of an acid hydrolysis treatment to deprotect the hydroxyl group. Thereby, the aforementioned sugar alcohol-modified silicone can also be produced. Even by the aforementioned method using the aforementioned ketal derivative, after deprotection, an organopolysiloxane having a sugar alcohol-modified group can be obtained. For this reason, any one of the preparation methods may be selected in accordance with the desirable yield or the conditions such as production facilities, purification of raw materials and the like. In addition, in order to improve a quality such as purification or a desirable property of the aforementioned sugar alcohol-modified silicone, any one of the preparation methods may be selected.

As the aforementioned (e) hydrocarbon compound having one reactive unsaturated group in one molecule or the aforementioned linear organopolysiloxane having one reactive unsaturated group in one molecule, a monounsaturated organic compound represented by the following general formula:

R′—R²

wherein R¹ is the same as defined above;
R^{2′} represents a substituted or non-substituted, and linear or branched monovalent hydrocarbon group having 7 to 28 carbon atoms or a linear organosiloxane group represented by the following general formula (2-1):

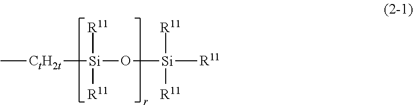

wherein R¹¹, t and r are the same as defined above, or represented by the following general formula (2-2):

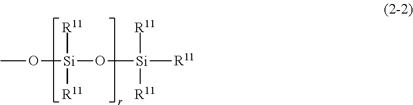

wherein R¹¹ and r are as defined above,
is preferred.

As the aforementioned (e) hydrocarbon compound having one reactive unsaturated group in one molecule, a monounsaturated hydrocarbon having 9 to 30 carbon atoms is preferred, and a 1-alkene is more preferable. As examples of 1-alkene, mention may be made of 1-nonene, 1-decene, 1-undecene, 1-dodecene, 1-tridecene, 1-tetradecene and the like. As examples of the aforementioned linear organopolysiloxane having one reactive unsaturated group in one molecule, mention may be made of a dimethylpolysiloxane in which one terminal is capped by a vinyl group, a methylphenylpolysiloxane in which one terminal is capped by a vinyl group, and the like.

The hydrosilylation reaction is preferably carried out in the presence of a catalyst. As examples of the catalyst, mention may be made of a compound such as platinum, ruthenium, rhodium, palladium, osmium, iridium or the like. A platinum compound is, in particular, effective since the catalytic activity thereof is high. As examples of platinum compounds, mention may be made of chloroplatinic acid; platinum metal; a platinum metal-supported carrier such as platinum-supported alumina, platinum-supported silica, platinum-supported carbon black or the like; and a platinum complex such as platinum-vinylsiloxane complex, platinum phosphine complex, platinum-phosphite complex, platinum alcholate catalyst or the like. The usage amount of the catalyst may range from 0.5 to 1,000 ppm as a platinum metal in the case of using a platinum catalyst.

In addition, the aforementioned sugar alcohol-modified silicone may be subjected to a hydrogenation treatment in order to ameliorate odor after the reaction due to the residual unsaturated compound. For the hydrogenation treatment, there are a method using a pressurized hydrogen gas and a method using a hydrogen adding agent such as a metal hydride or the like. In addition, in the aforementioned hydrogenation treatment, there are a homogeneous reaction and a heterogeneous reaction. One of these reactions can also be carried out, and the reactions can also be carried out in combination. Considering an advantage in that the used catalyst does not remain in a product, a heterogeneous catalytic hydrogenation reaction using a solid catalyst is most preferable.

As the solid catalyst (hydrogenation catalyst), a common noble metal-based catalyst such as a platinum-based catalyst, a palladium-based catalyst or the like, and a nickel-based catalyst can be used. More particular, as examples thereof, mention may be made of an elemental substance such as nickel, palladium, platinum, rhodium, cobalt or the like, and a catalyst of a combination of plural metals such as platinum-palladium, nickel-copper-chromium, nickel-copper-zinc, nickel-tungsten, nickel-molybdenum or the like. As examples of a catalyst carrier optionally used, mention may be made of activated carbon, silica, silica alumina, alumina, zeolite and the like. In addition, a copper-containing hydrogenation catalyst such as Cu—Cr, Cu—Zn, Cu—Si, Cu—Fe—Al, Cu—Zn—Ti and the like may be mentioned. The form of the aforementioned hydrogenation catalyst cannot be completely determined since the form may vary depending on the type of reactor, and can be appropriately selected from powders, granules, tablets and the like. In addition, the platinum catalyst used in the synthesis step (hydrosilylation reaction) can also be used as it is. The aforementioned hydrogenation catalyst can be used alone or in combination with two or more types thereof.

The hydrogenation treatment can also be used in order to purify a crude product of the aforementioned sugar alcohol-modified silicone obtained by the aforementioned addition reaction. More particularly, the aforementioned purification can be carried out by deodorization due to the hydrogenation treatment in a solvent or without a solvent in the presence of a hydrogenation catalyst. The aforementioned purified product can preferably be used in a cosmetic in which reduction of odor and miscibility with other cosmetic components are desired. In addition, as the pre-step or post-step of the aforementioned deodorization, a stripping treatment in which light products are removed by distillation by contacting a nitrogen gas with respect to a crude product or a hydrogenated product of a sugar alcohol-modified silicone can preferably be carried out. In the aforementioned hydrogenation treatment, solvents, reaction conditions, pressure-reduction conditions and the like used in purification of conventional organopolysiloxane copolymers or polyether-modified silicones can be applied and selected without any restrictions.

Alternatively, the odor of the crude product of the aforementioned sugar alcohol-modified silicone obtained by the aforementioned addition reaction can also be easily reduced by carrying out a stripping step in which light products are removed by distillation by contacting a nitrogen gas under reduced pressure after an unreacted unsaturated compound is hydrolyzed by adding an acid substance. The effects of reducing the odor of the sugar alcohol-modified silicone in accordance with the present invention are superior, and even if other modified silicones are subjected to the acid treatment in the same manner as that of the present invention, similar effects of reducing the odor to those obtained in the present invention cannot be obtained. Even in the aforementioned other modified silicones, if they are subjected to a hydrogenation treatment, the effects of reducing the odor obtained in the present invention may be obtained. However, the steps of the aforementioned hydrogenation treatment are complicated, and relatively expensive reagents and a specific apparatus are required. In contrast, in the present invention, since it is not necessary to carry out the aforementioned hydrogenation treatment, the present invention has an advantage in industrial scale operations, and the deodorized sugar alcohol-modified silicone or a composition containing the same can be easily provided at low cost. The acid substance is not particularly restricted, any one defined as a Lewis acid, a Bronsted acid, or an Arrhenius acid may be used. The acid substance used in the present invention is preferably a water-soluble acid. Therefore, the acid substance used in the present invention is preferably an Arrhenius acid releasing protons in an aqueous solution. The acid substance can be used alone or in combination with two or more types thereof. In the present invention, by use of the aforementioned acid substance, the aforementioned sugar alcohol-modified silicone can be substantially deodorized without the chemical bond-breaking of carbon-oxygen bonds or silicon-oxygen bonds, and odor production can be almost completely controlled over time.

The aforementioned acid substance can be selected from the group consisting of inorganic acids, organic acids, acidic inorganic salts, solid acids, and acidic platinum catalysts.

The inorganic acids are not particularly restricted. As examples thereof, mention may be made of, for example, hydrochloric acid, sulfuric acid, nitric acid, phosphoric acid, carbonic acid, boric acid, sulfonic acid, sulfinic acid and the like. One including an organic group such as benzenesulfonic acid or the like is not preferred as the inorganic acid.

The organic acids are not particularly restricted, and a monocarboxylic acid such as a monohydroxymonocarboxylic acid or a dihydroxymonocarboxylic acid, a dicarboxylic acid such as a monohydroxydicarboxylic acid or a dihydroxydicarboxylic acid, a polycarboxylic acid or the like can be used. As examples thereof, mention may be made of, linear saturated aliphatic monocarboxylic acids (alkanoic acids) such as formic acid, acetic acid, trifluoroacetic acid, propionic acid, butyric acid, valeric acid, capronic acid, enanthic acid, caprylic acid, pelargonic acid, capric acid, undecanoic acid and the like; branched saturated aliphatic monocarboxylic acids (alkanoic acids) such as 2-methylpropanoic acid, 2-methylbutanoic acid, trimethylpropanoic acid, 2-methylpentanoic acid, trimethylacetic acid and the like; unsaturated aliphatic monocarboxylic acids (alkenic acids) such as acrylic acid, methacrylic acid, crotonic acid, isocrotonic acid, acetovinylic acid, acetoallylic acid, hexenoic acid, heptenoic acid, octenoic acid and the like; unsaturated aliphatic monocarboxylic acids (alkynic acids) such as proriolic acid, tetrolic acid, allylacetic acid, hexynoic acid, octynoic acid and the like; polyvalent unsaturated aliphatic monocarboxylic acids such as pentadienoic acid, sorbic acid, and the like; alpha-hydroxymonocarboxylic acids such as citric acid, lactic acid, glycolic acid, alpha-oxybutyric acid and the like; beta-hydroxymonocarboxylic acids such as 2-hydroxyvaleric acid, 2-hydroxycaproic acid, beta-oxybutyric acid and the like; gamma-hydroxymonocarboxylic acids such as gamma-oxybutyric acid and the like; dihydroxymonocarboxylic acids such as glycelic acid and the like; other hydroxymonocarboxylic acids such as hydroxyl(meth)acrylic acids and the like; saturated aliphatic dicarboxylic acids such as oxalic acid, malonic acid, succinic acid, glutaric acid, adipic acid and the like; monohydroxy saturated aliphatic dicarboxylic acids such as tartronic acid, malic acid and the like; dihydroxy saturated aliphatic dicarboxylic acids such as tartaric acid and the like; unsaturated aliphatic dicarboxylic acids such as maleic acid, fumaric acid and the like; aromatic monocarboxylic acids such as benzoic acid and the like; aromatic dicarboxylic acids such as phthalic acid and the like; amino acids such as glycine, alanine, valine, leucine, glutamic acid, asparagic acid, PL-pyrrolidone carboxylic acid and the like; and polycarboxylic acids such as gallic acid and the like.

In addition, as the organic acid, an alkylsulfuric acid, an alkylphosphoric acid, phenol or the like can also be used. A higher fatty acid or a salt thereof is not preferred as the organic acid.

The acidic inorganic salts are not particularly restricted, and are preferably water soluble. In particular, a water-soluble acidic inorganic salt is preferred, which is a solid at 25° C., and has a pH of an aqueous solution at 25° C. obtained by dissolving 50 g thereof in 1 L of ion-exchanged water, of 4 or less, preferably 3.5 or less, and more preferably 2.0 or less. In the case in which the acidic inorganic salt is a solid at room temperature (25° C.), it can be easily removed by filtration, if necessary. In addition, in the case in which the acidic inorganic salt is water soluble, it can be easily rinsed off with water. The pH value in the present invention is a value obtained by measuring an aqueous solution of a sample at room temperature (25° C.) by means of a pH meter equipped with a glass electrode.

As the acidic inorganic salt, for example, an acidic inorganic salt in which at least one hydrogen atom of an inorganic acid with two or more valences is neutralized by a base can be used. As examples of the inorganic acids with two or more valences, mention may be made of sulfuric acid, sulfurous acid and the like. As examples of the base, mention may be made of alkali metals, ammonia and the like.

The acidic inorganic salt is preferably one or more types of acidic inorganic salts comprising a hydrogensulfonic acid ion (HSO₄⁻) or a hydrogensulfurous acid ion (HSO₃⁻) and a monovalent cation (M⁺). As examples of the monovalent cation (M⁺), mention may be made of an alkali metal ion or an ammonium ion. One or more types of monovalent cations selected from the group consisting of sodium ions, potassium ions and ammonium ions are particularly preferred.

As examples of acidic inorganic salts, mention may be made of, for example, lithium hydrogensulfate, sodium hydrogensulfate, potassium hydrogensulfate, rubidium hydrogensulfate, cesium hydrogensulfate, ammonium hydrogensulfate, sodium hydrogensulfite, and hydrates thereof, as well as, Lewis acids such as AlCl₃, FeCl₃, TiCl₄, BF₃.Et₂O and the like. The pH of an aqueous solution obtained by dissolving 50 g of the acidic inorganic salt in 1 L of ion-exchanged water is shown in the following table. In view of technological effects of reducing odor, as the water-soluble acidic inorganic salt with a pH of 2.0 or less, use of one or more types of acidic inorganic salts selected from the group consisting of sodium hydrogensulfate, potassium hydrogensulfate and ammonium hydrogensulfate is most preferable.

TABLE 1
Table 1

| Acidic inorganic salt | pH (50 g/L) |
|---|---|
| Sodium hydrogensulfate | 1.5 or less |
| Potassium hydrogensulfate | 2.0 or less |
| Ammonium hydrogensulfate | 1.5 or less |
| Sodium hydrogensulfite | 3.5 |

As the solid acid, for example, an acidic solid substance such as activated white earth, acid earth, solid acidic zirconium oxide, strong acidic cation-exchange resin, fluorinated sulfonic acid resin, alumina, silica alumina, zeolite and the like can be used. A solid acidic zirconium oxide is preferred. As examples of solid acidic zirconium oxide, mention may be made of, for example, a solid acidic zirconium prepared by treating zirconium hydroxide with sulfuric acid, followed by baking at 300° C. or more, more particularly, a solid acidic zirconium prepared by burning a molded product obtained by kneading and molding aluminum hydroxide or hydrous oxide, zirconium hydroxide or hydrous oxide, and a compound containing a sulfuric acid component, at a temperature at which zirconia having a tetragonal structure can be obtained, more particularly at 300° C. or more, and more particularly zirconia sulfate and the like. As solid acidic zirconium oxide, SZA-60 manufactured by JX Nippon Oil & Energy Corporation is commercially available. The strong acidic cation-exchange resin is, for example, a cation exchange resin in which the functional group is a sulfonic acid group (—SO₃H), and as commercially available products thereof, there are Amberlyst 15, Amberlyst 16, Amberlyst 31, and Amberlyst 35, sold by Organo Corporation, and the like. The fluorinated sulfonic acid resin is a perfluorinated polymer having a sulfonic acid group in a pendant form, binding to a polymer chain, and as examples thereof, mention may be made of those described in Japanese Examined Patent Application, Second Publication No. S59-4446, and the like.

As the acid platinum catalyst, chloroplatinic acid, an alcohol-modified chloroplatinic acid, an olefin complex of chloroplatinic acid, a ketone complex of chloroplatinic acid, a vinylsiloxane complex of chloroplatinic acid, platinum tetrachloride or the like can be used. Chloroplatinic acid is preferred.

The aforementioned acid treatment step can be carried out by contacting the aforementioned sugar alcohol-modified silicone with the aforementioned acid substance in any mode.

More particularly, the aforementioned acid treatment step can be carried out, for example, by operations of adding at least one type of the aforementioned acid substances and optionally adding water or an organic solvent such as alcohol, in a reaction system (for example, a reaction vessel such as a flask) containing the aforementioned sugar alcohol-modified silicone, and stirring the mixture.

In particular, preferably, at least one type of the aforementioned acid substances and water are added in a reaction system containing the aforementioned sugar alcohol-modified silicone, followed by carrying out a stirring and mixing treatment by means of mechanical force under heating. In addition, the aforementioned treatment is preferably carried out under the co-presence of a solvent such as a lower monovalent alcohol or the like. The acid treatment step can be carried out by freely selecting the temperature and the treatment period, and can be carried out at a temperature ranging from 0 to 200° C. and more preferably ranging from 50 to 100° C. in a reaction period ranging from 0.5 to 24 hours and more preferably ranging from about one hour to 10 hours. The usage amount of the acid substance can be appropriately selected in accordance with the acid strength, the treatment apparatus, the treatment period and the treatment temperature. For example, in the case of an acid substance with medium acid strength, such as sodium hydrogensulfate, potassium hydrogensulfate, ammonium hydrogensulfate, citric acid, glycolic acid, phosphoric acid or the like, the amount of the acid substance preferably ranges from 10 to 500 ppm, and more preferably ranges from to 200 ppm, with respect to the amount of the sugar alcohol-modified silicone. In addition, in the case of an acid substance with increased acid strength, such as hydrochloric acid, sulfuric acid, or the like, the amount of the acid substance preferably ranges from 0.1 to 50 ppm with respect to the amount of the sugar alcohol-modified silicone. In the case of a weak acid substance with reduced acid strength or a solid acid represented by activated white earth, acid earth, solid acidic zirconium oxide, strong acidic cation-exchange resin, fluorinated sulfonic acid resin, zeolite and the like, the amount of the acid substance preferably ranges from 500 to 10,000 ppm with respect to the amount of the sugar alcohol-modified silicone.

The method for manufacturing the aforementioned sugar alcohol-modified silicone preferably includes a step of heating and/or reducing the pressure (stripping step), after the aforementioned acid treatment step. By the aforementioned heating and/or reducing of the pressure, components with low boiling points, which are substances causing odor, can be removed (stripped). In addition, by carrying out the acid treatment step again after the stripping step, the substances causing odor can be removed much more. At this time, there is an advantage in that in the case of the acid substance remaining in the reaction system, it is not necessary to newly add the acid substance, and only water may be added thereto. Namely, the aforementioned acid treatment step and stripping step can be repeated respectively two or more times in order to enhance the degree of deodorization.

The “components with low boiling points” removed by the stripping step may be volatile components such as reaction solvents used in synthesis of the aforementioned sugar alcohol-modified silicone and the like, in addition to the carbonyl compounds such as propionaldehyde which may be believed as a substance causing odor.

The stripping step may be carried out before the aforementioned acid treatment step.

In the stripping method, conventional reaction conditions may be applied. The stripping step is carried out preferably under normal pressure or under reduced pressure and preferably at 120° C. or less. In order to efficiently carry out the stripping step, the step is preferably carried out under reduced pressure or under steam of an inert gas such as nitrogen gas or the like. In an example of the step of removing the components with low boiling points, the sugar alcohol-modified silicone containing the components with low boiling points or the composition thereof or the hydrogen additive thereof is placed in a flask equipped with a reflex condenser, a nitrogen introducing port and the like, and the flask is heated under reduced pressure while supplying nitrogen gas, to maintain a constant level of pressure and the temperature, and thereby, remove light products. In general, the reduced pressure used herein ranges from 0.1 to 10.0 KPa, the heating temperature ranges from 50 to 170° C., and the reaction period ranges from 10 minutes to 24 hours.

In the present invention, after the aforementioned acid treatment step, the reaction system containing the aforementioned sugar alcohol-modified silicone may be subjected to a neutralization treatment with a basic substance. The basic substance may be used alone or in combination with two or more types thereof. As examples of the basic substances, mention may be made of inorganic bases such as sodium hydroxide, potassium hydroxide, calcium hydroxide, barium hydroxide, aqueous ammonia, sodium hydrogencarbonate and the like, organic bases such as amine, pyridine and the like, and the like. The amount of the basic substance is preferably for neutralizing the reaction system containing the aforementioned sugar alcohol-modified silicone, and can also be adjusted, if necessary, so that the reaction system becomes weakly acidic or weakly basic.

In the present invention, before and/or after the aforementioned acid treatment step, or before and/or after the aforementioned stripping step, a hydrogenation treatment may be carried out. If a deodorization treatment is carried out by the hydrogenation reaction, a sufficient effect of reducing odor can be obtained. However, in the hydrogenation treatment, the steps are complicated, and relatively expensive reagents and a specific apparatus are required. On the other hand, in the present invention, a sufficient effect of reducing odor can be obtained by the aforementioned acid treatment step, and for this reason, it is not necessary to carry out the aforementioned hydrogenation treatment. Therefore, in the present invention, the hydrogenation treatment can be omitted.

The aforementioned sugar alcohol-modified silicone (hereinafter, referred to as “(A) sugar alcohol-modified silicone”) possesses a specified hydrophilic group, and can provide, as an oil agent component of a cosmetic for hair of the present invention, smooth combability with fingers without a frictional sensation during wetting and during drying to the hair. Similarly, superior foaming properties and a superior feeling on touch of foam are exhibited, smooth combability with a comb or fingers during drying and a moisturizing feeling on touch are exhibited without an uncomfortable sticky sensation, and a flexible styling sensation can be provided to the hair. In addition, superior durability can be provided. Furthermore, since the aforementioned (A) sugar alcohol-modified silicone possesses superior miscibility with each component in the cosmetic for hair, increased stability can be provided to the cosmetic for hair of the present invention.

The blending amount of the aforementioned (A) sugar alcohol-modified silicone contained in the cosmetic for hair of the present invention is not particularly restricted, and for example, can range from 0.0001 to 20% by weight (mass), can preferably range from 0.001 to 10% by weight (mass) and in particular, can preferably range from 0.01 to 5% by weight (mass).

The cosmetic for hair of the present invention can be appropriately prepared by mixing the aforementioned (A) sugar alcohol-modified silicone with various conventional components known in the field of cosmetics. Hereinafter, various conventional components are described in detail.

Oil Agent

The cosmetic for hair of the present invention preferably comprises (B) an oil agent. Use of the aforementioned (A) sugar alcohol-modified silicone together with the aforementioned (B) oil agent can achieve, for example, improvements of a feeling on touch which can be difficultly achieved by using a conventional polyglycerol-modified silicone with an oil agent. The “oil agent” in the present invention is generally used as a component of a cosmetic, and is not particularly restricted. The aforementioned (B) oil agent is usually in the form of a liquid at 5° C. to 100° C., and may be in the form of a solid such as a wax or in the form of a gum or a paste which has an increased viscosity and is thickened, as described below. The aforementioned (B) oil agent can be used as a single type thereof or in combination with two or more types thereof, in accordance with the purpose thereof.

The aforementioned (B) oil agent is preferably at least one type selected from (B1) a silicone-based oil agent and (B2) a non-silicone-based oil agent selected from organic oils. The types, viscosities and the like of the aforementioned oil agents can be appropriately selected in accordance with types and usages of cosmetics for hair.

The aforementioned (B1) silicone-based oil agent is generally hydrophobic, and the molecular structure thereof may be a cyclic, linear or branched structure. The functional groups of the silicone-based oils are generally an alkyl group such as a methyl group, a phenyl group or a hydroxyl group. An organo-modified silicone in which a part or all of the aforementioned functional groups is/are substituted with functional groups may be used. The aforementioned organo-modified silicone is an organo-modified silicone other than the aforementioned (A) sugar alcohol-modified silicone, and is a component to be blended in a cosmetic for hair. The organo-modified silicone may have an alkylene chain, an aminoalkylene chain or a polyether chain in addition to the polysiloxane bond as a main chain, and may comprise a so-called block copolymer. In addition, the aforementioned organo-modified group may be present at one or both of the terminals of the side chain of the polysiloxane chain. More particularly, as examples thereof, mention may be made of amino-modified silicones, aminopolyether-modified silicones, epoxy-modified silicones, carboxyl-modified silicones, amino acid-modified silicones, acryl-modified silicones, phenol-modified silicones, amidoalkyl-modified silicones, polyamide-modified silicones, aminoglycol-modified silicones, alkoxy-modified silicones, C8-30 higher alkyl-modified silicones, and alkyl-modified silicone resins.

As the linear organopolysiloxanes, organopolysiloxanes represented by the following general formula (5):

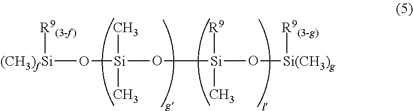

wherein
R⁹ is a hydrogen atom, or a group selected from a hydroxyl group, a substituted or non-substituted monovalent hydrocarbon group, an alkoxy group, a polyoxyalkylene group, and a polyorganosiloxane group; each of f and g

independently denotes an integer ranging from 0 to 3; g′ is an integer ranging from 0 to 10,000; and 1′ is an integer ranging from 0 to 10,000, with the proviso that 1≦g′+l′≦10,000,

can be used. The viscosity of the linear organopolysiloxanes at 25° C. is not particularly restricted, and may usually range from 0.65 to 1,000,000 mm²/sec, which corresponds to the viscosity of a so-called silicone oil. On the other hand, the organopolysiloxane may have an ultra high viscosity which corresponds to that of a silicone gum.

As examples of substituted or non-substituted monovalent hydrocarbon groups, mention may be made of linear or branched alkyl groups having 1 to 30 carbon atoms such as a methyl group, an ethyl group, a propyl group, a butyl group, a pentyl group, a hexyl group, a heptyl group, an octyl group, a decyl group, a dodecyl group and the like; cycloalkyl groups having 3 to 30 carbon atoms such as a cyclopentyl group, a cyclohexyl group and the like; aryl groups having 6 to 30 carbon atoms such as a phenyl group, a tolyl group, a xylyl group, a naphthyl group and the like; and substituted groups thereof, in which hydrogen atoms binding to carbon atoms of the aforementioned groups are at least partially substituted by a halogen atom such as a fluorine atom, or an organic group such as an epoxy group, an acyl group, a carboxyl group, an amino group, an amide group, a (meth)acryl group, a mercapto group, a carbinol group, a phenol group or the like. As examples of alkoxy groups, mention may be made of an alkoxy group having 1 to carbon atoms such as a methoxy group, an ethoxy group, a propoxy group or the like.

As examples of silicone oils, mention may be made of, for example, a dimethylpolysiloxane in which both molecular terminals are capped with trimethylsiloxy groups (dimethylsilicone with a low viscosity such as 2 mPa·s or 6 mPa·s to dimethylsilicone with a high viscosity such as 1,000,000 mPa·s, and in addition, a dimethylsilicone with an ultra-high viscosity), an organohydrogenpolysiloxane, a methylphenylpolysiloxane in which both molecular terminals are capped with trimethylsiloxy groups, a copolymer of methylphenylsiloxane and dimethylsiloxane in which both molecular terminals are capped with trimethylsiloxy groups, a diphenylpolysiloxane in which both molecular terminals are capped with trimethylsiloxy groups, a copolymer of diphenylsiloxane and dimethylsiloxane in which both molecular terminals are capped with trimethylsiloxy groups, a trimethylpentaphenyltrisiloxane, a phenyl(trimethylsiloxy)siloxane, a methylalkylpolysiloxane in which both molecular terminals are capped with trimethylsiloxy groups, a copolymer of methylalkylsiloxane and dimethylpolysiloxane in which both molecular terminals are capped with trimethylsiloxy groups, a copolymer of methyl(3,3,3-trifluoropropyl)siloxane and dimethylsiloxane in which both molecular terminals are capped with trimethylsiloxy groups, an α,ω-diethoxypolydimethylsiloxane, a higher alkoxy-modified silicone, a higher fatty acid-modified silicone, dimethiconol, a siloxane with a low molecular weight such as a 1,1,1,3,5,5,5-heptamethyl-3-octyltrisiloxane, a 1,1,1,3,5,5,5-heptamethyl-3-dodecyltrisiloxane, a 1,1,1,3,5,5,5-heptamethyl-3-hexadecyltrisiloxane, a tristrimethylsiloxymethylsilane, a tristrimethylsiloxyalkylsilane, a tetrakistrimethylsiloxysilane, a tetramethyl-1,3-dihydroxydisiloxane, an octamethyl-1,7-dihydroxytetrasiloxane, a hexamethyl-1,5-diethoxytrisiloxane, a hexamethyldisiloxane, an octamethyltrisiloxane, or the like, a dimethylpolysiloxane in which both molecular terminals are capped with trimethylsilyl groups, an α,ω-dihydroxypolydimethylsiloxane, and the like.

In the cosmetic for hair of the present invention, a so-called silicone gum having 1,000,000 mm²/s or more, which has ultra-high viscosity but possesses fluidity, can also be preferably used as a silicone oil. The silicone gum is a linear diorganopolysiloxane having an ultra-high degree of polymerization, and is also referred to as a silicone raw rubber or an organopolysiloxane gum. The silicone gum possesses a high degree of polymerization, and for this reason, it has a measurable degree of plasticity. In view of this, the silicone gum is different from the aforementioned oil silicones. The aforementioned silicone gum can be blended in the cosmetic for hair according to the present invention as it is, or as a liquid gum dispersion (an oil dispersion of the silicone gum) in which the silicone gum is dispersed in an oil silicone.

As examples of the aforementioned silicone raw rubber, mention may be made of substituted or non-substituted organopolysiloxanes having a dialkylsiloxy unit (D unit) such as dimethylpolysiloxane, methylphenylpolysiloxane, aminopolysiloxane, methylfluoroalkyl polysiloxane and the like, or those having a slightly-crosslinking structure thereof and the like. As representative examples thereof, there are those represented by the following general formula:

R¹⁰(CH₃)₂SiO{(CH₃)₂SiO}ₛ{(CH₃)R¹²SiO}ₜSi(CH₃)₂R¹⁰

wherein R¹² is a group selected from a vinyl group, a phenyl group, an alkyl group having 6 to 20 carbon atoms, an aminoalkyl group having 3 to 15 carbon atoms, a perfluoroalkyl group having 3 to 15 carbon atoms, and a quaternary ammonium salt group-containing alkyl group having 3 to 15 carbon atoms; the terminal group R¹⁰ is a group selected from an alkyl group having 1 to 8 carbon atoms, a phenyl group, a vinyl group, an aminoalkyl group having 3 to carbon atoms, a hydroxyl group and an alkoxy group having 1 to 8 carbon atoms; s=2,000 to 6,000; t=0 to 1,000; and s+t=2,000 to 6,000. Among these, a dimethylpolysiloxane raw rubber having a degree of polymerization ranging from 3,000 to 20,000 is preferred. In addition, an amino-modified methylpolysiloxane raw rubber having a 3-aminopropyl group, an N-(2-aminoethyl)-3-aminopropyl group or the like on the side chain or the terminal of the molecule is preferred. In addition, in the present invention, the silicone gum can be used alone or in combination with two or more types thereof, as necessary.

The silicone gum has an ultra-high degree of polymerization. For this reason, the silicone gum can exhibit a superior retention property on hair or skin, and can form a protective film with a superior aeration property. For this reason, the silicone gum is a component which can particularly provide glossiness and luster on hair and can impart a texture with tension on the entire hair during use and after use.

The blending amount of the silicone gum may range from 0.05 to 30% by weight (mass) and may preferably range from 1 to 15% by weight (mass), with respect to the total amount of the cosmetic for hair. When the silicone gum is used as an emulsion composition prepared via a step of preliminarily emulsifying (including emulsion polymerization), the silicone gum can be easily blended, and can stably be blended in the cosmetic for hair of the present invention. If the blending amount of the silicone gum is below the aforementioned lower limit, an effect of imparting a specific feeling on touch or glossiness with respect to hair may be insufficient.

As cyclic organopolysiloxanes, for example, organopolysiloxanes represented by the following general formula (6):

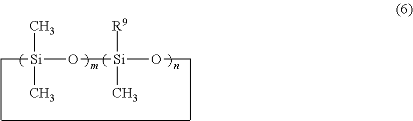

wherein
R⁹ is the same as defined above;
m is an integer ranging from 0 to 8; and
n is an integer ranging from 0 to 8, with the proviso that 3≦m+n≦8,
can be used.

As examples of cyclic organopolysiloxanes, mention may be made of hexamethylcyclotrisiloxane (D3), octamethylcyclotetrasiloxane (D4), decamethylcyclopentasiloxane (D5), dodecamethylcyclohexasiloxane (D6), 1,1-diethylhexamethylcyclotetrasiloxane, phenylheptamethylcyclotetrasiloxane, 1,1-diphenylhexamethylcyclotetrasiloxane, 1,3,5,7-tetravinyltetramethylcyclotetrasiloxane, 1,3,5,7-tetramethylcyclotetrasiloxane, 1,3,5,7-tetracyclohexyltetramethylcyclotetrasiloxane, tris(3,3,3-trifluoropropyl)trimethylcyclotrisiloxane, 1,3,5,7-tetra(3-methacryloxypropyl)tetramethylcyclotetrasiloxane, 1,3,5,7-tetra(3-acryloxypropyl)tetramethylcyclotetrasiloxane, 1,3,5,7-tetra(3-carboxypropyl)tetramethylcyclotetrasiloxane, 1,3,5,7-tetra(3-vinyloxypropyl)tetramethylcyclotetrasiloxane, 1,3,5,7-tetra(p-vinylphenyl)tetramethylcyclotetrasiloxane, 1,3,5,7-tetra[3-(p-vinylphenyl)propyl]tetramethylcyclotetrasiloxane, 1,3,5,7-tetra(N-acryloyl-N-methyl-3-aminopropyl)tetramethylcyclotetrasiloxane, 1,3,5,7-tetra(N,N-bis(lauroyl)-3-aminopropyl) tetramethylcyclotetrasiloxane and the like.

As branched organopolysiloxanes, for example, organopolysiloxanes with a low molecule having volatility represented by the following general formula (7):

R⁹₍₄₋ₚ₎Si(OSiCH₃)_{q} (7)

wherein
R⁹ is the same as defined above;
p is an integer ranging from 1 to 4; and
q is an integer ranging from 0 to 500,
and so-called silicone resins in the form of a liquid, a solid or the like can be used.

As branched organopolysiloxanes, mention may be made of a siloxane with a low molecule such as methyltristrimethylsiloxysilane, ethyltristrimethylsiloxysilane, propyltristrimethylsiloxysilane, tetrakistrimethylsiloxysilane, phenyltristrimethylsiloxysilane or the like; or a silicone resin of a highly branched molecular structure, a net-like molecular structure or a cage-like molecular structure may be used. A silicone resin containing at least a monoorganosiloxy unit (T unit) and/or a siloxy unit (Q unit) is preferred. The aforementioned silicone resins having branched units possess a net-like structure. In the case of applying the silicone resins to hair or the like, a uniform film is formed and protective effects with respect to dryness and low temperature are provided. In addition, the silicone resins having branched units tightly adhere to hair or the like, and can provide glossiness and a transparent impression to hair or the like.

Hereinafter, a higher alkyl-modified silicone, an alkyl-modified silicone resin and a polyamide-modified silicone resin which are particularly preferred as the organo-modified silicones are described. The higher alkyl-modified silicone is in the form of a wax at room temperature, and is a component useful as a part of a base material of an oil-based solid cosmetic for hair. Therefore, the higher alkyl-modified silicones can be preferably used in the cosmetics for hair of the present invention. As examples of the aforementioned higher alkyl-modified silicone waxes, mention may be made of a methyl(long chain alkyl)polysiloxane having both molecular terminals capped with trimethylsiloxy groups, a copolymer of a dimethylpolysiloxane and a methyl(long chain alkyl)siloxane having both molecular terminals capped with trimethylsiloxy groups, a dimethylpolysiloxane modified with long chain alkyls at both terminals, and the like. As examples of commercially available products thereof, mention may be made of, AMS-C30 Cosmetic Wax, 2503 Cosmetic Wax and the like (manufactured by Dow Corning Corporation, in the USA).

The aforementioned (A) sugar alcohol-modified silicone exhibits a superior dispersion property of a higher alkyl-modified silicone wax, and for this reason, a cosmetic for hair exhibiting superior storage stability for a long time can be obtained. In addition, a superior forming property of the cosmetic for hair can also be exhibited. In particular, in a system containing powder(s), there is an advantage in that separation of the higher alkyl-modified silicone wax hardly occurs, and an oil-based cosmetic for hair which can exhibit superior form-retaining strength and can be smoothly and uniformly spread during application can be provided.

In the cosmetic for hair of the present invention, the higher alkyl-modified silicone wax preferably has a melting point of 60° C. or higher in view of a cosmetic durability effect and stability at increased temperatures.

The alkyl-modified silicone resin is a component for imparting sebum durability, a moisture-retaining property, and a fine texture feeling on touch to the cosmetic for hair, and one in the form of a wax at room temperature can be preferably used. For example, a silsesquioxane resin wax described in Published Japanese Translation No. 2007-532754 of the PCT International Application may be mentioned. As commercially available products thereof, SW-8005 C30 RESIN WAX (manufactured by Dow Corning Corporation in the USA) and the like may be mentioned.

The aforementioned (A) sugar alcohol-modified silicone can uniformly disperse the alkyl-modified silicone resin wax in the cosmetic for hair, in the same manner as described for the higher alkyl-modified silicone wax. In addition, an oil phase containing the aforementioned alkyl-modified silicone resin wax can be stably emulsified by optionally using together with the other surfactant. A conditioning effect with respect to hair can be improved and a fine texture and moisturized feeling on touch can be imparted.

As examples of polyamide-modified silicones, mention may be made of, for example, siloxane-based polyamide compounds described in U.S. Pat. No. 5,981,680 (Japanese Unexamined Patent Application, First Publication No. 2000-038450) and Published Japanese Translation No. 2001-512164 of the PCT International Application. As examples of commercially available products, mention may be made of 2-8178 Gellant, 2-8179 Gellant and the like (manufactured by Dow Corning Corporation, in the USA). The aforementioned polyamide-modified silicones are also useful as an oil-based raw material, and in particular, a thickening/gelling agent of a silicone oil.

In the case of using the polyamide-modified silicone together with the aforementioned (A) sugar alcohol-modified silicone, the cosmetic for hair of the present invention can exhibit a good spreading property, a good styling property, a superior stable sensation and a superior adhesive property in the case of applying to hair or the like. In addition, there are advantages in view of qualities in that a glossy transparent sensation and superior glossiness can be provided, the viscosity or hardness (flexibility) of the whole cosmetic for hair containing oil-based raw material(s) can be appropriately adjusted, and an oily sensation (oily and sticky feeling on touch) can be totally controlled. In addition, by use of the aforementioned (A) sugar alcohol-modified silicone, dispersion stability of perfume(s), powder(s) and the like can be improved. For this reason, for example, there is a characteristic in that a uniform and fine cosmetic sensation can be maintained for a long time.

As the aforementioned (B2) organic oil agent, (B2-1) a higher alcohol, (B2-2) a hydrocarbon oil, (B2-3) a fatty acid ester oil, and (B2-4) a higher fatty acid, fats and oils, or a fluorine-based oil agent are representative. In the present invention, the aforementioned (B2) organic oil agent is not particularly restricted, but a higher alcohol, a hydrocarbon oil, a fatty acid ester oil and a higher fatty acid are preferred. The aforementioned oil agents can exhibit superior miscibility and dispersibility with respect to the aforementioned (A) sugar alcohol-modified silicone. For this reason, they can be stably blended in a cosmetic composition for hair of the present invention, and they can supplement effects of the aforementioned (A) sugar alcohol-modified silicone and strengthen the inherent effects of each of the aforementioned components (A) and (B2).

The aforementioned (B2-1) higher alcohol is, for example, a higher alcohol having 10 to 30 carbon atoms. The aforementioned higher alcohol is a saturated or unsaturated monovalent aliphatic alcohol, and the moiety of the hydrocarbon group thereof may be linear or branched, but a linear one is preferred. As examples of higher alcohols having 10 to 30 carbon atoms, mention may be made of lauryl alcohol, myristyl alcohol, palmityl alcohol, stearyl alcohol, behenyl alcohol, hexadecyl alcohol, oleyl alcohol, isostearyl alcohol, hexyldodecanol, octyldodecanol, cetostearyl alcohol, 2-decyltetradecinol, cholesterol, sitosterol, phytosterol, lanosterol, lanolin alcohol, hydrogenated lanolin alcohol and the like. In the present invention, use of a higher alcohol having a melting point ranging from 40 to 80° C. or use of a combination of plural higher alcohols so as to have a melting point thereof ranging from 40 to 70° C. is preferred. The aforementioned higher alcohols can form an aggregate which is a so-called alpha gel, together with a surfactant. Thereby, the higher alcohols may possess a function of increasing viscosity of a preparation, and stabilize an emulsion. For this reason, they are, in particular, useful as a base agent of a cosmetic for hair.

As examples of the aforementioned (B2-2) hydrocarbon oils, mention may be made of liquid paraffin, light liquid isoparaffin, heavy liquid isoparaffin, vaseline, n-paraffin, isoparaffin, isododecane, isohexadecane, polyisobutylene, hydrogenated polyisobutylene, polybutene, ozokerite, ceresin, microcrystalline wax, paraffin wax, polyethylene wax, polyethylene/polypropylene wax, squalane, squalene, pristane, polyisoprene and the like.

As examples of the aforementioned (B2-3) fatty acid ester oils, mention may be made of hexyldecyl octanoate, cetyl octanoate, isopropyl myristate, isopropyl palmitate, butyl stearate, hexyl laurate, myristyl myristate, oleyl oleate, decyl oleate, octyldodecyl myristate, hexyldecyl dimethyloctanoate, cetyl lactate, myristyl lactate, diethyl phthalate, dibutyl phthalate, lanolin acetate, ethylene glycol monostearate, propylene glycol monostearate, propylene glycol dioleate, glyceryl monostearate, glyceryl monooleate, glyceryl tri-2-hexanoate, trimethylolpropane tri-2-ethylhexanoate, ditrimethylolpropane triethylhexanoate, ditrimethylolpropane isostearate/sebacate, trimethylolpropane trioctanoate, trimethylolpropane triisostearate, diisopropyl adipate, diisobutyl adipate, 2-hexyldecyl adipate, di-2-heptylundecyl adipate, diisostearyl malate, hydrogenated castor oil monoisostearate, N-alkylglycol monoisostearate, octyldodecyl isostearate, isopropyl isostearate, isocetyl isostearate, ethylene glycol di-2-ethylhexanoate, cetyl 2-ethylhexanoate, pentaerythritol tetra-2-ethylhexanoate, octyldodecyl gum ester, ethyl oleate, octyldodecyl oleate, neopentylglycol dicaprate, triethyl citrate, 2-ethylhexyl succinate, dioctyl succinate, isocetyl stearate, diisopropyl sebacate, di-2-ethylhexyl sebacate, diethyl sebacate, dioctyl sebacate, dibutyloctyl sebacate, cetyl palmitate, octyldodecyl palmitate, octyl palmitate, 2-ethylhexyl palmitate, 2-hexyldecyl palmitate, 2-heptylundecyl palmitate, cholesteryl 12-hydroxystearate, dipentaerythritol fatty acid ester, 2-hexyldecyl myristate, ethyl laurate, 2-octyldodecyl N-lauroyl-L-glutamate, di(cholesteryl/behenyl/octyldodecyl) N-lauroyl-L-glutamate, di(cholesteryl/octyldodecyl) N-lauroyl-L-glutamate, di(phytosteryl/behenyl/octyldodecyl) N-lauroyl-L-glutamate, di(phytosteryl/octyldodecyl) N-lauroyl-L-glutamate, isopropyl N-lauroylsarcosinate, diisostearyl malate, neopentylglycol dioctanoate, isodecyl neopentanoate, isotridecyl neopentanoate, isostearyl neopentanoate, isononyl isononanoate, isotridecyl isononanoate, octyl isononanoate, isotridecyl isononanoate, diethylpentanediol dineopentanoate, methylpentanediol dineopentanoate, octyldodecyl neodecanoate, 2-butyl-2-ethyl-1,3-propanediol dioctanoate, pentaerythrityl tetraoctanoate, pentaerythrityl hydrogenated rosin, pentaerythrityl triethylhexanoate, dipentaerythrityl (hydroxystearate/stearate/rosinate), polyglyceryl tetraisostearate, polyglyceryl-10 nonaisostearate, polyglyceryl-8 deca(erucate/isostearate/ricinoleate), (hexyldecanoic acid/sebacic acid) diglyceryl oligoester, glycol distearate (ethylene glycol distearate), diisopropyl dimer dilinoleate, diisostearyl dimer dilinoleate, di(isostearyl/phytosteryl) dimer dilinoleate, (phytosteryl/behenyl) dimer dilinoleate, (phytosteryl/isostearyl/cetyl/stearyl/behenyl) dimer dilinoleate, dimer dilinoleyl dimer dilinoleate, dimer dilinoleyl diisostearate, dimer dilinoleyl hydrogenated rosin condensate, dimer dilinoleic acid hardened castor oil, hydroxyalkyl dimer dilinoleyl ether, glyceryl triisooctanoate, glyceryl triisostearate, glyceryl trimyristate, glyceryl triisopalmitate, glyceryl trioctanoate, glyceryl trioleate, glyceryl diisostearate, glyceryl tri(caprylate/caprate), glyceryl tri(caprylate/caprate/myristate/stearate), hydrogenated rosin triglyceride (hydrogenated ester gum), rosin triglyceride (ester gum), glyceryl behenate eicosane dioate, glyceryl di-2-heptylundecanoate, diglyceryl myristate isostearate, cholesteryl acetate, cholesteryl nonanoate, cholesteryl stearate, cholesteryl isostearate, cholesteryl oleate, cholesteryl 12-hydroxystearate, cholesteryl ester of macadamia nut oil fatty acid, phytosteryl ester of macadamia nut oil fatty acid, phytosteryl isostearate, cholesteryl ester of soft lanolin fatty acid, cholesteryl ester of hard lanolin fatty acid, cholesteryl ester of long-chain branched fatty acid, cholesteryl ester of long-chain α-hydroxy fatty acid, octyldodecyl ricinoleate, octyldodecyl ester of lanolin fatty acid, octyldodecyl erucate, isostearic acid hardened castor oil, ethyl ester of avocado fatty acid, isopropyl ester of lanolin fatty acid, and the like. Lanolin and lanolin derivatives can also be used as the fatty acid ester oils.

As examples of the aforementioned (B2-4) higher fatty acids, mention may be made of, for example, lauric acid, myristic acid, palmitic acid, stearic acid, behenic acid, undecylenic acid, oleic acid, linolic acid, linolenic acid, arachidonic acid, eicosapentaenoic acid (EPA), docosahexaenoic acid (DHA), isostearic acid, 12-hydroxystearic acid, and the like.

As the aforementioned (B) oil agent, a silicone-based oil agent and a non-silicone-based oil agent may be used in combination. By use of the combination, in addition to a refreshing feeling on touch which the silicone oils inherently possess, the moisture of hair can be maintained and a moisturizing sensation such that hair is moisturized (also referred to as a moisturizing feeling on touch) or a smooth feeling on touch can be provided to the cosmetics for hair of the present invention. In addition, an advantage in that stability of the cosmetics over time is not impaired can be obtained. Furthermore, by a cosmetic comprising a hydrocarbon oil and/or a fatty acid ester oil and a silicone oil, the aforementioned moisturizing components (namely, the hydrocarbon oils and/or fatty acid ester oils) can be stably and uniformly applied on skin or hair. For this reason, effects of retaining moisture on the skin of the moisturizing components are improved. Therefore, a cosmetic comprising both a non-silicone-based oil agent and a silicone-based oil agent has an advantage in that a smoother and moisturizing feeling on touch can be provided, as compared with a cosmetic comprising only a non-silicone-based oil agent (such as a hydrocarbon oil, a fatty acid ester oil or the like).

In the present invention, in addition to the aforementioned oil agents, fats and oils, higher fatty acids, fluorine-based oils and the like may be used as the aforementioned (B) oil agents, and they may be used in combination of two or more types thereof. In particular, fats and oils derived from vegetables provide a healthy image derived from natural products and exhibit a superior moisture-retaining property and superior compatibility with hair. For this reason, they are preferably used in a cosmetic for hair of the present invention.

As examples of natural animal or vegetable fats and oils and semi-synthetic fats and oils, mention may be made of avocado oil, linseed oil, almond oil, ibota wax, perilla oil, olive oil, cacao butter, kapok wax, kaya oil, carnauba wax, liver oil, candelilla wax, beef tallow, hydrogenated beef tallow, apricot kernel oil, spermaceti wax, hydrogenated oil, wheat germ oil, sesame oil, rice germ oil, rice bran oil, sugar cane wax, sasanqua oil, safflower oil, shear butter, Chinese tung oil, cinnamon oil, jojoba wax, olive oil, squalane, shellac wax, turtle oil, soybean oil, tea seed oil, camellia oil, evening primrose oil, corn oil, lard, rapeseed oil, Japanese tung oil, rice bran wax, germ oil, horse fat, persic oil, palm oil, palm kernel oil, castor oil, hydrogenated castor oil, castor oil fatty acid methyl ester, sunflower oil, grape oil, bayberry wax, jojoba oil, hydrogenated jojoba ester, macadamia nut oil, beeswax, mink oil, cottonseed oil, cotton wax, Japanese wax, Japanese wax kernel oil, montan wax, coconut oil, hydrogenated coconut oil, tri-coconut oil fatty acid glyceride, mutton tallow, peanut oil, lanolin, liquid lanolin, reduced lanolin, lanolin alcohol, hard lanolin, lanolin acetate, lanolin fatty acid isopropyl ester, POE lanolin alcohol ether, POE lanolin alcohol acetate, lanolin fatty acid polyethylene glycol, POE hydrogenated lanolin alcohol ether, POE cholesterol ether, monostearyl glycerol ether (batyl alcohol), monooleyl glycerol ether (selachyl alcohol), egg yolk oil and the like, with the proviso that POE means polyoxyethylene.

As examples of fluorine-based oils, mention may be made of perfluoro polyether, perfluorodecalin, perfluorooctane and the like.

The blending amount of the aforementioned (B) oil agent in the cosmetic for hair of the present invention is not particularly restricted, and preferably ranges from 0.1 to 90% by weight (mass), more preferably ranges from 0.5 to 70% by weight (mass), furthermore preferably ranges from 1 to 50% by weight (mass), and in particular, preferably ranges from 5 to 25% by weight (mass).

In addition, the blending ratio between the aforementioned (B) oil agent and (A) sugar alcohol-modified silicone, namely the weight ratio of (B)/(A) preferably ranges from 0.01 to 100 and more preferably ranges from 0.1 to 50. If the blending amount of the aforementioned component (B) is increased too much, effects of the aforementioned component (A) may be reduced.

Surfactants

The cosmetic for hair of the present invention preferably comprises (C) a surfactant.

Types of the aforementioned (C) surfactants are not particularly restricted, and can be at least one type selected from the group consisting of (C1) anionic surfactants, (C2) cationic surfactants, (C3) nonionic surfactants, (C4) amphoteric surfactants and (C5) semi-polar surfactants.

As examples of the aforementioned (C1) anionic surfactants, mention may be made of saturated or unsaturated fatty acid salts such as sodium laurate, sodium stearate, sodium oleate, sodium linoleate and the like; alkylsulfuric acid salts; alkylbenzenesulfonic acids such as hexylbenzenesulfonic acid, octylbenzenesulfonic acid, dodecylbenzenesulfonic acid and the like, as well as salts thereof; polyoxyalkylene alkyl ether sulfuric acid salts; polyoxyalkylene alkenyl ether sulfuric acid salts; polyoxyethylene alkylsulfuric ester salts; sulfosuccinic acid alkyl ester salts; polyoxyalkylene sulfosuccinic acid alkyl ester salts; polyoxyalkylene alkylphenyl ether sulfuric acid salts; alkanesulfonic acid salts; octyltrimethylammonium hydroxide; dodecyltrimethylammonium hydroxide; alkyl sulfonates; polyoxyethylene alkylphenyl ether sulfuric acid salts; polyoxyalkylene alkyl ether acetic acid salts; alkyl phosphoric acid salts; polyoxyalkylene alkyl ether phosphoric acid salts; acylglutamic acid salts; α-acylsulfonic acid salts; alkylsulfonic acid salts; alkylallylsulfonic acid salts; α-olefinsulfonic acid salts; alkylnaphthalene sulfonic acid salts; alkanesulfonic acid salts; alkyl- or alkenylsulfuric acid salts; alkylamidesulfuric acid salts; alkyl- or alkenylphosphoric acid salts; alkylamidephosphoric acid salts; alkyloylalkyl taurine salts; N-acylamino acid salts; sulfosuccinic acid salts; alkyl ether carboxylic acid salts; amide ether carboxylic acid salts; α-sulfofatty acid ester salts; alanine derivatives; glycine derivatives; and arginine derivatives. As examples of salts, mention may be made of alkali metal salts such as a sodium salt and the like, alkaline earth metal salts such as a magnesium salt and the like, alkanolamine salts such as a triethanolamine salt and the like, and an ammonium salt.

As examples of the aforementioned (C2) cationic surfactants, mention may be made of alkyltrimethylammonium chloride, stearyltrimethylammonium chloride, lauryltrimethylammonium chloride, cetyltrimethylammonium chloride, beef tallow alkyltrimethylammonium chloride, behenyltrimethylammonium chloride, stearyltrimethylammonium bromide, behenyltrimethylammonium bromide, distearyldimethylammonium chloride, dicocoyldimethylammonium chloride, dioctyldimethylammonium chloride, di(POE) oleylmethylammonium (2 EO) chloride, benzalkonium chloride, alkyl benzalkonium chloride, alkyl dimethylbenzalkonium chloride, benzethonium chloride, stearyl dimethylbenzylammonium chloride, lanolin derivative quaternary ammonium salt, stearic acid diethylaminoethylamide, stearic dimethylaminopropylamide, behenic acid amide propyldimethyl hydroxypropylammonium chloride, stearoyl colaminoformyl methylpyridinium chloride, cetylpyridinium chloride, tall oil alkylbenzyl hydroxyethylimidazolinium chloride, and benzylammonium salt.

As examples of the aforementioned (C3) nonionic surfactants, mention may be made of polyoxyalkylene ethers, polyoxyalkylene alkyl ethers, polyoxyalkylene fatty acid esters, polyoxyalkylene fatty acid diesters, polyoxyalkylene resin acid esters, polyoxyalkylene (hardened) castor oils, polyoxyalkylene alkyl phenols, polyoxyalkylene alkyl phenyl ethers, polyoxyalkylene phenyl phenyl ethers, polyoxyalkylene alkyl esters, polyoxyalkylene alkyl esters, sorbitan fatty acid esters, polyoxyalkylene sorbitan alkyl esters, polyoxyalkylene sorbitan fatty acid esters, polyoxyalkylene sorbitol fatty acid esters, polyoxyalkylene glycerol fatty acid esters, polyglycerol alkyl ethers, polyglycerol fatty acid esters, sucrose fatty acid esters, fatty acid alkanolamides, alkylglucosides, polyoxyalkylene fatty acid bisphenyl ethers, polypropylene glycol, diethylene glycol, polyoxyalkylene-modified silicones, polyglyceryl-modified silicones, glyceryl-modified silicones, sugar-modified silicones, fluorine-based surfactants, polyoxyethylene/polyoxypropylene block polymers, and alkyl polyoxyethylene/polyoxypropylene block polymer ethers. A polyoxyalkylene-modified silicone, a polyglycerol-modified silicone, or a glycerol-modified silicone in which an alkyl branch, a linear silicone branch, a siloxane dendrimer branch or the like may be possessed together with a hydrophilic group at the same time, if necessary, can also be preferably used.

The organo-modified silicone already described as the aforementioned (B) oil agent may possess an aspect as a nonionic emulsifier depending on the structure thereof, in addition to an aspect as an oil agent. Namely, the organo-modified silicone oils such as a polyoxyalkylene-modified silicone, a polyglycerol-modified silicone, a glycerol-modified silicone and the like, possessing both a hydrophilic moiety and a hydrophobic moiety in a molecule possess a function as a nonionic surfactant. In addition, the aforementioned (A) sugar alcohol-modified silicone, per se, possesses the aforementioned function. They may function as an auxiliary agent for improving stability of the aforementioned (C3) nonionic surfactant and may improve stability of the entire preparation. Therefore, they can be used in combination.

As examples of the aforementioned (C4) amphoteric surfactants, mention may be made of imidazoline-type, amidobetaine-type, alkylbetaine-type, alkylamidobetaine-type, alkylsulfobetaine-type, amidosulfobetaine-type, hydroxysulfobetaine-type, carbobetaine-type, phosphobetaine-type, aminocarboxylic acid-type, and amidoamino acid-type amphoteric surfactants. More particularly, as examples thereof, mention may be made of imidazoline-type amphoteric surfactants such as sodium 2-undecyl-N,N,N-(hydroxyethylcarboxymethyl)-2-imidazoline, 2-cocoyl-2-imidazolinium hydroxide-1-carboxyethyloxy disodium salt and the like; alkylbetaine-type amphoteric surfactants such as lauryl dimethylaminoacetic acid betaine, myristyl betaine and the like; and amidobetaine-type amphoteric surfactants such as coconut oil fatty acid amidopropyl dimethylamino acetic acid betaine, palm kernel oil fatty acid amidopropyl dimethylamino acetic acid betaine, beef tallow fatty acid amidopropyl dimethylamino acetic acid betaine, hardened beef tallow fatty acid amidopropyl dimethylamino acetic acid betaine, lauric amidopropyl dimethylamino acetic acid betaine, myristic amidopropyl dimethylamino acetic acid betaine, palmitic amidopropyl dimethylamino acetic acid betaine, stearic amidopropyl dimethylamino acetic acid betaine, oleic amidopropyl dimethylamino acetic acid betaine and the like; alkyl sulfobetaine-type amphoteric surfactants such as coconut oil fatty acid dimethyl sulfopropyl betaine and the like; alkylhydroxy sulfobetaine-type amphoteric surfactants such as lauryl dimethylaminohydroxy sulfobetaine and the like; phosphobetaine-type amphoteric surfactants such as laurylhydroxy phosphobetaine and the like; amidoamino acid-type amphoteric surfactants such as sodium N-lauroyl-N′-hydroxyethyl-N′-carboxymethyl ethylenediamine, sodium N-oleoyl-N′-hydroxyethyl-N′-carboxymethyl ethylenediamine, sodium N-cocoyl-N′-hydroxyethyl-N′-carboxymethyl ethylenediamine, potassium N-lauroyl-N′-hydroxyethyl-N′-carboxymethyl ethylenediamine, potassium N-oleoyl-N′-hydroxyethyl-N′-carboxymethyl ethylenediamine, sodium N-lauroyl-N-hydroxyethyl-N′-carboxymethyl ethylenediamine, sodium N-oleoyl-N-hydroxyethyl-N′-carboxymethyl ethylenediamine, sodium N-cocoyl-N-hydroxyethyl-N′-carboxymethyl ethylenediamine, monosodium N-lauroyl-N-hydroxyethyl-N′,N′-dicarboxymethyl ethylenediamine, monosodium N-oleoyl-N-hydroxyethyl-N′,N′-dicarboxymethyl ethylenediamine, monosodium N-cocoyl-N-hydroxyethyl-N′,N′-dicarboxymethyl ethylenediamine, disodium N-lauroyl-N-hydroxyethyl-N′,N′-dicarboxymethyl ethylenediamine, disodium N-oleoyl-N-hydroxyethyl-N′,N′-dicarboxymethyl ethylenediamine, disodium N-cocoyl-N-hydroxyethyl-N′,N′-dicarboxymethyl ethylenediamine and the like.

As examples of the aforementioned (C5) semi-polar surfactants, mention may be made of alkylamine oxide-type surfactants, alkylamine oxides, alkylamide amine oxides, alkylhydroxyamine oxides and the like. Alkyldimethylamine oxides having 10 to 18 carbon atoms, alkoxyethyl dihydroxyethylamine oxides having 8 to 18 carbon atoms and the like are preferably used. More particularly, as examples thereof, mention may be made of dodecyldimethylamine oxide, dimethyloctylamine oxide, diethyldecylamine oxide, bis-(2-hydroxyethyl) dodecylamine oxide, dipropyltetradecylamine oxide, methylethylhexadecylamine oxide, dodecylamidopropyl dimethylamine oxide, cetyldimethylamine oxide, stearyldimethylamine oxide, tallow dimethylamine oxide, dimethyl-2-hydroxyoctadecylamine oxide, lauryldimethylamine oxide, myristyldimethylamine oxide, stearyldimethylamine oxide, isostearyldimethylamine oxide, coconut fatty acid alkyldimethylamine oxide, caprylic amide propyldimethylamine oxide, capric amide propyldimethylamine oxide, lauric amide propyldimethylamine oxide, myristic amide propyldimethylamine oxide, palmitic amide propyldimethylamine oxide, stearic amide propyldimethylamine oxide, isostearic amide propyldimethylamine oxide, oleic amide propyldimethylamine oxide, ricinoleic amide propyldimethylamine oxide, 12-hydroxystearic amide propyldimethylamine oxide, coconut fatty acid amide propyldimethylamine oxide, palm kernel oil fatty acid amide propyldimethylamine oxide, castor oil fatty acid amide propyldimethylamine oxide, lauric amide ethyldimethylamine oxide, myristic amide ethyldimethylamine oxide, coconut fatty acid amide ethyldimethylamine oxide, lauric amide ethyldiethylamine oxide, myristic amide ethyldiethylamine oxide, coconut fatty acid amide ethyldiethylamine oxide, lauric amide ethyldihydroxyethylamine oxide, myristic amide ethyldihydroxyethylamine oxide, and coconut fatty acid amide ethyldihydroxyethylamine oxide.

The blending amount of the aforementioned (C) surfactants in the cosmetic for hair of the present invention is not particularly restricted. In order to improve a cleansing property, the surfactants can be blended in an amount ranging from 0.1 to 90% by weight (mass) and preferably ranging from 1 to 50% by weight (mass) in the total amount of the cosmetic composition. In view of a cleansing property, the amount is preferably 25% by weight (mass) or more.

Water-Soluble Polymers

The cosmetic for hair of the present invention preferably comprises (D) a water-soluble polymer. The aforementioned (D) water-soluble polymer may be blended in order to prepare a cosmetic for hair in the desirable form, and improve a sensation during use of the cosmetic for hair such as a feeling on touch with respect to hair or the like, a conditioning effect or the like.

As the aforementioned (D) water-soluble polymer, any one of amphoteric, cationic, anionic, nonionic, and water-swellable clay minerals can be used as long as they are commonly used in a cosmetic for hair. One type or two or more types of water-soluble polymers can be used. The aforementioned (D) water-soluble polymers have an effect of thickening a hydrous component, and for this reason, they are useful in the case of obtaining a hydrous cosmetic for hair, and in particular, in the form of a gel hydrous cosmetic for hair, a water-in-oil emulsion cosmetic for hair, and an oil-in-water emulsion cosmetic for hair.

As examples of natural water-soluble polymers, mention may be made of vegetable-based polymers such as gum Arabic, tragacanth gum, galactan, guar gum, carob gum, karaya gum, carrageenan, pectin, agar, quince seed, algal colloid, starch (rice, corn, potato, or wheat), glycyrrhizinic acid and the like; microorganism-based polymers such as xanthan gum, dextran, succinoglucan, pullulan, and the like; and animal-based polymers such as collagen, casein, albumin, gelatin, and the like. In addition, as examples of semi-synthetic water-soluble polymers, mention may be made of, for example, starch-based polymers such as carboxymethyl starch, methylhydroxypropyl starch, and the like; cellulose-based polymers such as methylcellulose, nitrocellulose, ethylcellulose, methylhydroxypropylcellulose, hydroxyethylcellulose, sodium cellulose sulfate, hydroxypropylcellulose, sodium carboxymethylcellulose (CMC), crystalline cellulose, cellulose powder, and the like; and alginate-based polymers such as sodium alginate, propylene glycol alginate and the like. As examples of synthetic water-soluble polymers, mention may be made of, for example, vinyl-based polymers such as polyvinyl alcohol, polyvinyl methyl ether-based polymer, polyvinylpyrrolidone, carboxyvinyl polymer (CARBOPOL 940, CARBOPOL 941; manufactured by The Lubrizol Corporation); polyoxyethylene-based polymers such as polyethylene glycol 20,000, polyethylene glycol 6,000, polyethylene glycol 4,000 and the like; copolymer-based polymers such as a copolymer of polyoxyethylene and polyoxypropylene, PEG/PPG methyl ether and the like; acryl-based polymers such as poly(sodium acrylate), poly(ethyl acrylate), polyacrylamide and the like; polyethylene imines; cationic polymers and the like. The water-swellable clay minerals are nonionic water-soluble polymers and correspond to one type of colloid-containing aluminum silicate having a triple layer structure. More particular, as examples thereof, mention may be made of bentonite, montmorillonite, beidellite, nontronite, saponite, hectorite, aluminum magnesium silicate, and silicic anhydride. They may be any one of natural ones and synthesized ones.

As examples of components which can be preferably blended in a cosmetic for hair, mention may be made of, in particular, (D1) cationic water-soluble polymers. As examples of the aforementioned (D1) cationic water-soluble polymers, mention may be made of quaternary nitrogen-modified polysaccharides such as cation-modified cellulose, cation-modified hydroxyethylcellulose, cation-modified guar gum, cation-modified locust bean gum, cation-modified starch and the like; dimethyldiallylammonium chloride derivatives such as a copolymer of dimethyldiallylammonium chloride and acrylamide, poly(dimethylmethylene piperidinium chloride) and the like; vinylpyrrolidone derivatives such as a salt of a copolymer of vinylpyrrolidone and dimethylaminoethyl methacrylic acid, a copolymer of vinylpyrrolidone and methacrylamide propyltrimethylammonium chloride, a copolymer of vinylpyrrolidone and methylvinylimidazolium chloride and the like; and methacrylic acid derivatives such as a copolymer of methacryloylethyldimethylbetaine, methacryloylethyl trimethylammonium chloride and 2-hydroxyethyl methacrylate, a copolymer of methacryloylethyldimethylbetaine, methacryloylethyl trimethylammonium chloride and methoxy polyethylene glycol methacrylate, and the like.

In addition, in particular, as a component which can be preferably blended in a cosmetic for hair, (D2) an amphoteric water-soluble polymer can be mentioned. More particularly, as examples thereof, mention may be made of amphoterized starches; dimethyldiallylammonium chloride derivatives such as a copolymer of acrylamide, acrylic acid, and dimethyldiallylammonium chloride, and a copolymer of acrylic acid and dimethyldiallylammonium chloride; and methacrylic acid derivatives such as polymethacryloylethyl dimethylbetaine, a copolymer of methacryloyloxyethyl carboxybetaine and alkyl methacrylate, a copolymer of octylacrylamide, hydroxypropyl acrylate and butylaminoethyl methacrylate, and a copolymer of N-methacryloyloxyethyl N,N-dimethylammonium α-methylcarboxybetaine and alkyl methacrylate.

The blending amount of the aforementioned (D) water-soluble polymer in the cosmetic for hair of the present invention can be suitably selected in accordance with the type and purpose of the cosmetic for hair. The amount may preferably range from 0.01 to 5.0% by weight (mass) and more preferably range from 0.1 to 3.0% by weight (mass) with respect to the total amount of the cosmetic for hair in order to particularly obtain a superior sensation during use. If the blending amount of the water-soluble polymer exceeds the aforementioned upper limit, a rough feeling with respect to the hair may remain in some types of the cosmetics for hair. On the other hand, if the blending amount is below the aforementioned lower limit, advantageous technical effects such as a thickening effect, a conditioning effect and the like may not be sufficiently exhibited.

Alcohols

The cosmetic for hair of the present invention preferably comprises (E) an alcohol. As the aforementioned (E) alcohols, one or more types of polyhydric alcohols and/or a monovalent lower alcohols can be used. As examples of lower alcohols, mention may be made of ethanol, isopropanol, n-propanol, t-butanol, s-butanol and the like. As examples of polyhydric alcohols, mention may be made of divalent alcohols such as 1,3-propanediol, 1,3-butylene glycol, 1,2-butylene glycol, propylene glycol, trimethylene glycol, tetramethylene glycol, 2,3-butylene glycol, pentamethylene glycol, 2-buten-1,4-diol, dibutylene glycol, pentyl glycol, hexylene glycol, octylene glycol and the like; trivalent alcohols such as glycerol, trimethylol propane, 1,2,6-hexanetriol and the like; polyhydric alcohols having 4 or more valences such as pentaerythritol, xylitol and the like; and sugar alcohols such as sorbitol, mannitol, maltitol, maltotriose, sucrose, erythritol, glucose, fructose, a starch-decomposed product, maltose, xylitose, starch-decomposed sugar-reduced alcohol and the like. In addition to the aforementioned low-molecule polyhydric alcohols, polyhydric alcohol polymers such as diethylene glycol, dipropylene glycol, triethylene glycol, propylene glycol, tetraethylene glycol, diglycerol, polyethylene glycol, triglycerol, tetraglycerol, polyglycerol and the like may be mentioned. Among these, 1,3-propanediol, 1,3-butylene glycol, sorbitol, dipropylene glycol, glycerol, and polyethylene glycol are, in particular, preferred.

The blending amount of the aforementioned (E) alcohols preferably ranges from 0.1 to 50% by weight (mass) with respect to the total amount of the cosmetic for hair. Alcohols can be blended in an amount ranging from about 5 to 30% by weight (mass) with respect to the total amount of the cosmetic for hair in order to improve storage stability of the cosmetic for hair. This is one preferable mode for carrying out the present invention.

Thickening Agents and/or Gelling Agents

The cosmetic for hair of the present invention preferably further comprises (F) a thickening agent and/or a gelling agent. As an aqueous thickening and/or gelling agent, the aforementioned water-soluble polymers of component (D) described above are preferably used. In addition, as examples of oil-soluble thickening and/or gelling agents, mention may be made of metallic soaps such as aluminum stearate, magnesium stearate, zinc myristate and the like; amino acid derivatives such as N-lauroyl-L-glutamic acid, α,γ-di-n-butylamine and the like; dextrin fatty acid esters such as dextrin palmitate, dextrin stearate, dextrin 2-ethylhexanoate palmitate and the like; sucrose fatty acid esters such as sucrose palmitate, sucrose stearate and the like; benzylidene derivatives of sorbitol such as monobenzylidene sorbitol, dibenzylidene sorbitol and the like; and the like. The thickening and/or gelling agents can be used alone or in combination of two or more types thereof, if necessary.

As the aforementioned (F) thickening and/or gelling agent, an organo-modified clay mineral can be used. The organo-modified clay mineral can be used as a gelling agent for the oil agent(s) in the same manner as described in the aforementioned oil-soluble thickening and/or gelling agent. As examples of organo-modified clay minerals, mention may be made of, for example, dimethylbenzyl dodecylammonium montmorillonite clay, dimethyldioctadecylammonium montmorillonite clay, dimethylalkylammonium hectorite, benzyldimethylstearylammonium hectorite, distearyldimethylammonium chloride-treated aluminum magnesium silicate and the like. As examples of commercially available products thereof, mention may be made of Benton 27 (benzyldimethylstearylammonium chloride-treated hectorite, manufactured by Nationalred Co.), Benton 38 (distearyldimethylammonium chloride-treated hectorite, manufactured by Nationalred Co.) and the like.

The usage amount of the aforementioned (F) thickening and/or gelling agent in the cosmetic for hair of the present invention is not particularly restricted, and may preferably range from 0.5 to 50 parts by weight (mass), and more preferably range from 1 to 30 parts by weight (mass), with respect to 100 parts by weight (mass) of the oil agent(s). The ratio thereof in the cosmetic for hair preferably ranges from 0.01 to 30% by weight (mass), more preferably ranges from 0.1 to 20% by weight (mass), and furthermore preferably ranges from 1 to 10% by weight (mass)

By thickening or gelling the oil agent(s) in the cosmetic for hair of the present invention, the viscosity or hardness of the cosmetic can be made appropriate, and the outer appearance, blending properties, and the sensation during use can be improved. In addition, a desirable formulation and/or a desirable form of the cosmetic can be achieved. When the other (F) thickening and/or gelling agent is used, in addition thereto, there are advantages in view of qualities in that an oily sensation (oily and sticky feeling on touch) can be further totally controlled, and a hair-retaining property can be further improved.

Powder

The cosmetic for hair of the present invention can further comprise (G) powder. “Powder” in the present invention is that commonly used as a component of a cosmetic, and includes white and colored pigments and extender pigments. The white and colored pigments are used in coloring a cosmetic, and on the other hand, the extender pigments are used in improvement in a feeling on touch of a cosmetic and the like. As the aforementioned (G) powder in the present invention, white or colored pigments and extender pigments which are commonly used in cosmetics can be used without any restrictions. One type of powder may be used, or two or more types of powders may be preferably blended.

With respect to the aforementioned (G) powders, there is no restriction on the form thereof (sphere, bar, needle, plate, amorphous, spindle or the like), the particle size (aerosol, microparticle, pigment-grade particle, or the like), and the particle structure (porous, non-porous or the like) thereof. The average primary particle size of the powders preferably ranges from 1 nm to 100 μm.

As examples of the aforementioned (G) powders, mention may be made of, for example, inorganic powders, organic powders, surfactant metal salt powders (metallic soaps), colored pigments, pearl pigments, metal powder pigments and the like. In addition, hybrid products of the aforementioned pigments can also be used.

More particularly, as examples of inorganic powders, mention may be made of titanium oxide, zirconium oxide, zinc oxide, cerium oxide, magnesium oxide, barium sulfate, calcium sulfate, magnesium sulfate, calcium carbonate, magnesium carbonate, talc, mica, kaolin, sericite, white mica, synthetic mica, phlogopite, lepidolite, black mica, lithia mica, silicic acid, silicic acid anhydride, aluminum silicate, sodium silicate, magnesium sodium silicate, magnesium silicate, aluminum magnesium silicate, calcium silicate, barium silicate, strontium silicate, metal salts of tungstic acid, hydroxyapatite, vermiculite, higilite, bentonite, montmorillonite, hectorite, zeolite, ceramic powder, dicalcium phosphate, alumina, aluminum hydroxide, boron nitride, and the like.

As examples of organic powders, mention may be made of polyamide powder, polyester powder, polyethylene powder, polypropylene powder, polystyrene powder, polyurethane powder, benzoguanamine powder, polymethylbenzoguanamine powder, polytetrafluoroethylene powder, poly(methyl methacrylate) powder, cellulose, silk powder, nylon powder, nylon 12, nylon 6, silicone powder, polymethylsilsesquioxane spherical powder, copolymers of styrene and acrylic acid, copolymers of divinylbenzene and styrene, vinyl resin, urea resin, phenol resin, fluorine resin, silicone resin, acrylic resin, melamine resin, epoxy resin, polycarbonate resin, microcrystalline fiber powder, starch powder, lauroyl lysine and the like.

As examples of surfactant metal salt powders, mention may be made of zinc stearate, aluminum stearate, calcium stearate, magnesium stearate, zinc myristate, magnesium myristate, zinc palmitate, zinc laurate, zinc cetylphosphate, calcium cetylphosphate, sodium zinc cetylphosphate, and the like.

As examples of colored pigments, mention may be made of inorganic red pigments such as red iron oxide, iron oxide, iron hydroxide, iron titanate and the like; inorganic brown pigments such as gamma-iron oxide and the like; inorganic yellow pigments such as yellow iron oxide, ocher, and the like; inorganic black iron pigments such as black iron oxide, carbon black and the like; inorganic purple pigments such as manganese violet, cobalt violet, and the like; inorganic green pigments such as chromium hydroxide, chromium oxide, cobalt oxide, cobalt titanate, and the like; inorganic blue pigments such as Prussian blue, ultramarine blue, and the like; laked pigments of tar pigments such as Red No. 3, Red No. 104, Red No. 106, Red No. 201, Red No. 202, Red No. 204, Red No. 205, Red No. 220, Red No. 226, Red No. 227, Red No. 228, Red No. 230, Red No. 401, Red No. 505, Yellow No. 4, Yellow No. 5, Yellow No. 202, Yellow No. 203, Yellow No. 204, Yellow No. 401, Blue No. 1, Blue No. 2, Blue No. 201, Blue No. 404, Green No. 3, Green No. 201, Green No. 204, Green No. 205, Orange No. 201, Orange No. 203, Orange No. 204, Orange No. 206, Orange No. 207 and the like, laked pigments of natural pigments such as carminic acid, laccaic acid, carthamin, brazilin, crocin and the like.

As examples of pearl pigments, mention may be made of titanium oxide-coated mica, titanium mica, iron oxide-coated titanium mica, titanium oxide-coated mica, bismuth oxychloride, titanium oxide-coated bismuth oxychloride, titanium oxide-coated talc, fish scale foil, titanium oxide-coated colored mica, and the like.

As examples of metal powder pigments, mention may be made of powders of metals such as aluminum, gold, silver, copper, platinum, stainless steel, and the like.

In addition, in the aforementioned (G) powders, a part or all parts thereof may, in particular, preferably be subjected to a surface treatment such as a water-repellent treatment, a hydrophilic treatment or the like. In addition, composited products in which the aforementioned powders are mutually composited may be used. In addition, surface-treated products in which the aforementioned powders have been subjected to a surface treatment with a general oil agent, a silicone compound other than the aforementioned (A) sugar alcohol-modified silicone of the present invention, a fluorine compound, a surfactant, a thickening agent or the like can also be used. One type thereof or two or more types thereof can be used, as necessary.

The water-repellant treatments are not particularly restricted. The aforementioned (G) powders can be treated with various types of water-repellant surface treatment agents. As examples thereof, mention may be made of organosiloxane treatments such as a methylhydrogenpolysiloxane treatment, a silicone resin treatment, a silicone gum treatment, an acryl silicone treatment, a fluorinated silicone treatment and the like; metallic soap treatments such as a zinc stearate treatment and the like; silane treatments such as a silane coupling agent treatment, an alkylsilane treatment and the like; fluorine compound treatments such as a perfluoroalkylsilane treatment, a perfluoroalkyl phosphate treatment, a perfluoro polyether treatment and the like; amino acid treatments such as an N-lauroyl-L-lysine treatment and the like; oil agent treatments such as a squalane treatment and the like; acryl treatments such as an alkyl acrylate treatment and the like. The aforementioned treatments can be used in combination of two or more types thereof.

As the aforementioned (G) powders, silicone elastomer powders can also be used. The silicone elastomer powder is a crosslinked product of a linear diorganopolysiloxane mainly formed from a diorganosiloxane unit (D unit). The silicone elastomer powder can be preferably produced by crosslink-reacting an organohydrogenpolysiloxane having a silicon-binding hydrogen atom at the side chain or the terminal and a diorganopolysiloxane having an unsaturated hydrocarbon group such as an alkenyl group or the like at the side chain or the terminal, in the presence of a catalyst for a hydrosilylation reaction. The silicone elastomer powder has. an increased flexibility and elasticity, and exhibits a superior oil-absorbing property, as compared with a silicone resin powder formed from T units and Q units. For this reason, the silicone elastomer powder absorbs sebum on the skin and can prevent makeup running.

The silicone elastomer powders can be in various forms such as a spherical form, a flat form, an amorphous form and the like. The silicone elastomer powders may be in the form of an oil dispersant. In the cosmetic of the present invention, silicone elastomer powders in the form of particles, which have a primary particle size observed by an electron microscope and/or an average primary particle size measured by a laser diffraction/scattering method ranging from 0.1 to 50 μm, and in which the primary particle is in a spherical form, can be preferably blended. In addition, the silicone elastomer constituting the silicone elastomer powders may have a hardness preferably not exceeding 80, and more preferably not exceeding 65, when measured by means of a type A durometer according to JIS K 6253 “Method for determining hardness of vulcanized rubber or thermoplastic rubber”.

The aforementioned silicone elastomer powders can be used in the cosmetic for hair of the present invention, in the form of an aqueous dispersion. As examples of commercially available products of the aforementioned aqueous dispersions, mention may be made of, for example, “BY 29-129” and “PF-2001 PIF Emulsion” manufactured by Dow Corning Toray Co., Ltd., and the like. By blending an aqueous dispersion (=suspension) of the aforementioned silicone elastomer powders, a sensation during use of the cosmetics for hair, and in particular, the cosmetics for hair in the form of an oil-in-water emulsion can be further improved.

The silicone elastomer powders may be subjected to a surface treatment with a silicone resin, silica or the like. As examples of the aforementioned surface treatments, mention may be made of, for example, those described in Japanese Unexamined Patent Application, First Publication No. H02-243612; Japanese Unexamined Patent Application, First Publication No. H08-12545; Japanese Unexamined Patent Application, First Publication No. H08-12546; Japanese Unexamined Patent Application, First Publication No. H08-12524; Japanese Unexamined Patent Application, First Publication No. H09-241511; Japanese Unexamined Patent Application, First Publication No. H10-36219; Japanese Unexamined Patent Application, First Publication No. H11-193331; Japanese Unexamined Patent Application, First Publication No. 2000-281523 and the like. As the silicone elastomer powders, crosslinking silicone powders listed in “Japanese Cosmetic Ingredients Codex (JCIC)” correspond thereto. As commercially available products, there are Trefil E-506S, Trefil E-508, 9701 Cosmetic Powder, and 9702 Powder, manufactured by Dow Corning Toray Co., Ltd., and the like. As examples of the surface treatment agents, mention may be made of methylhydrogenpolysiloxane, silicone resins, metallic soap, silane coupling agents, inorganic oxides such as silica, titanium oxide and the like and fluorine compounds such as perfluoroalkylsilane, perfluoroalkyl phosphoric ester salts and the like.

The blending amount of the aforementioned (G) powder in the cosmetic for hair of the present invention is not particularly restricted, and may preferably range from 0.1 to 50% by weight (mass), more preferably range from 1 to 30% by weight (mass), and furthermore preferably range from 5 to 15% by weight (mass) with respect to the total amount of the cosmetic.

Solid Silicone Resin or Crosslinking organopolysiloxane

The cosmetic for hair of the present invention can further comprise (H) a solid silicone resin or crosslinking organopolysiloxane. The solid silicone resin or crosslinking organopolysiloxane is preferably hydrophobic so that it is completely insoluble in water at room temperature or the solubility thereof with respect to 100 g of water is below 1% by weight (mass).

The aforementioned (H) solid silicone resin or crosslinking organopolysiloxane is an organopolysiloxane with a highly branched molecular structure, a net-like molecular structure or a cage-like molecular structure, and may be in the form of a liquid or solid at room temperature. Any silicone resins usually used in cosmetics for hair can be used unless they are contrary to the purposes of the present invention. In the case of a solid silicone resin, the silicone resin may be in the form of particles such as spherical powders, scale powders, needle powders platy flake powders (including platy powders having an aspect ratio of particles and the outer appearance which are generally understood as a plate form) or the like. In particular, silicone resin powders containing a monoorganosiloxy unit (T unit) and/or a siloxy unit (Q unit) described below are preferably used.

Blending the aforementioned (H) solid silicone resin together with the aforementioned (A) sugar alcohol-modified silicone is useful, since the miscibility with the aforementioned (B) oil agents and the uniformly dispersing property can be improved, and at the same time, an effect of improving a sensation during use such as uniform adhesiveness with respect to the part to be applied, obtained in accordance with blending the aforementioned (H) solid silicone resin can be obtained.

As examples of the aforementioned (H) solid silicone resins, mention may be made of, for example, MQ resins, MDQ resins, MTQ resins, MDTQ resins, TD resins, TQ resins, or TDQ resins comprising any combinations of a triorganosiloxy unit (M unit) (wherein the organo group is a methyl group alone, or a methyl group in combination with a vinyl group or a phenyl group), a diorganosiloxy unit (D unit) (wherein the organo group is a methyl group alone, or a methyl group in combination with a vinyl group or a phenyl group), a monoorganosiloxy unit (T unit) (wherein the organo group is a methyl group, a vinyl group or a phenyl group), and a siloxy unit (Q unit). In addition, as other examples thereof, mention may be made of trimethylsiloxysilicic acid, polyalkylsiloxysilicic acid, trimethylsiloxysilicic acid containing dimethylsiloxy units and alkyl(perfluoroalkyl) siloxysilicic acid. The aforementioned silicone resins are preferably oil soluble, and, in particular, preferably are soluble in a volatile silicone.

In particular, a phenyl silicone resin with an increased refractive index which has an increased content of a phenyl group (such as 217 Flake Resin manufactured by Dow Corning Toray Co., Ltd.) can easily form silicone resin powders in the form of flakes. In the case of blending the powders in a cosmetic for hair, a brilliant transparent impression can be provided to the skin and hair.

The aforementioned (H) crosslinking organopolysiloxane preferably has a structure in which an organopolysiloxane chain is three-dimensionally crosslinked by a reaction with a crosslinking component formed from a polyether unit, an alkylene unit having 4 to 20 carbon atoms, and an organopolysiloxane unit, or the like.

The aforementioned (H) crosslinking organopolysiloxane can be particularly obtained by addition-reacting an organohydrogenpolysiloxane having silicon-binding hydrogen atoms, a polyether compound having unsaturated bonds at both terminals of the molecular chain, an unsaturated hydrocarbon having more than one double bonds in a molecule, and an organopolysiloxane having more than one double bonds in a molecule. Here, the crosslinking organopolysiloxane may or may not have a modifying functional group such as an unreacted silicon-binding hydrogen atom, an aromatic hydrocarbon group such as a phenyl group or the like, a long chain alkyl group having 6 to 30 carbon atoms such as an octyl group, a polyether group, a carboxyl group, a silylalkyl group having the aforementioned carbosiloxane dendrimer structure or the like, and can be used without restrictions of physical modes and preparation methods such as dilution, properties and the like.

As one example, the aforementioned crosslinking organopolysiloxane can be obtained by addition-reacting an organohydrogenpolysiloxane which is formed from a structure unit selected from the group consisting of a SiO₂ unit, a HSiO_{1.5} unit, a R^{b}SiO_{1.5} unit, a R^{b}HSiO unit, a R^{b}₂SiO unit, a R^{b}₃SiO_{0.5} unit and a R^{b}₂HSiO_{0.5} unit, wherein R^{b} is a substituted or non-substituted monovalent hydrocarbon group having 1 to 30 carbon atoms, excluding an aliphatic unsaturated group, and a part of R^{b} is a monovalent hydrocarbon group having 8 to 30 carbon atoms, and at the same time, includes 1.5 or more, on average, of hydrogen atoms binding to the silicon atom in the molecule, with a crosslinking component selected from the group consisting of a polyoxyalkylene compound having unsaturated hydrocarbon groups at both terminals of the molecular chain, a polyether compound such as a polyglycerol compound, a polyglycidyl ether compound or the like, an unsaturated hydrocarbon which is an α,ω-diene represented by the following general formula: CH₂═CH—CᵣH₂ᵣ—CH═CH₂, wherein r is an integer ranging from 0 to 26, and an organopolysiloxane which is formed from a SiO₂ unit, a (CH₂═CH)SiO_{1.5} unit, a R^{c}SiO_{1.5} unit, a R^{c}(CH₂═CH)SiO unit, a R^{c}₂SiO unit, a R^{c}₃SiO_{0.5}, and a R^{c}₂(CH₂═CH)SiO_{0.5}, wherein R^{c} is a substituted or non-substituted monovalent hydrocarbon group having 1 to 30 carbon atoms, excluding an aliphatic unsaturated group, and includes 1.5 or more, on average, of vinyl groups binding to the silicon atom. The aforementioned modifying functional group can be introduced by carrying out an addition reaction with respect to the unreacted hydrogen atoms binding to the silicon atom in a molecule. For example, 1-hexene is reacted with a crosslinking organopolysiloxane having an unreacted hydrogen atom binding to the silicon atom, and thereby, a hexyl group which is an alkyl group having 6 carbon atoms can be introduced thereinto.

The aforementioned crosslinking organopolysiloxanes can be used without restrictions of physical modes and preparation methods such as dilution, properties and the like. As particularly preferable examples thereof, mention may be made of α,ω-diene crosslinking silicone elastomers (as commercially available products, DC 9040 Silicone Elastomer Blend, DC 9041 Silicone Elastomer Blend, DC 9045 Silicone Elastomer Blend, and DC 9046 Silicone Elastomer Blend, manufactured by Dow Corning Corporation in the USA) described in U.S. Pat. No. 5,654,362. In the same manner as described above, as examples of partially crosslinking organopolysiloxane polymers, mention may be made of (dimethicone/vinyldimethicone) crosspolymer, (dimethicone/phenylvinyldimethicone) crosspolymer, (PEG-8 to 30/C6 to C30 alkyldimethicone) crosspolymer, (vinyldimethicone/C6 to C30 alkyldimethicone) crosspolymer, (dimethicone/polyglycerol) crosspolymer and the like, in the case of using INCI names (International Nomenclature Cosmetic Ingredient labeling names).

In the case of blending an emulsifiable crosslinking organopolysiloxane formed by crosslinking by means of a polyether compound in a cosmetic for hair as a component, the aforementioned (A) sugar alcohol-modified silicone can function as a dispersant. For this reason, there is an advantage in that a uniform emulsification system can be formed.

On the other hand, in the case of blending a non-emulsifiable crosslinking organopolysiloxane formed by crosslinking by means of an unsaturated hydrocarbon group such as a diene or an organopolysiloxane in a cosmetic for hair as a component, an adhesive sensation to the hair can be improved. In addition, there is an advantage in that good compatibility with other oil agents can be exhibited, and the whole oil system can be uniformly and stably blended in the cosmetic for hair.

The aforementioned (H) solid silicone resin or crosslinking organopolysiloxane can be blended alone or in combination with two or more types thereof in accordance with the purpose thereof. The solid silicone resin or crosslinking organopolysiloxane may be blended in an amount preferably ranging from 0.05 to 25% by weight (mass) and more preferably ranging from 0.1 to 15% by weight (mass), with respect to the total amount of the cosmetic for hair, in accordance with the purpose and blending intention.

Acryl Silicone Dendrimer Copolymer

The cosmetic for hair of the present invention can further comprise (I) an acryl silicone dendrimer copolymer. The aforementioned (I) acryl silicone dendrimer copolymer is a vinyl-based polymer having a carbosiloxane dendrimer structure at the side chain. As examples thereof, mention may be, in particular, preferably made of vinyl-based polymers described in Japanese Patent No. 4,009,382 (Japanese Unexamined Patent Application, First Publication No. 2000-063225). As examples of commercially available products, mention may be made of FA 4001 CM Silicone Acrylate, and FA 4002 ID Silicone Acrylate, manufactured by Dow Corning Toray Co., Ltd., and the like. An acryl silicone dendrimer copolymer having a long chain alkyl group having 8 to 30 carbon atoms and preferably having 14 to 22 carbon atoms at the side chain or the like may be used. In the case of blending the aforementioned acryl silicone dendrimer copolymer alone, a superior property of forming a film can be exhibited. For this reason, by blending the dendrimer copolymer in the cosmetic for hair according to the present invention, a strong coating film can be formed on the applied part, and durability of a sebum resistance property, a rub resistance property and the like can be considerably improved.

By using the aforementioned (A) sugar alcohol-modified silicone together with the aforementioned (I) acryl silicone dendrimer copolymer, there are advantages in that a surface protective property such as a sebum resistance property can be improved due to strong water repellency provided by the carbosiloxane dendrimer structure, and at the same time, irregularities such as pores and wrinkles of the skin to be applied can be effectively made inconspicuous. In addition, the aforementioned (A) sugar alcohol-modified silicone can provide miscibility of the aforementioned (I) acryl silicone dendrimer copolymer with the other oil agent(s). For this reason, there is an advantage in that degradation of hair can be controlled for a long time.

The blending amount of the aforementioned (I) acryl silicone dendrimer copolymer can appropriately be selected in accordance with the purpose and blending intention. The amount may preferably range from 1 to 99% by weight (mass), and more preferably may range from 30 to 70% by weight (mass), with respect to the total amount of the cosmetic for hair.

UV-Ray Protective Component

The cosmetic for hair of the present invention can further comprise (J) a UV-ray protective component. The aforementioned (J) UV-ray protective component is preferably hydrophobic so that the component is completely insoluble in water at room temperature or the solubility thereof with respect to 100 g of water is below 1% by weight (mass). The aforementioned (J) UV-ray protective component is a component for blocking or diffusing UV rays. Among UV-ray protective components, there are inorganic UV-ray protective components and organic UV-ray protective components. If the cosmetics for hair of the present invention are sunscreen cosmetics, at least one type of inorganic or organic UV-ray protective component, and in particular, an organic UV-ray protective component is preferably contained.

The inorganic UV-ray protective components may be components in which the aforementioned inorganic powder pigments, metal powder pigments and the like are blended as UV-ray dispersants. As examples thereof, mention may be made of metal oxides such as titanium oxide, zinc oxide, cerium oxide, titanium suboxide, iron-doped titanium oxides and the like; metal hydroxides such as iron hydroxides and the like; metal flakes such as platy iron oxide, aluminum flake and the like; and ceramics such as silicon carbide and the like. Among these, at least one type of a material selected from fine particulate metal oxides and fine particulate metal hydroxides with an average particle size ranging from 1 to 100 nm in the form of granules, plates, needles, or fibers is, in particular, preferred. The aforementioned powders are preferably subjected to conventional surface treatments such as fluorine compound treatments, among which a perfluoroalkyl phosphate treatment, a perfluoroalkylsilane treatment, a perfluoropolyether treatment, a fluorosilicone treatment, and a fluorinated silicone resin treatment are preferred; silicone treatments, among which a methylhydrogenpolysiloxane treatment, a dimethylpolysiloxane treatment, and a vapor-phase tetramethyltetrahydrogencyclotetrasiloxane treatment are preferred; silicone resin treatments, among which a trimethylsiloxysilicic acid treatment is preferred; pendant treatments which are methods of adding alkyl chains after the vapor-phase silicone treatment; silane coupling agent treatments; titanium coupling agent treatments; silane treatments among which an alkylsilane treatment and an alkylsilazane treatment are preferred; oil agent treatments; N-acylated lysine treatments; polyacrylic acid treatments; metallic soap treatments in which a stearic acid salt or a myristic acid salt is preferably used; acrylic resin treatments; metal oxide treatments and the like. Multiple treatments described above are preferably carried out. For example, the surface of the fine particulate titanium oxide can be coated with a metal oxide such as silicon oxide, alumina or the like, and then, a surface treatment with an alkylsilane can be carried out. The total amount of the material used for the surface treatment may preferably range from 0.1 to 50% by weight (mass) based on the amount of the powder.

The organic UV-ray protective components are generally lipophilic. More particularly, as examples of the aforementioned organic UV-ray protective components, mention may be made of benzoic acid-based UV-ray absorbers such as paraminobenzoic acid (hereinafter, referred to as PABA), PABA monoglycerol ester, N,N-dipropoxy-PABA ethyl ester, N,N-diethoxy-PABA ethyl ester, N,N-dimethyl-PABA ethyl ester, N,N-dimethyl-PABA butyl ester, 2-[4-(diethylamino)-2-hydroxybenzoyl]benzoic acid hexyl ester (trade name: Uvinul A Plus) and the like; anthranilic acid-based UV-ray absorbers such as homomethyl N-acetylanthranilate and the like; salicylic acid-based UV-ray absorbers such as amyl salicylate, menthyl salicylate, homomethyl salicylate, octyl salicylate, phenyl salicylate, benzyl salicylate, p-isopropanolphenyl salicylate and the like; cinnamic acid-based UV-ray absorbers such as octyl cinnamate, ethyl 4-isopropylcinnamate, methyl 2,5-diisopropylcinnamate, ethyl 2,4-diisopropylcinnamate, methyl 2,4-diisopropylcinnamate, propyl p-methoxycinnamate, isopropyl p-methoxycinnamate, isoamyl p-methoxycinnamate, octyl p-methoxycinnamate (2-ethylhexyl p-methoxycinnamate), 2-ethoxyethyl p-methoxycinnamate, cyclohexyl p-methoxy cinnamate, ethyl α-cyano-β-phenylcinnamate, 2-ethylhexyl α-cyano-β-phenylcinnamate, glyceryl mono-2-ethylhexanoyl-diparamethoxycinnamate, 3-methyl-4-[methylbis(trimethylsiloxy)silyl]butyl 3,4,5-trimethoxycinnamate, dimethicodiethyl benzal malonate (trade name: Parsol SLX (INCI name=polysilicone-15) and the like; benzophenone-based UV-ray absorbers such as 2,4-dihydroxybenzophenone, 2,2′-dihydroxy-4-methoxybenzophenone, 2,2′-dihydroxy-4,4′-dimethoxybenzophenone, 2,2′,4,4′-tetrahydroxybenzophenone, 2-hydroxy-4-methoxybenzophenone, 2-hydroxy-4-methoxy-4′-methylbenzophenone, 2-hydroxy-4-methoxybenzophenone 5-sulfonate, 4-phenylbenzophenone, 2-ethylhexyl-4′-phenylbenzophenone 2-carboxylate, hydroxy-4-n-octoxybenzophenone, 4-hydroxy-3-carboxybenzophenone and the like; 3-(4′-methylbenzylidene)-d,l-camphor; 3-benzylidene-d,l-camphor; urocanic acid; ethyl urocanate; 2-phenyl-5-methylbenzoxazole; benzotriazole-based UV-ray absorbers such as 2,2′-hydroxy-5-methylphenyl benzotriazole, 2-(2′-hydroxy-5′-t-octylphenyl)benzotriazole, 2-(2′-hydroxy-5′-methylphenyl)benzotriazole, dibenzaladine, dianisoylmethane, 4-methoxy-4′-t-butylbenzoylmethane, 5-(3,3-dimethyl-2-norbonylidene)-3-pentan-2-one, 2,2′-methylenebis(6-(2H-benzotriazol-2-yl)-4-(1,1,3,3-tetramethylbutyl)phenol) (trade name: trademark TINOSORB M) and the like; triazine-based UV-ray absorbers such as 2,4,6-tris[4-(2-ethylhexyloxycarbonyl)anilino]1,3,5-triazine (INCI: octyltriazone), 2,4-bis{[4-(2-ethyl-hexyloxy)-2-hydroxy]phenyl}-6-(4-methoxyphenyl)-1,3,5-triazine (INCI: bis-ethylhexyloxyphenol methoxyphenyl triazine, trade name: trademark TINOSORB S) and the like; 2-ethylhexyl 2-cyano-3,3-diphenylprop-2-enoate (INCI: octocrylene) and the like.

Furthermore, hydrophobic polymer powders containing the aforementioned organic UV-ray protective components inside thereof can also be used. The polymer powder may be hollow or not, may have an average primary particle size thereof ranging from 0.1 to 50 μm and may have a particle size distribution thereof of either broad or sharp. As examples of the polymers, mention may be made of acrylic resins, methacrylic resins, styrene resins, polyurethane resins, polyethylene, polypropylene, polyethylene terephthalate, silicone resins, nylons, acrylamide resins, and silylated polypeptide resins. Polymer powders containing the organic UV-ray protective components in an amount ranging from 0.1 to 30% by weight (mass) with respect to the amount of the powder are preferred. Polymer powders containing 4-tert-butyl-4′-methoxydibenzoylmethane, which is a UV-A absorber, are particularly preferred.

The aforementioned (J) UV-ray protective components which can be preferably used in the cosmetics for hair of the present invention may be at least one type of compound selected from the group consisting of fine particulate titanium oxide, fine particulate zinc oxide, 2-ethylhexyl paramethoxycinnamate, 4-tert-butyl-4′-methoxydibenzoylmethane, benzotriazole-based UV-ray absorbers and triazine-based UV-ray absorbers. The aforementioned (J) UV-ray protective components are commonly used and easily available, and exhibit superior effects of preventing ultraviolet rays. For these reasons, the aforementioned UV-ray protective components are preferably used. In particular, inorganic UV-ray protective components and organic UV-ray protective components are preferably used in combination. In addition, UV-A protective components and UV-B protective components are further preferably used in combination.

In the cosmetic for hair of the present invention, by use of the aforementioned (A) sugar alcohol-modified silicone together with the aforementioned (J) UV-ray protective component(s), the whole feeling on touch and storage stability of the cosmetic can be improved, and at the same time, the UV-ray protective component(s) can be stably dispersed in the cosmetic for hair. For this reason, superior UV-ray protective functions can be provided to the cosmetic.

In the cosmetic of the present invention, the aforementioned (J) UV-ray protective component(s) may be blended in a total amount preferably ranging from 0.1 to 40.0% by weight (mass), and more preferably ranging from 0.5 to 15.0% by weight (mass), with respect to the total amount of the cosmetic can be blended.

Oxidation Dye

In the case of using the cosmetic for hair of the present invention as an oxidation dye preparation, the cosmetic for hair of the present invention can comprise (K) an oxidation dye. As the aforementioned (K) oxidation dye, one which is generally used in an oxidation dye preparation such as an oxidation dye precursor, a coupler or the like can be used. For example, as examples of oxidation dye precursors, mention may be made of phenylene diamines, aminophenols, diaminopyridines, salts thereof such as hydrochloride salts, sulfate salts and the like. More particularly, as examples thereof, mention may be made of phenylenediamines such as p-phenylenediamine, toluene-2,5-diamine, toluene-3,4-diamine, 2,5-diaminoanisole, N-phenyl-p-phenylenediamine, N-methyl-p-phenylenediamine, N,N-dimethyl-p-phenylenediamine, 6-methoxy-3-methyl-p-phenylenediamine, N,N-diethyl-2-methyl-p-phenylenediamine, N-ethyl-N-(hydroxyethyl)-p-phenylenediamine, N-(2-hydroxypropyl)-p-phenylenediamine, 2-chloro-6-methyl-p-phenylenediamine, 2-chloro-p-phenylenediamine, N,N-bis-(2-hydroxyethyl)-p-phenylenediamine, 2,6-dichloro-p-phenylenediamine, 2-chloro-6-bromo-p-phenylenediamine and the like; aminophenols such as p-aminophenol, o-aminophenol, 2,4-diaminophenol, 5-aminosalicylic acid, 2-methyl-4-aminophenol, 3-methyl-4-aminophenol, 2,6-dimethyl-4-aminophenol, 3,5-dimethyl-4-aminophenol, 2,3-dimethyl-4-aminophenol, 2,5-dimethyl-4-aminophenol, 2-chloro-4-aminophenol, 3-chloro-4-aminophenol, and the like; diaminopyridines such as 2,5-diaminopyridine and the like; salts thereof; and the like. As examples of couplers, mention may be made of resorcinol, m-aminophenol, m-phenylenediamine, 2,4-diaminophenoxyethanol, 5-amino-o-cresol, 2-methyl-5-hydroxyethylaminophenol, 2,6-diaminopyridine, catechol, pyrogallol, gallic acid, tannic acid, and the like, as well as salts thereof. As other examples, those listed in “Japanese Standards of Quasi-drug Ingredients” (issued on June, 1991, by YAKUJI NIPPO LIMITED) can also be appropriately used. In addition, the aforementioned oxidation dye precursors and couplers can be used alone or in combination with two or more types thereof, and at least an oxidation dye precursor is preferably used. The blending amount of the oxidation dye preferably ranges from about 0.01 to 10% by weight (mass) of the total amount of the composition in view of dyeing properties and safety such as skin irritation or the like.

In the case of using the cosmetic for hair of the present invention as a double-agent type oxidation dye preparation, an alkaline agent and the aforementioned (K) oxidation dye (preferably further comprising a coupler) are contained in the first agent, and an oxidant is contained in the second agent, and at the time of use, the first agent and the second agent are mixed in a ratio usually ranging from 1:5 to 5:1, followed by using the mixture.

In the case of using the cosmetic for hair of the present invention as a hair bleaching preparation, the cosmetic for hair of the present invention can comprise the aforementioned oxidant. In the case of using the cosmetic for hair of the present invention as a double-agent type hair bleaching preparation, an alkaline agent is contained in the first agent, and an oxidant is contained in the second agent, and at the time of use, the first agent and the second agent are mixed in a ratio usually ranging from 1:5 to 5:1, followed by using the mixture.

Direct Dye

In the case of using the cosmetic for hair of the present invention as a temporary hair coloring preparation (such as a hair manicure), the cosmetic for hair of the present invention can comprise (L) a direct dye. As examples of direct dyes, mention may be made of, for example, a nitro dye, an anthraquinone dye, an acid dye, an oil-soluble dye, a basic dye and the like.

As examples of nitro dyes, mention may be made of HC Blue 2, HC Orange 1, HC Red 1, HC Red 3, HC Yellow 2, HC Yellow 4, and the like. As examples of anthraquinone dyes, mention may be made of 1-amino-4-methylaminoanthraquinone, 1,4-diaminoanthraquinone and the like.

As examples of acid dyes, mention may be made of Red No. 2, Red No. 3, Red No. 102, Red No. 104, Red No. 105, Red No. 106, Red No. 201, Red No. 227, Red No. 230, Red No. 232, Red No. 401, Red No. 502, Red No. 503, Red No. 504, Red No. 506, Orange No. 205, Orange No. 206, Orange No. 207, Yellow No. 4, Yellow No. 5, Yellow No. 202, Yellow No. 203, Yellow No. 402, Yellow No. 403, Yellow No. 406, Yellow No. 407, Green No. 3, Green No. 201, Green No. 204, Green No. 205, Green No. 401, Green No. 402, Blue No. 1, Blue No. 2, Blue No. 202, Blue No. 205, Violet No. 401, Black No. 401, Acid Blue 1, Acid Blue 3, Acid Blue 62, Acid Black 52, Acid Brown 13, Acid Green 50, Acid Orange 6, Acid Red 14, Acid Red 35, Acid Red 73, Acid Red 184, Brilliant Black 1 and the like.

As examples of oil-soluble dyes, mention may be made of Red No. 215, Red No. 218, Red No. 225, Orange No. 201, Orange No. 206, Yellow No. 201, Yellow No. 204, Green No. 202, Violet No. 201, Red No. 501, Red No. 505, Orange No. 403, Yellow No. 404, Yellow No. 405, Blue No. 403 and the like. For example, they are used in a coloring rinse, coloring treatment or the like.

As examples of basic dyes, mention may be made of Basic Blue 6, Basic Blue 7, Basic Blue 9, Basic Blue 26, Basic Blue 41, Basic Blue 99, Basic Blown 4, Basic Blown 16, Basic Blown 17, Basic Green 1, Basic Red 2, Basic Red 12, Basic Red 22, Basic Red 51, Basic Red 76, Basic Violet 1, Basic Violet 3, Basic Violet 10, Basic Violet 14, Basic Violet 57, Basic Yellow 57, Basic Yellow 87, Basic Orange 31, and the like. Among these, acid dyes are preferred, and in particular, Yellow No. 4, Yellow No. 203, Yellow No. 403, Orange No. 205, Green No. 3, Green No. 201, Green No. 204, Red No. 2, Red No. 104, Red No. 106, Red No. 201, Red No. 227, Blue No. 1, Blue No. 205, Violet No. 401, and Black No. 401 are preferred. The aforementioned (L) direct dyes can be used as one or more types thereof. The blending amount thereof in the cosmetic for hair of the present invention is not particularly restricted, and may preferably range from 0.005 to 5% by weight (mass) and more preferably range from 0.01 to 2% by weight (mass) with respect to the total weight (mass) of the composition.

In the case of using the cosmetic for hair of the present invention as a permanent waving preparation, the cosmetic for hair of the present invention can comprise the aforementioned reductant and oxidant. In the case of using the cosmetic for hair of the present invention as a double-agent type permanent waving preparation, for example, a reductant (preferably comprising an alkaline agent) is contained in the first agent and an oxidant is contained in the second agent. First, the first agent is applied to hair to dissociate disulfide bonds of the hair; subsequently, a preferable hair style is formed; subsequently, the second agent is applied thereto to reform the disulfide bonds of the hair; and thereby, a hair style may be fixed.

Other Components

In the cosmetics for hair of the present invention, (M) other components usually used in cosmetics for hair can be blended within a range which does not impair the effects of the present invention, such as organic resins, moisture-retaining agents, preservatives, anti-microbial agents, perfumes, salts, oxidants or antioxidants, pH adjusting agents, chelating agents, algefacients, anti-inflammatory agents, physiologically active components (such as whitening agents, cell activators, agents for ameliorating skin roughness, blood circulation accelerators, astringents, antiseborrheic agents and the like), vitamins, amino acids, nucleic acids, hormones, clathrate compounds, natural plant extract components, seaweed extract components, herb components, water, volatile solvents and the like. The other components are not particularly restricted thereto. They can be appropriately used alone or in combination with two or more types thereof.

As examples of organic resins, mention may be made of polyvinyl alcohol, polyvinyl pyrrolidone, poly(alkyl acrylate) copolymers, and the like. The organic resin possesses a superior property of forming a film. For this reason, by blending the organic resin in the cosmetic for hair of the present invention, a strong coating film can be formed at the applied part, and durability such as sebum resistance and rub resistance or the like can be improved.

As examples of humectants, mention may be made of, for example, hyaluronic acid, chondroitin sulfate, pyrrolidone carboxylic acid salts, polyoxyethylene methylglucoside, polyoxypropylene methylglucoside, and the like. Needless to say, the aforementioned polyhydric alcohols exhibit a function of retaining moisture on the skin or hair.

As examples of the preservatives, mention may be made of, for example, alkyl paraoxybenzoates, benzoic acid, sodium benzoate, sorbic acid, potassium sorbate, phenoxyethanol and the like. As examples of the antimicrobial agents, mention may be made of benzoic acid, salicylic acid, carbolic acid, sorbic acid, alkyl paraoxybenzoates, parachloromethacresol, hexachlorophene, benzalkonium chloride, chlorhexidine chloride, trichlorocarbanilide, trichlosan, photosensitizers, isothiazolinone compounds such as 2-methyl-4-isothiazolin-3-one, 5-chloro-2-methyl-4-isothiazolin-3-one and the like, amine oxides such as dimethyl laurylamine oxide, dihydroxyethyl laurylamine oxide and the like, and the like.

In addition, as examples of anti-microbial agents, mention may be made of apolactoferrin; phenol-based compounds such as resorcinol; anti-microbial or fungicidal basic proteins or peptides such as iturin-based peptides, surfactin-based peptides, protamine or salts thereof (protamine sulfate and the like) and the like; polylysines such as ε-polylysine or salts thereof, and the like; anti-microbial metal compounds which can produce a silver ion, a copper ion or the like; antimicrobial enzymes such as protease, lipase, oxydoreductase, carbohydrase, transferase, phytase and the like; and the like.

As examples of perfume, mention may be made of perfume extracted from flowers, seeds, leaves, and roots of various plants; perfume extracted from seaweeds; perfume extracted from various parts or secretion glands of animals such as musk and sperm oil; or artificially synthesized perfume such as menthol, musk, acetate, and vanilla. The conventional perfume can be selected and blended in an appropriate amount in accordance with the formulations of the cosmetics for hair in order to provide a certain aroma or scent to the cosmetics for hair, or in order to mask unpleasant odor.

As examples of oxidants, mention may be made of, for example, hydrogen peroxide, peroxidized urea, alkali metal salts of bromic acid, and the like. As examples of antioxidants, mention may be made of, for example, tocopherol, butylhydroxyanisole, dibutylhydroxytoluene, phytic acid and the like. As the antioxidants, ascorbic acid and/or ascorbic acid derivatives may be used. As examples of ascorbic acid derivatives which can be used, mention may be made of, for example, sodium ascorbate, potassium ascorbate, calcium ascorbate, ammonium ascorbate, erythorbic acid, sodium erythorbate, sodium ascorbyl phosphate, ascorbyl citrate, ascorbyl acetate, ascorbyl tartarate, ascorbyl palmitate, ascorbyl stearate, ascorbyl glucoside and the like. In addition, as the antioxidants, the reductants may be used. For example, sulfurous acid, bisulfurous acid, thiosulfuric acid, thiolactic acid, thioglycolic acid, L-cysteine, N-acetyl-L-cysteine and salts thereof can be appropriately used.

As examples of pH adjustors, mention may be made of, for example, lactic acid, citric acid, glycolic acid, succinic acid, tartaric acid, dl-malic acid, potassium carbonate, sodium hydrogencarbonate, ammonium hydrogencarbonate and the like. In addition, inorganic alkalized agents such as ammonia and the like, and organic alkalized agents such as isopropanolamine, monoethanolamine, diethanolamine, triethanolamine, 2-amino-2-methyl-1-propanolamine and the like can also be used. The blending amount of the pH adjustors is not particularly restricted, and may preferably range from 0.01 to 20% by weight (mass) and more preferably range from 0.1 to 10% by weight with respect to the total weight (mass) of the composition.

As examples of chelating agents, mention may be made of, for example, alanine, sodium edetate, sodium polyphosphate, sodium metaphosphate, phosphoric acid and the like.

As examples of algefacients, mention may be made of l-menthol, camphor and the like.

As examples of physiologically active components, mention may be made of, for example, vitamins, amino acids, nucleic acids, hormones, components extracted from natural vegetables, seaweed extracted components, herbal medicine components, whitening agents such as placenta extracts, arbutin, glutathione, saxifrageous extracts and the like; cell activators such as royal jelly, and the like; agents for ameliorating skin roughness; blood circulation accelerators such as nonylic acid vanillylamide, benzyl nicotinate, beta-butoxyethyl nicotinate, capsaicin, gingerone, cantharide tincture, ichthammol, caffeine, tannic acid, alpha-borneol, tocopherol nicotinate, inositol hexanicotinate, cyclandelate, cinnarizine, tolazoline, acetylcholine, verapamil, cepharanthine, gamma-orizanol and the like; astringents such as zinc oxide, tannic acid and the like; antiseborrheic agents such as sulfur, thianthol and the like; anti-inflammatory agents such as e-aminocaproic acid, glycyrrhizinic acid, R-glycyrrhetinic acid, lysozyme chloride, guaiazulene, hydrocortisone, allantoin, tranexamic acid, azulene and the like; and the like.

As examples of vitamins, mention may be made of vitamin As such as vitamin A oil, retinol, retinol acetate, retinol palmitate and the like; vitamin Bs such as vitamin B2s such as riboflavin, riboflavin butyrate, flavin adenine dinucleotide and the like; vitamin B6s such as pyridoxine hydrochloride, pyridoxine dioctanoate, pyridoxine tripalmitate and the like; vitamin B12 and derivatives thereof; vitamin B15 and derivatives thereof, and the like; vitamin Cs such as L-ascorbic acid, L-ascorbyl dipalmitic acid esters, sodium L-ascorbyl 2-sulfate, dipotassium L-ascorbyl phosphoric acid diester and the like; vitamin Ds such as ergocalciferol, cholecalciferol and the like; vitamin Es such as alpha-tocopherol, beta-tocopherol, gamma-tocopherol, dl-alpha-tocopherol acetate, dl-alpha-tocopherol nicotinate, dl-alpha-tocopherol succinate and the like; vitamin H; vitamin P; nicotinic acids such as nicotinic acid, benzyl nicotinate and the like; pantothenic acids such as calcium pantothenate, D-pantothenyl alcohol, pantothenyl ethyl ether, acetyl pantothenyl ethyl ether and the like; and the like.

As examples of amino acids, mention may be made of glycine, valine, leucine, isoleucine, serine, threonine, phenylalanine, arginine, lysine, aspartic acid, glutamate, cystine, cysteine, methionine, tryptophan and the like.

As examples of nucleic acids, mention may be made of deoxyribonucleic acid and the like.

As examples of hormones, mention may be made of estradiol, ethenyl estradiol and the like.

In the preparations for external use of the present invention, natural vegetable extracted components, seaweed extracted components and herbal medicine components can be blended in accordance with the purposes thereof. As the aforementioned components, in particular, one or more types of components having effects such as whitening effects, anti-ageing effects, effects of ameliorating ageing, effects of beautifying skin, anti-microbial effects, preservative effects and the like can be preferably blended.

As detailed examples thereof, mention may be made of, for example, Angelica keiskei extract, avocado extract, Hydrangea serrata extract, Althaea officinalis extract, Arnica montana extract, aloe extract, apricot extract, apricot kernel extract, Gingko biloba extract, fennel fruit extract, turmeric root extract, oolong tea extract, Rosa multiflora extract, Echinacea angustifolia leaf extract, Scutellaria baicalensis root extract, Phellodendron amurense bark extract, Coptis rhizome extract, Hordeum vulgare seed extract, Hypericum perforatum extract, Lamium album extract, Nasturtium officinale extract, orange extract, dried sea water solution, seaweed extract, hydrolyzed elastin, hydrolyzed wheat powders, hydrolyzed silk, Chamomilla recutita extract, carrot extract, Artemisia capillaris flower extract, Glycyrrhiza glabra extract, Hibiscus sabdariffa extract, Pyracantha fortuneana extract, kiwi extract, Cinchona succirubra extract, cucumber extract, guanosine, Gardenia florida extract, Sasa veitchii extract, Sophora angustifolia extract, walnut extract, grapefruit extract, Clematis vitalba leaf extract, chlorella extract, Morus alba extract, Gentiana lutea extract, black tea extract, yeast extract, burdock extract, fermented rice bran extract, rice germ oil, Symphytum officinale leaf extract, collagen, Vaccinum vitis idaea extract, Asiasarum sieboldi extract, Bupleurum falcatum extract, umbilical extract, Salvia extract, Crocus sativus flower extract, sasa bamboo grass extract, Crataegus cuneata fruit extract, Zanthoxylum piperitum extract, Corthellus shiitake extract, Rehmannia chinensis root extract, Lithospermum erythrorhizone root extract, Perilla ocymoides extract, Tilia cordata extract, Spiraea ulmaria extract, Paeonia albiflora extract, Acorns calamus root extract, Betula alba extract, Equisetum arvense extract, Hedera helix extract, Crataegus oxyacantha extract, Sambucus nigra extract, Achillea millefolium extract, Mentha piperita leaf extract, sage extract, Malva sylvestris extract, Cnidium officinale root extract, Swertia japonica extract, soybean seed extract, Zizyphus jujuba fruit extract, thyme extract, Camellia sinensis leaf extract, Eugenia caryophyllus flower extract, Imperata cylindrica extract, Citrus unshiu peel extract, Angelica acutiloba root extract, Calendula officinalis extract, Prunus persica kernel extract, Citrus aurantium peel extract, Houttuynia cordata extract, tomato extract, natto extract, carrot extract, garlic extract, Rosa canina fruit extract, hibiscus extract, Ophiopogon japonicus root extract, Nelumbo nucifera extract, parsley extract, honey, Hamamelis virginiana extract, Parietaria officinalis extract, Isodon trichocarpus extract, bisabolol, Eriobotrya japonica extract, Tussilago farfara flower extract, Petasites japonicus extract, Poria cocos extract, Ruscus aculeatus root extract, grape extract, propolis, Luffa cylindrica fruit extract, safflower flower extract, peppermint extract, Tillia miquellana extract, Paeonia suffruticosa root extract, Humulus lupulus extract, Pinus sylvestris cone extract, horse chestnut extract, Lysichiton camtschatcense extract, Sapindus mukurossi peel extract, Melissa officinalis leaf extract, peach extract, Centaurea cyanus flower extract, Eucalyptus globulus leaf extract, Saxifraga sarementosa extract, Citrus junos extract, Coix lacryma-jobi seed extract, Artemisia princeps extract, lavender extract, apple extract, lettuce extract, lemon extract, Astragalus sinicus extract, rose extract, rosemary extract, Roman chamomile extract, royal jelly extract, and the like. The aforementioned extracts may be water-soluble or oil-soluble.

The cosmetic for hair of the present invention may further comprise water. Therefore, the preparation for external use of the present invention can be in the form of an oil-in-water emulsion or a water-in-oil emulsion. In this case, the cosmetic for hair of the present invention exhibits superior emulsion stability and a superior sensation during use.

Water is not particularly restricted as long as it does not include any harmful components for human bodies and is clean. As examples thereof, mention may be made of tap water, purified water, and mineral water. In addition, in the cosmetic for hair, and in particular, the cosmetic for hair in the form of an emulsion composition of the present invention, the blending amount of water preferably ranges from 2 to 98% by weight (mass), with respect to the total weight (mass) of the cosmetic.

In the cosmetic for hair of the present invention, depending on the formulations and the purposes thereof, volatile solvents such as light isoparaffins, ethers, LPG, N-methylpyrrolidone, next-generation chlorofluorocarbons, and the like, can be blended in addition to water.

The aforementioned (A) sugar alcohol-modified silicone may be blended in a cosmetic composition for hair, as it is, or alternatively, may be blended therein as an emulsion obtained by using water and a surfactant of the aforementioned component (C) beforehand. In addition, an emulsion may be produced by using an oil agent of the aforementioned component (B) or a part thereof, water and the surfactant of the aforementioned component (C), in addition to the aforementioned (A) sugar alcohol-modified silicone, and then the emulsion may be blended in a cosmetic composition for hair. The form of the emulsion must be adapted with the form of the cosmetic composition for hair to be blended. For example, in the case of a hair cleansing cosmetic in the form of an oil-in-water emulsion, if the same type of oil-in-water emulsion of the (A) sugar alcohol-modified silicone is prepared, the emulsion can be blended in the cosmetic as it is. In this case, as the surfactant of the aforementioned component (C) used in the preparation for the emulsion of the aforementioned (A) sugar alcohol-modified silicone, an appropriate one is preferably selected in order to maintain stability of the blending system. The surfactants of the aforementioned component (C) may be a combination of plural types of surfactants, and different types of surfactants such as ionic surfactants, nonionic surfactants and the like can be used together in order to ensure stability of the emulsion.

The form of the emulsion may be not only an oil-in-water emulsion or water-in-oil emulsion, but also a multiple emulsion or microemulsion thereof. The form of the emulsion (oil-in-water type or water-in-oil type) and the particle size of the emulsion can be appropriately selected or adjusted.

In the case of the cosmetic for hair of the present invention is in the form of an oil-in-water emulsion, the dispersion phase of the aforementioned cosmetic is formed from particles obtained by emulsifying the aforementioned (A) sugar alcohol-modified silicone or a mixture of the aforementioned (B) oil agent therewith by means of the surfactant of the aforementioned component (C). The average particle size thereof can be measured by a conventional measurement device using a laser diffraction/scattering method or the like. The cosmetic in the form of an oil-in-water emulsion may be a transparent microemulsion in which the average particle size of the dispersion phase measured is 0.1 μm or less, or may be a milky emulsion having a large particle size so that the average particle size exceeds 4 μm. In addition, in order to improve stability and transparency of the outer appearance of the emulsion, the emulsion particles can be miniaturized. In particular, in order to improve the adhesive property with respect to the hair or skin or a sensation during use, an emulsion having an average particle size ranging from 0.5 to 20 μm can be selected, and is preferred. For example, in the case of a microemulsion, stability is improved, and in the case of a cleansing cosmetic, foam quality is improved. In the case of a normal particle size ranging from submicrons to 4 μm, superior usability is exhibited, good balance between a blending effect and stability is exhibited, and preparation is easily carried out. In addition, in the case of a large particle size of several microns or more, and for example, ranging from 4 to 5 μm, improvements of adhesive properties to hair and a sensation during use may be expected.

The cosmetic for hair of the present invention in the form of an oil-in-water emulsion or a water-in-oil emulsion can be produced by mixing components of the aforementioned cosmetic using a mechanical force by means of an apparatus such as a homomixer, a paddle mixer, a Henschel mixer, a homodisper, a colloid mill, a propeller stirrer, a homogenizer, an in-line type continuous emulsifier, an ultrasonic emulsifier, a vacuum kneader or the like.

The cosmetic for hair of the present invention in the form of an emulsion essentially comprises the aforementioned (A) sugar alcohol-modified silicone, and superior dispersion stability of a dispersion phase can be obtained. Therefore, the cosmetics for hair of the present invention exhibit superior stability over time, possess a uniform outer appearance, and provide a superior sensation during use.

The forms of the cosmetics for hair of the present invention are not particularly restricted, and may be in the form of liquids, creams, solids, pastes, gels, powders, lamellas, mousses, sprays, sheets, and the like, in addition to emulsions.

The cosmetic compositions for hair of the present invention include all usages for cosmetics to be applied on hair. In particular, the cosmetics of the present invention are preferably used in cosmetics for cleansing hair, cosmetics for conditioning hair, cosmetics for styling hair, and cosmetics for dyeing hair.

The cosmetics for cleansing hair are cleansing preparations used in order to wash and clean hair and/or scalp. The functions are diverse and in addition to a base function of cleansing, additional functions such as conditioning effects, effects of preventing dandruff, and the like may be possessed. More particularly, as examples thereof, mention may be made of shampoos, conditioning shampoos, anti-dandruff shampoos, and the like.

The cosmetics for conditioning hair are cosmetics for hair possessing functions of concealing damage of hair, repairing damage of hair, protecting hair from damage, or preventing damage of hair, and the like. The hair conditioning cosmetics may be applied immediately after cleansing hair or after drying hair. More particularly, as examples thereof, mention may be made of rinses, rinse-in-shampoos, hair conditioners, hair creams, hair treatments and the like.

The cosmetics for styling hair are cosmetics for the purpose of finishing hair, and are roughly divided into a type of mainly styling hair such as fixing and setting hair, and another type of mainly improving glossiness, a feeling on touch, texture, and easiness of handling of hair. By virtue of multifunctionalization and sophistication of cosmetics, some cosmetics possess both of the aforementioned functions. Some hair-styling cosmetics may exhibit functions overlapped with those of the cosmetics for conditioning hair. More particularly, as examples thereof, mention may be made of hair foams, hair sprays, hair styling lotions, hair gels, hair liquids, hair oils, hair waxes, preparations for use in blowing hair, and the like. In particular, as examples thereof, mention may be made of hair mists, super hard mousse, super hard gels, super hard sprays, hard mousse, hard gels, hard sprays, soft sprays, soft mousse, soft gels, lotions for use in blowing hair, lotions for use in straightening hair, mousse for use in straightening hair, water, pomades, hair liquids, wet gels, hair waxes, hair creams, hair milks, mousse for waving hair, styling essences and the like.

The cosmetics of dyeing hair are for temporarily, semi-temporarily or permanently coloring hair by physically or chemically acting on the surface of hair. As examples thereof, mention may be made of color sprays, color sticks, hair manicures, coloring lotions, gloss sprays, manicure sprays and the like.

The cosmetic compositions for hair of the present invention can comprise any combinations of the aforementioned optional components as long as the aforementioned (A) sugar alcohol-modified silicone is contained. Namely, the cosmetic compositions for hair of the present invention can comprise any combinations of the aforementioned (A) sugar alcohol-modified silicone and at least any one of the following components (B) to (M).

(B) Oil agents

### (C) Surfactants

(D) Water-soluble polymers

### (E) Alcohols

(F) Thickening and/or gelling agents

### (G) Powders

(H) Solid silicone resins or crosslinking organopolysiloxanes
(I) Acryl silicone dendrimer copolymers
(J) UV-ray protective components
(K) Oxidation hair dyes
(L) Direct dyes
(M) Organic resins, moisture-retaining agents, preservative, anti-microbial agents, perfumes, salts, oxidants or antioxidants, pH adjusting agents, chelating agents, algefacients, anti-inflammatory agents, physiologically active components (such as whitening agents, cell activators, agents for ameliorating skin roughness, blood circulation accelerators, astringents, antiseborrheic agents and the like), vitamins, amino acids, nucleic acids, hormones, clathrate compounds, natural plant extract components, seaweed extract components, herb components, water, volatile solvents and the like.

Among combinations of components (B) to (M), preferable combinations of the components for the cosmetics for hair of the present invention are described below.

(B)+{at least one selected from the group consisting of (C), (D), (E), (F), (G), (H), (I), (J), (K), (L) and (M)};
(B)+(C)+{at least one selected from the group consisting of (D), (E), (F), (G), (H), (I), (J), (K), (L) and (M)};
(B)+(C)+(D)+{at least one selected from the group consisting of (E), (F), (G), (H), (I), (J), (K), (L) and (M)};
(B)+(C)+(E)+{at least one selected from the group consisting of {(D), (F), (G), (H), (I), (J), (K), (L) and (M)};
(B)+(C)+(F)+{at least one selected from the group consisting of (D), (E), (G), (H), (I), (J), (K), (L) and (M)};
(B)+(C)+(D)+(E)+{at least one selected from the group consisting of (F), (G), (H), (I), (J), (K), (L) and (M)};
(B)+(C)+(D)+(F)+{at least one selected from the group consisting of (E), (G), (H), (I), (J), (K), (L) and (M)};
(B)+(C)+(D)+(E)+(F)+{at least one selected from the group consisting of (G), (H), (I), (J), (K), (L) and (M)};
(C)+{at least one selected from the group consisting of (B), (D), (E), (F), (G), (H), (I), (J), (K), (L) and (M)};
(C)+(D)+{at least one selected from the group consisting of (B), (E), (F), (G), (H), (I), (J), (K), (L) and (M)};
(C)+(E)+{at least one selected from the group consisting of (B), (D), (F), (G), (H), (I), (J), (K), (L) and (M)};
(C)+(F)+{at least one selected from the group consisting of (B), (D), (E), (G), (H), (I), (J), (K), (L) and (M)};
(C)+(D)+(E)+{at least one selected from the group consisting of (B), (F), (G), (H), (I), (J), (K), (L) and (M)};
(C)+(D)+(F)+{at least one selected from the group consisting of (B), (E), (G), (H), (I), (J), (K), (L) and (M)};
(C)+(E)+(F)+{at least one selected from the group consisting of (B), (D), (G), (H), (I), (J), (K), (L) and (M)}; and
(C)+(D)+(E)+(F)+{at least one selected from the group consisting of (B), (G), (H), (I), (J), (K), (L) and (M)}.

The cosmetics for hair of the present invention generally comprise water.

Hereinafter, generally preferable combinations and the blending purposes thereof are described in detail, in accordance with types and usages of cosmetics to be applied on hair. It should be understood that the cosmetic compositions for hair according to the present invention are not restricted to the detailed compositions.

Among cosmetics for hair of the present invention, a cosmetic for cleansing hair comprises, in addition to the aforementioned (A) sugar alcohol-modified silicone, the aforementioned (B) oil agent as a conditioning agent, the aforementioned (D) water-soluble polymer as a conditioning agent, the aforementioned (C) surfactant as a foaming and/or cleansing base agent, the aforementioned (E) alcohol as a humectant and/or a stabilizing agent, and the aforementioned (M) other components such as water, a pH adjustor, a preservative and the like, as representative components. In view of cleansing effects and the like, among components (C), (C1) an anionic surfactant is, in general, preferably used, and at least one surfactant selected from (C3) a nonionic surfactant and (C4) an amphoteric surfactant is, in particular, preferably used together therewith. In addition, as the aforementioned (B) oil agent, one or more types selected from organo-modified silicones such as dimethylpolysiloxanes, amino-modified silicones and the like, ester oils, lanolin derivatives and higher alcohols are preferably used. In particular, in view of conditioning effects to hair, use of amino-modified silicones is preferred, and the amino equivalence and the like of the aforementioned modified silicones can be appropriately designed. In the same manner as described above, among the aforementioned components (D), in view of conditioning effects, use of (D1) a cationic water-soluble polymer is preferred. In particular, in the case of using the aforementioned (A) sugar alcohol-modified silicone together with the aforementioned (C1) anionic surfactant and (D1) cationic water-soluble polymer, there are advantages in that superior foaming properties and a superior feeling on touch of foam can be obtained, superior cleansing properties can be exhibited, and smooth combability without a frictional sensation can be provided both at the time of wetting and at the time of drying after hair is cleaned.

Among the cosmetic compositions for hair of the present invention, a cosmetic for conditioning hair may preferably comprise, in addition to the aforementioned (A) sugar alcohol-modified silicone, the aforementioned (B) oil agent, and in particular, (B2-1) a higher alcohol, the aforementioned (C) surfactant, (E) alcohols, (D) water-soluble polymer (for example, as an aqueous thickening agent), and the aforementioned (M) other components such as water, pH adjustor, preservative, and the like, as representative blending components. In view of adhesive properties to hair, among the aforementioned components (C), use of (C2) a cationic surfactant as an essential component may, in general, be preferred. As examples thereof, mention may be made of a quaternary ammonium salt such as alkyltrimethylammonium chloride or the like or an alkylamidoamine such as diethylaminoethylamide stearate or the like. In addition, as the aforementioned (B) oil agent, one or more types selected from organo-modified silicones such as dimethylpolysiloxanes, amino-modified silicones and the like, ester oils, lanolin derivatives and higher alcohols are preferably used. In particular, use of the higher alcohols is preferred in view of forming an alpha gel as a surfactant.

In addition, in view of retaining properties on hair and conditioning effects for hair, use of silicones is preferred, and selection from amino-modified silicones or dimethylpolysiloxanes with a high degree of polymerization is also preferred. In particular, use of silicones with a high degree of polymerization which are silicone gums is preferred. The amino equivalence or the like of the aforementioned modified silicones can be appropriately designed. In addition, in order to emulsify the aforementioned silicones, use of one or more types selected from (C3) nonionic surfactants and (C4) amphoteric surfactants, other than cationic surfactants, is preferred. The aforementioned (D) water-soluble polymer may be preferably blended. In this case, as examples of the aforementioned component (D), mention may be made of water-soluble polymers other than cationic water-soluble polymers. In view of conditioning effects, use of natural water-soluble polymers such as guar gum and the like, semi-synthesized water-soluble polymers such as hydroxyethylcellulose and the like may, in particular, be preferred. On the other hand, in the case of using the aforementioned (A) sugar alcohol-modified silicone together with the aforementioned (B2-1) higher alcohol, and (C2) cationic surfactant, the cosmetic for conditioning hair can provide smooth combability without a frictional sensation both at the time of wetting and at the time of drying. At the time of drying, while smooth combability with a comb and fingers and a moisturizing feeling on touch can be exhibited, an uncomfortable sticky sensation is not exhibited and in addition, a flexible styling sensation can be provided to hair. In addition, the cosmetics for conditioning hair of the present invention exhibit superior durability of the aforementioned effects.

Among the cosmetic compositions for hair, the cosmetic for styling hair may comprise, in addition to the aforementioned (A) sugar alcohol-modified silicone, the aforementioned (B) oil agent, (C) surfactant, and (D) water-soluble polymer as essential components. The cosmetics for styling hair of the present invention may have oil-based raw materials as a base material or may have aqueous raw materials as a base material (namely, having (M) water as a carrier), and the base material therefor is not particularly restricted. The cosmetic for styling hair of the present invention may preferably comprise an oil agent as the aforementioned component (B). The compositions and blending components may be determined in accordance with the formulation selected from a liquid, a cream, a solid, a paste, a gel, a mousse, and a spray. In the case of blending the aforementioned (A) sugar alcohol-modified silicone of the present invention, smooth combability with a comb or fingers at the time of drying can be exhibited, a flexible styling sensation may be provided to hair, and superior durability of the aforementioned styling effects can be exhibited.

Use of the aforementioned (B) oil agent with a high degree of viscosity which is in the form of a wax or a gum at room temperature (25° C.), together with the aforementioned (B) oil agent which is in the form of a liquid at room temperature is preferred. In particular, use of a combination between an oil agent with a high degree of viscosity having 5,000 mPa·s or more at room temperature (more preferably an oil agent with a viscosity of 10,000 mPa·s or more to an oil agent in the form of a solid) and an oil agent with a low degree of viscosity having less than 5,000 mPa·s at room temperature (more preferably in the range of 0.65 to 3,000 mPa·s) is preferred. In addition, as the aforementioned component (D), use of a vinyl-based polymer such as polyvinylpyrrolidone, carboxyvinyl polymer or the like together with another water-soluble polymer is also preferred.

Among cosmetic compositions for hair, the cosmetic of dyeing hair may comprise, in addition to the aforementioned (A) sugar alcohol-modified silicone, one or more types of hair dyeing components selected from the aforementioned (K) oxidation hair dyes and (L) direct dyes. In particular, by using the aforementioned (A) sugar alcohol-modified silicone according to the present invention together with the aforementioned hair dyeing components, there can be advantages in that dispersing properties and stability of the hair dyeing components can be improved, color durability and development on hair can be enhanced, uneven coloring can be overcome, and hair can be beautifully dyed. In addition, in the case of using the aforementioned (L) direct dye, there can be an advantage in that it is relatively easy to rinse off the composition, if necessary.

### EXAMPLES

Hereinafter, the present invention is described in detail with reference to examples. It should be understood that the present invention is not restricted to the examples. In the composition formulae described below, an Me₃SiO group (or a Me₃Si group) is indicated as “M”, an Me₂SiO group is indicated as “D”, an Me₂HSiO group is indicated as “M^{H}”, an MeHSiO group is indicated as “D^{H}”, and units in which a methyl group (Me) in M and D is modified by any substituent are respectively indicated as “M^{R}” and “D^{R}”.

In addition, the xylitol monoallyl ether and the xylitol residue described in the following Synthesis Examples 1 to 3 are the same raw material and functional group as described in the specification of the present application. More particularly, the xylitol monoallyl ether is a raw material comprising xylitol monoallyl ethers represented by the following structural formula: CH₂═CH—CH₂—OCH₂[CH(OH)]₃CH₂OH and represented by the following structural formula: CH₂═CH—CH₂—OCH{CH(OH)CH₂OH}₂ in a weight (mass) ratio of 9:1. In the sugar alcohol-modified silicone obtained in Synthesis Examples 1 to 3, the xylitol residue of C₃H₆—OCH₂[CH(OH)]₃CH₂OH or —C₃H₆—OCH{CH(OH)CH₂OH}₂ corresponding thereto is introduced in the same weight (mass) ratio as described above.

### Synthesis Example 1

### Synthesis of Silicone Compound No. 1

197.2 g of a methylhydrogenpolysiloxane represented by the following average compositional formula: M^{H}D₄₀₀M^{H}, 2.8 g of a xylitol monoallyl ether, and 200 g of isopropyl alcohol (IPA) were placed in a reactor, and the mixture was heated to 70° C. under a nitrogen stream while it was stirred. 0.060 g of a solution of a platinum-1,3-divinyl-1,1,3,3-tetramethyldisiloxane complex (Pt concentration=4.5% by weight (mass)) dissolved in IPA was added thereto, and the mixture was reacted for 5 hours at 80° C. Subsequently, with 2 g of the reaction mixture, it was confirmed that the reaction was completed, by means of an alkaline decomposition gas generation method (remaining Si—H group was decomposed by an aqueous solution/ethanol of KOH, and reaction index was calculated from the volume of generated hydrogen gas). 200.0 g of a dimethylpolysiloxane (2 cst, 25° C.) was added to the reaction mixture in order to dilute the reaction mixture. Subsequently, the reaction mixture was heated under reduced pressure to remove low-boiling components other than the diluent by distillation. Thereby, a xylitol-modified silicone represented by the following average compositional formula: M^{R}*²¹D₄₀₀M^{R}*²¹, wherein R^{*21}=a hydrophilic group represented by —C₃H₆O—X in which X is a xylitol moiety, (a mixture consisting of a composition containing Silicone Compound No. 1 and a dimethylpolysiloxane (2 cSt, 25° C.; diluent)) was obtained. The weight (mass) ratio of the aforementioned silicone compound:diluent was 1:1. The product was in the form of a brownish gray-white, uniform and viscous liquid.

### Synthesis Example 2

### Synthesis of Silicone Compound No. 2

159.5 g of a methylhydrogenpolysiloxane represented by the following average compositional formula: MD₇₂D^{H}₁₂M, 81.9 g of a vinyltristrimethylsiloxysilane represented by the following average compositional formula: CH₂═CH—Si(OSiMe₃)₃, 19.8 g of xylitol monoallyl ether, and 75 g of isopropyl alcohol (IPA) were placed in a reactor, and the mixture was heated to 80° C. under a nitrogen stream while it was stirred. mg of a platinum catalyst was added thereto, and the mixture was reacted for 2.5 hours at 80° C. It was confirmed that the Si—H bond had disappeared by means of an IR spectrum, and the reaction had proceeded. The reaction mixture was heated under reduced pressure to remove low-boiling components by distillation. Thereby, a xylitol-co-modified silicone having a siloxane dendron structure represented by the following average compositional formula: MD₇₂D^{R}*²¹₃D^{R}*³¹₉M, wherein R^{*21}=a hydrophilic group represented by —C₃H₆O—X in which X is a xylitol moiety; and R^{*31}=—C2H₄Si(OSiMe₃)₃, was obtained. The yield was 222.0 g (85%). The obtained product was a brownish gray-white and very viscous liquid having a kinetic viscosity at 25° C. of 298,900 mm²/sec, and a refractive index of 1.416.

### Synthesis Example 3

### Synthesis of Silicone Compound No. 3

184.0 g of a methylhydrogenpolysiloxane represented by the following average compositional formula: MD₄₀₀D^{H}₁₀M, 7.0 g of a vinyltristrimethylsiloxysilane represented by the following average compositional formula: CH₂═CH—Si(OSiMe₃)₃, 7.0 g of xylitol monoallyl ether, 200 g of IPA, and 0.16 g of a solution containing 2.3% by weight (mass) of sodium acetate dissolved in methanol were placed in a reactor, and the mixture was heated to 75° C. under a nitrogen stream while it was stirred. 0.06 g of a solution containing 5% by weight (mass) of chloroplatinic acid dissolved in IPA was added thereto, and the mixture was reacted for 2 hours at 80° C. Subsequently, with 2 g of the reaction mixture, it was confirmed that the reaction had proceeded 85%, by means of an alkaline decomposition gas generation method. Subsequently, 1.1 g of 1-decene and 0.06 g of a solution containing 5% by weight (mass) of chloroplatinic acid dissolved in IPA were added thereto, and the mixture was reacted for 3 hours at 80° C. Subsequently, with a small amount of the reaction mixture, it was confirmed that the reaction was completed, by means of an alkaline decomposition gas generation method. 198.0 g of a dimethylpolysiloxane (2 cst, 25° C.) was added to the reaction mixture to dilute the reaction mixture. Subsequently, the reaction mixture was heated under reduced pressure to remove low-boiling components other than the diluent by distillation. Thereby, a xylitol-modified silicone having an alkyl group and a siloxane dendron structure represented by the following average compositional formula: MD₄₀₀D^{R}*¹¹₂D^{R}*³¹₃D^{R}*²¹₅M, wherein R*²¹ and R*³¹ are the same as defined above; and R*¹¹=—C10H₂₁, (a mixture consisting of a composition containing Silicone Compound No. 3 and a dimethylpolysiloxane (2 cst, 25° C.; diluent)) was obtained. The weight (mass) ratio of the aforementioned silicone composition:diluent was 1:1. The mixture was in the form of a brownish slightly gray-white, and viscous liquid.

### Comparative Synthesis Example 1

### Synthesis of Silicone Compound RE 1

111.6 g of a methylhydrogenpolysiloxane represented by the following average compositional formula: MD₆₁D^{H}₁₅M was placed in a reactor. A mixture consisting of 30.9 g of a dimethylpolysiloxane of which one terminal was modified with a vinyl group represented by the following structural formula: CH₂═CHSiMe₂(OSiMe₂)₆OSiMe₃ and 0.10 g of a solution of a platinum-1,3-divinyl-1,1,3,3-tetramethyldisiloxane complex (Pt concentration=0.5% by weight (mass)) dissolved in toluene was added dropwise thereto, followed by stirring the mixture at room temperature. Thereby, a linear siloxane branched type polysiloxane intermediate was obtained.

In addition, 7.0 g of triglycerol monoallyl ether, 50.4 g of 1-dodecene, 100 g of IPA and 0.40 g of a solution of a platinum-1,3-divinyl-1,1,3,3-tetramethyldisiloxane complex (Pt concentration=0.5% by weight (mass)) dissolved in toluene were placed in another reactor. While the mixture was stirred under a nitrogen stream, the above-synthesized linear siloxane branched type polysiloxane intermediate was added dropwise thereto under refluxing the solvent. After completion of the dropwise addition, the mixture was heated and stirred for 3 hours. With 2 g of the reaction mixture, it was confirmed that the reaction was completed, by means of an alkaline decomposition gas generation method. The reaction mixture was heated under reduced pressure to remove low-boiling components by distillation. In addition, filtration was carried out. Thereby, an alkyl/linear siloxane/polyglycerol-co-modified silicone represented by the following average compositional formula: MD₆₁D^{R}*¹²₁₂D^{R}*⁴¹₂D^{R}*²²₁M, wherein R*¹²=—C₁₂H₂₅; R*⁴¹=—C2H₄SiMe₂(OSiMe₂)₆OSiMe₃; and R*²²=—C₃H₆O—X, wherein X is a triglycerol moiety, was obtained. The obtained product was in the form of an almost colorless, translucent, and uniform liquid.

### Comparative Synthesis Example 2

### Synthesis of Silicone Compound RE 2

241 g of a silicone of which both terminals were modified with phenol (average structural formula: M^{P}D₆₀M^{P}; and P=C₃H₆Ph-OH) was placed in a flask, and 9.6 g of a 30% methanol solution of potassium methoxide was added thereto. The mixture was heated to 60° C. under reduced pressure while stirring, so that all methanol was removed by distillation. Thereby, a potassiumated phenol-modified silicone was obtained as a yellow oil product. The product was heated to 95° C., and 22.2 g (3 equivalents) of glycidol was added thereto by means of a dropping funnel over 4 hours under a nitrogen stream while it was stirred. Subsequently, the reaction mixture was continuously heated and stirred for 2 hours, followed by cooling to room temperature. In addition, 500 mL of ethanol was added thereto to dilute the mixture, and potassium was removed by means of a cation-exchange resin, followed by concentrating. Thereby, a polyglycerol-modified silicone having an average compositional formula: M^{R}*²³D₆₀M^{R}*²³ was obtained as a pale yellow viscous liquid. In the aforementioned formula, R*²³ represents the following formula:

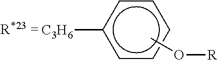

wherein R represents a triglycerol moiety.

Average compositional formulae of “Silicone Compound No. 1” to “Silicone Compound No. 3” used in Examples and “Silicone Compound RE 1” and “Silicone Compound RE 2” used in Comparative Examples are shown in Table 2.

TABLE 2

| Silicone | Average compositional | |
|---|---|---|
| Compound | formula | Property |
| Mixture | M^{R}*²¹D₄₀₀M^{R}*²¹ | Brownish gray- |
| containing | (diluted with | white, uniform |
| Silicone | dimethylpolysiloxane to a | and viscous |
| Compound No. 1 | 50% concentration) | liquid |
| Mixture | MD₇₂D^{R}*³¹₉D^{R}*²¹₃M | Brownish gray- |
| containing | (diluted with | white, and very |
| Silicone | dimethylpolysiloxane to a | viscous liquid |
| Compound No. 2 | 50% concentration) | |
| Mixture | MD₄₀₀D^{R}*¹¹₂D^{R}*³¹₃D^{R}*²¹₅M | Brownish slightly |
| containing | (diluted with | gray-white, |
| Silicone | dimethylpolysiloxane to a | uniform and |
| Compound No. 3 | 50% concentration) | viscous liquid |
| Silicone | MD₆₁D^{R}*¹²₁₂D^{R}*⁴¹₂ D^{R}*²²₁M | Almost colorless, |
| Compound RE 1 | | translucent, and |
| | | uniform liquid |
| Silicone | M^{R}*²³D₆₀M^{R}*²³ | Pale yellow |
| Compound RE 2 | | viscous liquid |

In the table, the structures and classifications thereof are described below.

<Long Chain Alkyl Group: R*¹>

R*¹¹=—C₁₀H₂₁

R*¹²=—C12H₂₅

### <Hydrophilic Group: R*²>

R*²¹=hydrophilic group represented by —C₃H₆O—X, wherein X represents a xylitol moiety.
R*²²=hydrophilic group represented by —C₃H₆O—X, wherein X represents a triglycerol moiety.

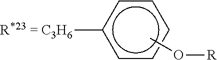

wherein R represents a triglycerol moiety.

### <Group Having a Siloxane Dendron Structure: R*³>

R*³¹=—C2H₄Si(OSiMe₃)₃

### <Group Having a Linear Polysiloxane Structure: R*⁴>

R*⁴¹=—C2H₄SiMe₂(OSiMe₂)₆OSiMe₃

### Examples 1 to 3 and Comparative Examples 1 and 2

Hair conditioners were prepared with the compositions shown in Table 3. Evaluation thereof was carried out on the basis of the evaluation criteria described below, and effects thereof were quantified.

First, as usage effects during wetting, the following categories were evaluated and pointed.
(A) Sensation during use at the time of applying on hair (smoothness during spreading and lightness or heaviness): 5 stages of 1 to 5 points
(B) Smoothness at the time of rinsing in running water: 5 stages of 1 to 5 points
(C) Feeling on touch (refreshing light smoothness sensation) at the time of drying with a towel: 5 stages of 1 to 5 points
   Subsequently, as usage effects during drying, the following category was evaluated and pointed.
(D) Conditioning effects after drying (refreshing light combability with fingers): 5 stages of 3, 6, 9, 12 and points

The total points for usage effects during wetting are 15 points and the total points for usage effects during drying are also 15 points. The results are also shown in Table 3. In the table, the numerical value described after each component indicates part(s) by weight (mass).

The evaluation methods for the aforementioned sensation during use at the time of applying to hair, smoothness at the time of rinsing in running water, a feeling on touch at the time of drying with a towel, and effects of conditioning after drying, as well as evaluation criteria are described below.

(A) Sensation During Use at the Time of Applying to Hair

A commercially available bundle of Chinese hair (manufactured by Beaulax Co., Ltd., 30 cm, 4 g) was subjected to a bleaching treatment for 10 minutes at room temperature, followed by cleansing the bundle with a 10% solution of sodium laureth sulfate. Subsequently, a sample (hair conditioner), in an amount of 1.0 g, was applied thereto. At the time of application, 1.0 g of the sample was put on the palm of a hand, and lightly spread thereon, followed by applying the sample from the roots to the tips of the bundle of hair. Smoothness during spreading and a light or heavy sensation during spreading were evaluated on the basis of the evaluation criteria described below.

5 points: The sample spread well to the tips of hair, superior smoothness was exhibited, and a natural application sensation was provided.
4 points: The sample spread well to the tips of hair, and smoothness was exhibited, but a slight film-foaming sensation was exhibited.
3 points: A good spreading property was exhibited, but a remarkable film-forming sensation was exhibited. Alternatively, a good spreading property was exhibited, but a light feeling on touch was exhibited, and remarkable characteristics were not exhibited.
2 points: The sample spread to the tips of hair, but a slightly heavy sensation was exhibited and poor smoothness was exhibited.
1 point: A heavy sensation and poor spreading property were exhibited, in particular, roughness was exhibited at the tips of hair and smoothness was lacking.

(B) Smoothness in Running Water During Rinsing

The same operations as described in the aforementioned (A) were carried out, followed by rinsing the bundle of hair to which the sample had been applied, with warm running water. Rinsing was carried out by combing the bundle of hair with fingers 10 times, and the feeling on touch at that time was evaluated on the basis of the evaluation criteria described below.

5 points: Natural smoothness continued until the last 10^{th} rinsing operation. At the same time, a good coating sensation was also exhibited.
4 points: A smooth feeling on touch was totally exhibited, but a slight film-forming sensation was exhibited from the 8^{th} or 9^{th} rinsing operation.
3 points: Smoothness was exhibited until the 5^{th} rinsing operation, but a film-forming sensation was remarkably exhibited from the 6^{th} rinsing operation.
2 points: The sample was cleansed off until the 5^{th} rinsing operation, and the feeling on touch for conditioning weakly remained. Alternatively, poor smoothness was exhibited from the early rinsing operations, and a poor slipping sensation was exhibited at the second half of the rinsing operations.
1 point: Smoothness lacked from the early rinsing operation, and roughness and a frictional sensation were exhibited at the second half of the rinsing operations.

(C) Feeling on Touch During Drying with a Towel

The same operations as those described in the aforementioned (B) smoothness at the time of rinsing in running water were carried out. Subsequently, the wet bundle of hair was wrapped up with a towel to remove moisture. The feeling on touch of the moist bundle of hair (refreshing light smoothness sensation) was evaluated on the basis of the evaluation criteria described below.

5 points: A refreshing light and good smoothness sensation was exhibited over the bundle of hair including the tips of hair.
4 points: A refreshing smoothness sensation was exhibited as a whole.
3 points: Smoothness of the bundle of hair was exhibited, but is not light, and a slight slippery sensation was exhibited. Alternatively, slight smoothness was exhibited as a whole, but this was not remarkable.
2 points: A frictional sensation at the tips of hair was remarkably exhibited. Alternatively, poor smoothness was exhibited, and a heavy film-forming sensation was exhibited as a whole.
1 point: Poor smoothness was exhibited, and a frictional sensation was strongly exhibited.

(D) Conditioning Effects after Drying

The same operations as those described in the aforementioned (C) feeling on touch at the time of drying with a towel were carried out. Subsequently, the bundle of hair was completely dried with a drier, and conditioning effects (refreshing light combability with fingers) were evaluated on the basis of the evaluation criteria described below.

15 points: The bundle of hair possessed a refreshing light feeling on touch as a whole, and superior combability with fingers was exhibited.
12 points: A refreshing feeling on touch was exhibited as a whole, and good combability with fingers was exhibited over the bundle of hair.
9 points: Good combability with fingers was exhibited at almost all parts of the bundle of hair, but a heavy sensation was partially exhibited.
6 points: Poor smoothness tended to be exhibited as a whole, and slightly poor combability with fingers was exhibited.
3 points: Poor combability was clearly exhibited, and scratches or a frictional sensation was strongly exhibited.

TABLE 3

| | | | | | Comp. | Comp. |
|---|---|---|---|---|---|---|
| No. | Component | Ex. 1 | Ex. 2 | Ex. 3 | Ex. 4 | Ex. 5 |
| 1 | Cetanol | 5.6 | 5.6 | 5.6 | 5.6 | 5.6 |
| 2 | Stearyltrimonium | 1.5 | 1.5 | 1.5 | 1.5 | 1.5 |
| | chloride | | | | | |
| 3 | Behentrimonium chloride | 0.8 | 0.8 | 0.8 | 0.8 | 0.8 |
| 4 | Mineral oil | 1.0 | 1.0 | 1.0 | 1.0 | 1.0 |
| 5 | Decamethyl- | 2.0 | 2.0 | 2.0 | 2.0 | 2.0 |
| | cyclopentasiloxane | | | | | |
| 6 | Dimethylpoly-siloaxne, | — | — | — | 0.5 | 0.5 |
| | 2 cs | | | | | |
| 7 | Dimethylpoly-siloxane, | 1.0 | 1.0 | 1.0 | 1.0 | 1.0 |
| | 5,000 cs | | | | | |
| 8 | Phenyltrimethicone | 2.0 | 2.0 | 2.0 | 2.0 | 2.0 |
| 9 | Silicone Compound | 1.0 | — | — | — | — |
| | No. 1 | | | | | |
| 10 | Silicone Compound | — | 1.0 | — | — | — |
| | No. 2 | | | | | |
| 11 | Silicone Compound | — | — | 1.0 | — | — |
| | No. 3 | | | | | |
| 12 | Silicone Compound No. | — | — | — | 0.5 | — |
| | RE 1 | | | | | |
| 13 | Silicone Compound No. | — | — | — | — | 0.5 |
| | RE 2 | | | | | |
| 14 | Methylisothia-zolinone | 0.1 | 0.1 | 0.1 | 0.1 | 0.1 |
| 15 | EDTA-2Na | 0.1 | 0.1 | 0.1 | 0.1 | 0.1 |
| 16 | Purified water | 82.9 | 82.9 | 82.9 | 82.9 | 82.9 |
| Total number of parts | | 100.0 | 100.0 | 100.0 | 100.0 | 100.0 |
| Evaluation category | | Evaluation results (points) | | | | |
| WET | Sensation during use, | 5 | 4 | 5 | 3 | 3 |
| | at the time of applying | | | | | |
| | Smoothness during | 5 | 4 | 5 | 2 | 3 |
| | rinsing in running water | | | | | |
| | Feeling on touch during | 5 | 4 | 5 | 2 | 3 |
| | drying with a towel | | | | | |
| DRY | Conditioning effects | 15 | 12 | 15 | 3 | 9 |
| | after drying | | | | | |

| | | | | | |
|---|---|---|---|---|---|
| Total points | 30 | 24 | 30 | 10 | 18 |

Preparation Method of Hair Conditioner
(1) Components No. 1 to No. 13 were placed in a beaker with a volume of 200 mL, the mixture was heated and dissolved at 80° C. while stirring with a propeller mixer.
(2) Separately, components No. 15 and No. 16 were heated and dissolved at 80° C.
(3) The mixture obtained in the aforementioned (2) was added to the mixture obtained in the aforementioned (1) which was stirred, to emulsify the mixtures.
(4) The emulsion obtained in the aforementioned (3) was cooled while stirring, and component No. 14 was added thereto at 40° C. or less.

The hair conditioners of the present invention were superior with respect to conventional hair conditioners using a polyglycerol-modified silicone in view of both usage effects during WET, represented by (A) a sensation during use at the time of applying to hair (spreading smoothness and lightness or heaviness); (B) smoothness at the time of rinsing in running water; and (C) a feeling on touch at the time of drying with a towel (refreshing light smoothness sensation), and usage effects during DRY, represented by (D) conditioning effects after drying (refreshing light combability with fingers).

### Examples 4 and 5 and Comparative Examples 3 to 5

Shampoos were prepared with the compositions shown in Table 4, and evaluated in accordance with the evaluation criteria described below. The effects were quantified.

First, as cleansing effects, the following category was evaluated and pointed.
(A) Foam quality and foaming property: 5 stages of 1 to 5 points

As usage effects at the time of WET, the following categories were evaluated and pointed.
(B) Smoothness in the state of wet hair after rinsing hair: stages of 1 to 5 points
(C) Feeling on touch during drying hair with a towel (refreshing light and natural smoothness): 5 stages of 1 to 5 points

Subsequently, as usage effects at the time of DRY, the following category was evaluated and pointed.
(D) Conditioning effects after drying (refreshing light combability with fingers): 5 stages of 3, 6, 9, 12 and points

The total points at the time of WET are 15 points and the total points at the time of DRY are also 15 points. The results are also shown in Table 4. In the table, the numerical value described after each component indicates part(s) by weight (mass).

The methods for evaluating the aforementioned foam quality and foaming property, smoothness in the state of wet hair after rinsing hair, a feeling on touch during drying hair with a towel, and conditioning effects after drying, as well as evaluation criteria thereof are described below.

(A) Foam Quality and Foaming Property

Warm water was applied to hair to sufficiently contain moisture in hair. Subsequently, an appropriate amount (q.s.) of a shampoo composition of the present invention (the same amount as that which a panelist usually used in accordance with the length of hair of the panelist) was applied to hair with hands and shampooing was carried out by sufficiently applying the shampoo composition over the hair. At this time, the speed of foaming and foam quantity, as well as the fine texture of foam and foam uniformity were evaluated.

5 points: A superior foaming property was exhibited, superior foam quantity such as creamy and uniform foam with a fine texture was obtained, and a good feeling on touch was exhibited.
4 points: A good foaming property was exhibited, and good foam quantity such as uniform foam with a fine texture was obtained.
3 points: A normal foaming property was exhibited, and a normal foam texture and normal foam uniformity were obtained.
2 points: A normal foaming property was exhibited, but a slightly poor foam texture and slightly poor foam uniformity were obtained.
1 point: A poor foaming property and a coarse foam quality were exhibited, and foam quickly disappeared.

(B) Smoothness and a Shampooed Sensation in the State of Wet Hair after Rinsing Hair

The same operations as those described in the aforementioned (A) foam quality and foaming property were carried out. Subsequently, the shampooed hair was rinsed with warm running water by showering. Rinsing was carried out by rinsing off the shampoo by combining with fingers 10 times. The feeling on touch of the hair and a shampooed sensation (refreshing sensation by means of cleansing off hair) at the time of completion of the aforementioned rinsing operations times were evaluated.

5 points: Appropriate natural smoothness without an uncomfortable feeling on hair was exhibited, and the level of satisfaction of the shampooed sensation was also increased.
4 points: A slight film-foaming sensation was exhibited on the hair, but appropriate smoothness was exhibited, and the level of satisfaction of the shampooed sensation was also increased.
3 points: A film-forming sensation on the hair and poor smoothness were exhibited, but the level of satisfaction of the shampooed sensation was increased.
2 points: Poor combability with fingers and a frictional sensation were exhibited. As a result, the level of satisfaction of the shampooed sensation was slightly reduced.
1 point: A strong frictional sensation such as a sensation scratched by fingers was exhibited, and thereby, the level of satisfaction of the shampooed sensation was offset.

(C) Feeling on Touch During Drying with a Towel

The same operations as those described in the aforementioned (B) smoothness in the state of wet hair after rinsing hair were carried out. Subsequently, the wet hair was wrapped up with a towel to remove moisture. The feeling on touch of the moist hair (refreshing lightness and natural smoothness) was evaluated on the basis of the evaluation criteria described below.

5 points: Refreshing lightness and natural smoothness were exhibited without an uncomfortable sensation.
4 points: A slight film-forming sensation was imparted, but appropriate smoothness was exhibited over hair including the tips.
3 points: Appropriate smoothness was exhibited at almost all parts of the hair, but at the tips of hair a slightly frictional sensation was exhibited. Alternatively, slight smoothness was exhibited as a whole, but an unnatural film-forming sensation was also exhibited.
2 points: A heavy feeling on touch and reduced smoothness were exhibited as a whole. Alternatively, a frictional sensation at the tips of hair was remarkably exhibited.
1 point: A heavy feeling on touch and poor smoothness were exhibited, and a frictional sensation was strongly exhibited.

(D) Conditioning Effects after Drying

The same operations as those described in the aforementioned (C) feeling on touch at the time of drying with a towel were carried out. Subsequently, the hair was completely dried with a drier, and conditioning effects (refreshing light combability with fingers of the dried hair) were evaluated on the basis of the evaluation criteria described below.

15 points: The hair possessed a refreshing light sensation and natural combability with fingers was exhibited. No roughness of the tips of hair was exhibited.
12 points: The hair possessed a slightly heavy feeling on touch, but natural combability with fingers was exhibited. No roughness of the tips of hair was exhibited.
9 points: Normal combability with fingers was exhibited, but no roughness of the tips of hair was exhibited.
6 points: A slightly heavy feeling on touch was exhibited, and slightly poor combability with fingers was exhibited.
3 points: Rough hair was exhibited as a whole, poor combability was also exhibited, and a scratch sensation was exhibited.

TABLE 4

| | | | | Comp. | Comp. | Comp. |
|---|---|---|---|---|---|---|
| No. | Component | Ex. 4 | Ex. 5 | Ex. 4 | Ex. 5 | Ex. 6 |
| 1 | Sodium POE (2) lauryl ether sulfate (70% by weight (mass) aqueous | 17.86 | 17.86 | 17.86 | 17.86 | 17.86 |
| | solution) | | | | | |
| 2 | Cocamidopropylbetaine (30% by weight (mass) aqueous solution) | 8.33 | 8.33 | 8.33 | 8.33 | 8.33 |
| 3 | Cetanol | 0.5 | 0.5 | 0.5 | 0.5 | 0.5 |
| 4 | Cationated cellulose (2% by weight (mass) aqueous solution) | 25.0 | 25.0 | 25.0 | 25.0 | 25.0 |
| 5 | Cationated guar gum | 0.05 | 0.05 | 0.05 | 0.05 | 0.05 |
| 6 | Copolymer-type cationic polymer of dimethyldiallylammonium halide | 1.67 | 1.67 | 1.67 | 1.67 | 1.67 |
| | and acrylamide (9% by weight (mass) aqueous solution) | | | | | |
| 7 | Sodium benzoate | 0.3 | 0.3 | 0.3 | 0.3 | 0.3 |
| 8 | Glycol distearate | 1.0 | 1.0 | 1.0 | 1.0 | 1.0 |
| 9 | O/W emulsion*¹ obtained by emulsifying a mixture of Silicone | 4.0 | — | — | — | — |
| | Compound No. 1 and dimethylpolysiloxane (2 cs) | | | | | |
| 10 | O/W emulsion*¹ obtained by emulsifying a mixture of Silicone | — | 4.0 | — | — | — |
| | Compound No. 3 and dimethylpolysiloxane (2 cs) | | | | | |
| 11 | O/W emulsion*¹ for comparison obtained by emulsifying a mixture of | — | — | 4.0 | — | — |
| | Silicone Compound RE 1 and dimethylpolysiloxane (2 cs) | | | | | |
| 12 | O/W emulsion*¹ for comparison obtained by emulsitying a mixture of | — | — | — | 4.0 | — |
| | Silicone Compound RE 2 and dimethylpolysiloxane (2 cs) | | | | | |
| 13 | Citric acid | 0.05 | 0.05 | 0.05 | 0.05 | 0.05 |
| 14 | Purified water | 41.24 | 41.24 | 41.24 | 41.24 | 45.24 |
| Total number of parts | | 100.0 | 100.0 | 100.0 | 100.0 | 100.0 |
| Evaluation category | | Evaluation results (points) | | | | |
| WET | Foam quality and foaming property | 4 | 5 | 4 | 4 | 5 |
| | Smoothness of wet hair after rinsing | 5 | 5 | 1 | 2 | 1 |
| | Feeling on touch during drying with a towel | 5 | 5 | 1 | 2 | 1 |
| DRY | Conditioning effects atter drying | 15 | 15 | 6 | 6 | 3 |

| | | | | | |
|---|---|---|---|---|---|
| Total points | 29 | 30 | 12 | 14 | 10 |

Note
^{*}O/W emulsion comprising 60% by weight (mass) of a liquid obtained by mixing and uniforming a silicone compound (10 parts) and a dimethylpolysiloxane (2 cs) (90 parts), produced by emulsifying the formulation shown in Table 5 described below.

TABLE 5
Table 5

| | | Parts by |
|---|---|---|
| | Type of raw | weight |
| Name of raw material | material | (mass) |
| Mixture of silicone compound and | Oil agent | 60.0 |
| dimethylpolysiloxane (2 cs) (10:90) | | |
| POE (4) lauryl ether | Nonionic | 2.1 |
| | emulsifier | |
| POE (25) lauryl ether | Nonionic | 2.9 |
| | emulsifier | |
| Cetyltrimethylammonium chloride | Cationic | 0.5 |
| (30% by weight (mass) aqueous | emulsifier | |
| solution) | | |
| Sodium benzoate | Preservative | 0.5 |
| Citric acid | pH adjustor | 0.2 |
| Purified water | Water | 33.8 |

| | |
|---|---|
| Total | 100.0 |

Preparation Method for a Shampoo
(1) Component No. 1 to Component No. 3, Component No. 7, Component No. 8, and Component No. 14 were placed in a beaker with a volume of 200 mL. The mixture was stirred by means of a propeller mixer and completely dissolved at 70° C.
(2) Components No. 4 to No. 6 were added to the solution obtained in the aforementioned step (1) while the temperature thereof was maintained at 70° C., and the mixture were completely dissolved.
(3) The solution obtained in the aforementioned step (2) was cooled under stirring, and Component No. 9 to Component No. 12 were added thereto at 55° C.
(4) The mixture was further cooled to room temperature, and Component No. 13 was added thereto under stirring.

It was verified that the shampoos of the present invention were superior, as compared with comparative shampoos using other polyglycerol-modified silicones used in Comparative Experiments, in view of all categories of (A) foam quality and a foaming property; usage effects at the time of WET, represented by (B) smoothness and a shampooed sensation in the state of wet hair after rinsing hair, and (C) a feeling on touch during drying hair with a towel (refreshing lightness and natural smoothness); and usage effects at the time of DRY, represented by (D) conditioning effects after drying (refreshing light combability with fingers).

### Examples 6 and 7 and Comparative Examples 7 to 9

Hair creams (setting type) were prepared with the compositions shown in Table 6. Evaluation thereof was carried out on the basis of the evaluation criteria described below, and effects thereof were quantified.

First, as usage effects from applying to drying, the following categories were evaluated and pointed.
(A) Reduction of stickiness after applying to hair and until drying: 3 stages of 1, 3, and 5;
(B) Smoothness after applying to hair and until drying: 3 stages of 1, 3, and 5.

As usage effects after finishing, the following categories were evaluated and pointed.
(C) Retention ability of setting: 3 stages of 1, 3, and 5;
(D) Reduction of a rough sensation of hair of which setting

had been finished: 3 stages of 1, 3, and 5.

The total points during use are 10 points and the total points at the time of finishing hair-setting are also 10 points. The results are also shown in Table 6. In the table, the numerical value described after each component indicates part(s) by weight (mass).

The evaluation methods and evaluation criteria of the aforementioned reduction of stickiness after applying to hair to drying, smoothness after applying to hair to drying, retention ability of setting, and reduction of a rough sensation of set hair are described below.

(A) Reduction of Stickiness after Applying to Hair to Drying

A commercially available bundle of Chinese hair (manufactured by Beaulax Co., Ltd., 30 cm, 4 g) was washed with a 10% solution of sodium laureth sulfate. Subsequently, 1.0 g of a sample (hair cream) was put on the palm of a hand, and lightly spread thereon, followed by applying the sample from the roots to the tips of the bundle of hair. The style of the bundle of hair was adjusted, and stickiness was evaluated until the hair was dried.

5 points: No stickiness was felt from applying to drying.
3 points: No stickiness was felt at the time of applying, but stickiness was slightly felt at the time of drying. 1 point: Stickiness was slightly felt at the time of applying, and stickiness was clearly felt at the time of drying.

(B) Smoothness after Applying Until Drying

The same operations as described in the aforementioned (A) were carried out, a sample was applied to the bundle of hair, and style of the hair was adjusted with a comb. Smoothness until the bundle of hair was dried was evaluated.

5 points: Superior smooth combability was exhibited.
3 points: Smooth combability was normal.
1 point: Poor combability was exhibited with scratching.

(C) Retention Ability of Setting

A bundle of hair having a length of 25 cm and a weight of 2 g was moisturized with water, and 0.5 g of a sample was applied thereon. The bundle of hair was rolled on a rod having a diameter of 15 mm and naturally dried. After drying, the rod was removed from the curled bundle of hair. The curled bundle of hair was hung for one hour in a thermo-hygrostat chamber (28° C., 90% RH). Subsequently, the length of the curled hair was measured. Retention ability of setting was calculated in accordance with the following equation with the length (l₁) of curled hair immediately after the rod was removed from the hair and the length (l₂) of the hair which was allowed to stand for one hour, and evaluated.

Retention ability of setting={(25−l₂)/(25−l₁)}×100(%)

5 points: retention ability of setting=90 to 100%
3 points: retention ability of setting=67 to 89%
1 point: retention ability of setting=34 to 66%

(D) Reduction of a Rough Sensation of Set Hair

The feeling on touch of the bundle of hair which had been dried in the aforementioned (A) was evaluated on the basis of the evaluation criteria described below.

5 points: Rough and coarse hardness was not exhibited, natural smoothness was possessed, and a good styling sensation was obtained.
3 points: A rough and hard feeling on touch was slightly exhibited, but at the same time, a slightly smooth sensation was exhibited.
1 point: Rough and coarse hardness and a scratching sensation were exhibited.

TABLE 6

| | | | | Comp. | Comp. | Comp. |
|---|---|---|---|---|---|---|
| No. | Component | Ex. 6 | Ex. 7 | Ex. 8 | Ex. 9 | Ex. 10 |
| 1 | Carrageenan | 1.0 | 1.0 | 1.0 | 1.0 | 1.0 |
| 2 | POE (60) hardened castor oil | 1.0 | 1.0 | 1.0 | 1.0 | 1.0 |
| 3 | Carboxyvinyl polymer | 0.6 | 0.6 | 0.6 | 0.6 | 0.6 |
| 4 | Triethanolamine | q.s. | q.s. | q.s. | q.s. | q.s. |
| | | pH 7.5 | pH 7.5 | pH 7.5 | pH 7.5 | pH 7.5 |
| 5 | Glycerol | 2.0 | 2.0 | 2.0 | 2.0 | 2.0 |
| 6 | Perfume | q.s. | q.s. | q.s. | q.s. | q.s. |
| 7 | Octyl methoxycinnamate | 0.1 | 0.1 | 0.1 | 0.1 | 0.1 |
| 8 | Ethanol | 25.0 | 25.0 | 25.0 | 25.0 | 25.0 |
| 9 | Purified water | 58 | 58 | 58 | 58 | 63 |
| 10 | Amphoteric polymer: Copolymer of N-methacryloyl- | 3.0 | 3.0 | 3.0 | 3.0 | 3.0 |
| | oxydiethyl-N,N-dimethylaminoethyl-alpha-N- | | | | | |
| | methylcarboxybetaine and alkyl methacrylate ester | | | | | |
| 11 | Anionic polymer: Alkyl acrylate copolymer TEA (30% | 1.0 | 1.0 | 1.0 | 1.0 | 1.0 |
| | ethanol solution) | | | | | |
| 12 | O/W emulsion *2) obtained by emulsifying a mixture of | 8.0 | — | — | — | — |
| | Silicone Compound No. 1 and dimethylpolysiloxane (2 | | | | | |
| | cs) | | | | | |
| 13 | O/W emulsion *2) obtained by emulsifying a mixture of | — | 8.0 | — | — | — |
| | Silicone Compound No. 3 and dimethylpolysiloxane (2 | | | | | |
| | cs) | | | | | |
| 14 | O/W emulsion *2) obtained by emulsifying a mixture of | — | — | 8.0 | — | — |
| | Silicone Compound RE 1 for comparison and | | | | | |
| | dimethylpolysiloxane (2 cs) | | | | | |
| 15 | O/W emulsion *2) obtained by emulsifying a mixture of | — | — | — | 8.0 | — |
| | Silicone Compound RE 2 for comparison and | | | | | |
| | dimethylpolysiloxane (2 cs) | | | | | |
| Total number of parts | | 100 | 100 | 100 | 100 | 100 |
| Evaluation category | | Evaluation results (points) | | | | |
| During | Reduction of stickiness after applying and until | 5 | 5 | 3 | — *3) | 1 |
| use | drying | | | | | |
| | Smoothness after applying and until drying | 5 | 4 | 3 | — *3) | 1 |
| Finishing | Retention property of set hair | 5 | 5 | 3 | — *3) | 5 |
| | Reduction of rough sensation of finally styled | 5 | 5 | 3 | — *3) | 1 |
| | hair | | | | | |

| | | | | | |
|---|---|---|---|---|---|
| Total points | 20 | 19 | 12 | — *3) | 8 |

Note
*2) O/W emulsion which contains 30% by weight (mass) of a liquid produced by mixing a silicone compound (10 parts) with a dimethylpolysiloxane, 2 cs (90 parts), and uniforming the mixture, and which is produced by emulsifying the formulation shown by the following Table 7.
Note
*3) An emulsion could not be obtained with the composition shown in Table 7, and for this reason, evaluation for a hair cream by blending the composition was cancelled.

TABLE 7
Table 7

| | | Parts by |
|---|---|---|
| | Type of raw | weight |
| Name of raw material | material | (mass) |
| Mixture of silicone compound and | Oil agent | 30.0 |
| dimethylpolysiloxane (2 cs) (10:90) | | |
| Polyoxyethylene (4) alkyl (12-15) | Anionic | 1.7 |
| ether phosphoric acid | emulsifier | |
| POE (23) lauryl ether | Nonionic | 3.3 |
| | emulsifier | |
| Ethanol | Dispersant of | 2.0 |
| | paraben | |
| Propylparaben | Preservative | 0.05 |
| Methylparaben | Preservative | 0.15 |
| Triethanolamine | pH adjustor | 0.17 |
| Purified water | Water | 62.63 |

| | |
|---|---|
| Total | 100.0 |

Preparation Method for a Cream for Use on Hair (Setting Type)
(1) Half of Component No. 9 was placed in a beaker with a volume of 200 mL, and Components No. 1 to No. 3 and No. were added thereto. The mixture was stirred by means of a propeller mixer and uniformly dissolved.
(2) Components No. 4 and No. 6 to No. 8 were placed in another container, and they are uniformly dissolved.
(3) The remaining amount of Component No. 9 was gradually added to the solution obtained in the aforementioned step (2), which was being stirred, and thereby, a uniform dispersion was formed.
(4) Components No. 10 and No. 11 were gradually added to the dispersion obtained in the aforementioned step (3), which was being stirred. Thereby, a uniform viscous liquid was obtained.
(5) Components No. 12 to No. 15 were gradually added to the liquid obtained in the aforementioned step (4), which was being stirred. Thereby, a uniform cream was produced.

It was verified that the creams for use on hair (setting type) were superior, as compared with comparative creams for use on hair (setting type) using other polyglycerol-modified silicones used in Comparative Experiments, in view of both feeling on touch during use represented by (A) reduced stickiness after application and until drying, and (B) smoothness after application and until drying; and styling effects after finishing represented by (C) retention property of set hair, and (D) reduced a rough sensation after finishing setting.

Hereinafter, particular formulations of cosmetics for hair of the present invention are described as examples of the present invention. It should be understood that the present invention is not restricted thereto. In the series of Formulation Examples, in view of improvement of a feeling on touch to hair, Silicone Compound No. 1 (high polymerization 400) is the most preferred. For this reason, in Formulation Examples, Silicone Compound No. 1 is used. Therefore, it should be understood that Silicone Compound 5 used in Formulation Examples can be replaced with another sugar alcohol-modified silicone according to the present invention (such as the aforementioned Silicone Compound No. 2 or 3), and a mixture of two or more types of different sugar alcohol-modified silicones according to the present invention can also be used.

### Formulation Example 1

### Shampoo

The numerical value described after each component indicates part(s) by weight (mass).

(Components)

| | | |
|---|---|---|
| 1. | Purified water | remainder |
| 2. | Polyquaternium-10 | 0.3 |
| 3. | EDTA-2Na | 0.1 |
| 4. | Glycerol | 1.5 |
| 5. | Sodium laureth sulfate (27% aqueous solution) | 30.0 |
| 6. | Sodium laureth-6 carboxylate (24% aqueous solution) | 10.0 |
| 7. | Cocamidopropylbetaine, NaCl (30% aqueous solution) | 10.0 |
| 8. | Polyquaternium-7 | 0.27 |
| 9. | Preservatives | q.s. |
| 10. | Perfume | q.s. |
| 11. | Cocamido MEA | 2.0 |
| 12. | Emulsion of Silicone Compound No. 1(Note) | 0.5 |
| 13. | Citric acid | q.s. |

(Note):
O/W emulsion obtained by mixing Silicone Compound No. 1 and dimethylpolysiloxane (2 cSt) in a weight (mass) ratio of 1/9, and emulsifying the mixture so that the solid content is 30% by weight (mass).

Preparation Procedure

Step 1: Components 1 to 4 are heated, and subsequently, mixed and dissolved.
Step 2: Components 5 to 7 are added to the composition obtained in Step 1.
Step 3: The composition obtained in Step 2 is cooled, and components 8 to 12 are added thereto. Component 13 is added thereto, if necessary, to adjust the pH.

After Step 3, by further blending an emulsion such as a dimethylsilicone, a dimethylpolysiloxane (dimethiconol) of which both terminals are capped with dimethylsilanol groups, a phenyl-modified silicone, an amino-modified silicone, an aminopolyether-co-modified silicone or the like, an aqueous dispersion of silicone elastomer powders, and/or a water-soluble silicone oil such as a polyether-modified silicone or the like, or the like, the synergistic effects of respective components can be expected.

### Formulation Example 2

### Conditioner

The numerical value described after each component indicates part(s) by weight (mass).

(Components)

| | | |
|---|---|---|
| 1. | Stearyltrimonium chloride | 1.44 |
| 2. | Cetyl alcohol | 2.4 |
| 3. | Octyl dodecanol | 0.5 |
| 4. | Cetyl ethylhexanoate | 0.6 |
| 5. | Squalane | 0.2 |
| 6. | Purified water | remainder |
| 7. | Glycerol | 2.0 |
| 8. | Preservatives | q.s. |
| 9. | Perfume | q.s. |
| 10. | Emulsion of Silicone Compound No. 1(Note) | 3.0 |
| 11. | Citric acid | q.s. |

(Note):
O/W emulsion obtained by mixing Silicone Compound No. 1 and dimethylpolysiloxane (2 cSt) in a weight (mass) ratio of 1/9, and emulsifying the mixture so that the solid content is 30% by weight (mass).

Preparation Procedure

Step 1: Components 1 to 5 are heated, and subsequently, mixed and dissolved.
Step 2: Components 6 and 7 are heated, and subsequently, mixed and dissolved.
Step 3: The composition obtained in Step 2 is added to the composition obtained in Step 1 to emulsify the mixture.
Step 4: The composition obtained in Step 3 is cooled, and components 8 to 10 are added thereto. Component 11 is added thereto, if necessary.

After Step 4, by further blending an emulsion such as a dimethylsilicone, a dimethylpolysiloxane (dimethiconol) of which both terminals are capped with dimethylsilanol groups, a phenyl-modified silicone, an amino-modified silicone, an aminopolyether-co-modified silicone or the like, an aqueous dispersion of silicone elastomer powders, and/or a water-soluble silicone oil such as a polyether-modified silicone or the like, or the like, the synergistic effects of respective components can be expected.

### Formulation Example 3

### Hair Treatment, Rinse-in-Type

The numerical value described after each component indicates part(s) by weight (mass).

(Components)

| | | |
|---|---|---|
| 1. | Cetyl alcohol | 5.6 |
| 2. | Mineral oil | 1.0 |
| 3. | Stearyltrimonium chloride | 1.2 |
| 4. | Behentrimonium chloride | 0.64 |
| 5. | Cyclopentasiloxane | 2.0 |
| 6. | Dimethicone (2 cSt) | 1.0 |
| 7. | Dimethicone (5,000 cSt) | 1.0 |
| 8. | Phenylmethicone | 2.0 |
| 9. | Glycerol | 2.0 |
| 10. | EDTA-2Na | 0.1 |
| 11. | Purified water | remainder |
| 12. | Panthenol | 0.1 |
| 13. | Tocopherol | 0.04 |
| 14. | Lysine HCl | 0.02 |
| 15. | Glycine | 0.02 |
| 16. | Histidine | 0.02 |
| 17. | Silicone Compound No. 1 | 0.5 |
| 18. | Preservatives | q.s. |
| 19. | Perfume | q.s. |

Preparation Procedure

Step 1: Components 1 to 8 are heated, and subsequently, mixed and dissolved.
Step 2: Components 9 to 11 are heated, and subsequently, mixed and dissolved.
Step 3: The composition obtained in Step 2 is added to the composition obtained in Step 1 to emulsify the mixture.
Step 4: The composition obtained in Step 3 is cooled, and components 12 to 19 are added thereto.

In addition, in Step 1, by further adding an emulsion such as a dimethylpolysiloxane (dimethiconol) of which both terminals are capped with dimethylsilanol groups, an amino-modified silicone, an aminopolyether-co-modified silicone or the like, in addition to components 1 to 8, the synergistic effects of respective components can be expected.

### Formulation Example 4

### Hair Treatment, Leave-on-Type

The numerical value described after each component indicates part(s) by weight (mass).

(Components)

| | | |
|---|---|---|
| 1. | Cetyl alcohol | 4.0 |
| 2. | Mineral oil | 1.0 |
| 3. | Stearyltrimonium chloride | 1.0 |
| 4. | Behentrimonium chloride | 0.2 |
| 5. | Cyclopentasiloxane | 1.2 |
| 6. | Dimethicone (2 cSt) | 0.6 |
| 7. | Dimethicone (5,000 cSt) | 0.6 |
| 8. | Phenylmethicone | 1.2 |
| 10. | Glycerol | 2.0 |
| 11. | EDTA-2Na | 0.1 |
| 12. | Purified water | remainder |
| 13. | Panthenol | 0.1 |
| 14. | Tocopherol | 0.04 |
| 15. | Lysin HCl | 0.02 |
| 16. | Glycine | 0.02 |
| 17. | Histidine | 0.02 |
| 18. | Silicone Compound No. 1 | 0.3 |
| 19. | Preservatives | q.s. |
| 20. | Perfume | q.s. |

Preparation Procedure

Step 1: Components 1 to 8 are heated, and subsequently, mixed and dissolved.
Step 2: Components 9 to 11 are heated, and subsequently, mixed and dissolved.
Step 3: The composition obtained in Step 2 is added to the composition obtained in Step 1 to emulsify the mixture.
Step 4: The composition obtained in Step 3 is cooled, and components 12 to 20 are added thereto.

In addition, in Step 1, by further adding a dimethylpolysiloxane (dimethiconol) of which both terminals are capped with dimethylsilanol groups, an amino-modified silicone, an aminopolyether-co-modified silicone or the like, in addition to components 1 to 8, the synergistic effects of respective components can be expected.

### Formulation Example 5

### Hair Mist

The numerical value described after each component indicates part(s) by weight (mass).

(Components)

| | | |
|---|---|---|
| 1. | Purified water | remainder |
| 2. | Sorbitol | 0.6 |
| 3. | Creatine | 0.2 |
| 4. | Urea | 1.0 |
| 5. | 1,3-butylene glycol | 2.0 |
| 6. | Preservatives | q.s. |
| 7. | Ethanol | 15.0 |
| 8. | Glycereth-25 PCA isosteate | 0.5 |
| 9. | Perfume | q.s. |
| 10. | PEG/PPG-30/10 dimethicone, DPG (Note) | 1.0 |
| 11. | Silicone Compound No. 1 | 1.0 |
| 12. | Bisethoxydiglycol cyclohexanedicarboxylate | 2.0 |
| 13. | Hydroxypropyltrimonium starch chloride | 1.0 |

(Note):
BY 25-338, manufactured by Dow Corning Toray Co., Ltd.

Preparation Procedure

Step 1: Components 1 to 6 are mixed and dissolved.
Step 2: Components 7 to 10 are mixed and dissolved.
Step 3: The composition obtained in Step 2 is added to the composition obtained in Step 1 to solubilize.
Step 4: Components 11 to 13 are added to the composition obtained in Step 3, and the mixture is mixed and dissolved.

### Formulation Example 6

### Foam for Use on Hair

The numerical value described after each component indicates part(s) by weight (mass).

### Liquid

(Components)

| | | |
|---|---|---|
| 1. | Copolymer of polyvinylpyrrolidone and | 5.0 |
| | vinyl acetate | |
| 2. | Diethylsulfate salt of copolymer of | 0.5 |
| | vinylpyrrolidone and N,N-dimethylamino- | |
| | ethylmethacrylic acid | |
| 3. | Phenyltrimethicone | 2.0 |
| 4. | Silicone Compound No. 1 | 1.0 |
| 5. | Ethanol | 12.0 |
| 6. | Preservatives | q.s. |
| 7. | Perfume | q.s. |
| 8. | Purified water | remainder |

Formulation

| | | |
|---|---|---|
| 9. | Liquid | 95.0 |
| 10. | Liquid petroleum gas (LPG) | 5.0 |

Preparation Procedure

Step 1: Components 1 to 8 are mixed and dissolved.
Step 2: The composition (Liquid=Component 9) obtained in Step 1 is placed in a container (can), and a valve is loaded. Subsequently, Component 10 is placed therein.

In addition, in Step 1, a copolymer of acrylate and polytrimethylsiloxy methacrylate (such as FA 4001 CM (30% decamethylcyclopentasiloxane solution), manufactured by Dow Corning Toray Co., Ltd.) may be added as a film-forming agent, in addition to components 1 to 8.

### Formulation Example 7

### Hair Spray

The numerical value described after each component indicates part(s) by weight (mass).

### Liquid:

(Components)

| | | |
|---|---|---|
| 1. | Ethyl alcohol | remainder |
| 2. | Alkanolamine liquid of acrylic resin | 7.0 |
| | (active ingredient = 50%) | |
| 3. | Cetyl alcohol | 0.1 |
| 4. | Silicone Compound No. 1 | 0.5 |
| 5. | Perfume | q.s. |

Formulation

| | | |
|---|---|---|
| 6. | Liquid | 50.0 |
| 7. | Dimethyl ether | 50.0 |

Preparation Procedure

Step 1: Components 2 to 5 are added to component 1, and the mixture is mixed and dissolved.
Step 2: The composition obtained in Step 1 is filtered.
Step 3: The composition (Liquid=Component 6) obtained in Step 2 is placed in a container (can), and a valve is loaded. Subsequently, the container is charged with component 7.

### Formulation Example 8

### Hair Wax

The numerical value described after each component indicates part(s) by weight (mass).

(Components)

| | | |
|---|---|---|
| 1. | Diethylhexyl succinate | 10.0 |
| 2. | Squalane | 1.0 |
| 3. | Shear butter | 1.0 |
| 4. | Silicone Compound No. 1 | 2.0 |
| 5. | Candelilla wax | 5.5 |
| 6. | Microcrystalline wax | 6.0 |
| 7. | Carnauba wax | 6.0 |
| 8. | Ceteth-6 | 6.0 |
| 9. | Ceteth-10 | 6.0 |
| 10. | Glyceryl stearate (SE) soap impurities | 1.5 |
| 11. | Hydroxystearic acid | 4.5 |
| 12. | Purified water | remainder |
| 13. | 1,3-butylene glycol | 3.0 |
| 14. | Sodium hydroxide | q.s. |
| 15. | PEG-90M | q.s. |
| 16. | Preservatives | q.s. |

Preparation Procedure

Step 1: Components 1 to 11 are heated, and subsequently, mixed and dissolved.
Step 2: Components 12 to 14 are heated, and subsequently, mixed and dissolved.
Step 3: The composition obtained in Step 2 is added to the composition obtained in Step 1, and the mixture is emulsified.
Step 4: Components 15 and 16 are successively added to the composition obtained in Step 3.

### Formulation Example 9

### Cream for Use on Hair

The numerical value described after each component indicates part(s) by weight (mass).

(Components)

| | | |
|---|---|---|
| 1. | Vaseline | 4.0 |
| 2. | Cetyl ethylhexanoate | 3.0 |
| 3. | Silicone Compound No. 1 (Note) | 2.0 |
| 4. | Dimethicone (350 cSt) | 1.0 |
| 5. | PEG-40 hydrogenated castor oil | 1.0 |
| 6. | Polyacrylamide | 1.0 |
| 7. | Purified water | remainder |
| 8. | Glycerol | 3.0 |
| 9. | Hydroxyethylcellulose | 0.1 |
| 10. | Ethanol | 3.0 |
| 11. | Preservatives | q.s. |

(Note):
Decamethylcyclopentasioxane solution of Silicone Compound No. 5 (active ingredient = 10% by weight (mass)).

Preparation Procedure

Step 1: Components 1 to 5 are heated, and subsequently, mixed and dissolved.
Step 2: Components 6 to 9 are heated, and subsequently, mixed and dissolved.
Step 3: The composition obtained in Step 2 is added to the composition obtained in Step 1, and the mixture is emulsified.
Step 4: Components 10 and 11 are successively added to the composition obtained in Step 3.

In addition, in Step 1, by further adding a dimethylsilicone, a dimethylpolysiloxane (dimethiconol) of which both terminals are capped with dimethylsilanol groups, a phenyl-modified silicone, an amino-modified silicone, an aminopolyether-co-modified silicone or the like, in addition to components 1 to 5, the synergistic effects of respective components can be expected.

### Formulation Example 10

### Lotion for Use on Hair

The numerical value described after each component indicates part(s) by weight (mass).

(Components)

| | | |
|---|---|---|
| 1. | Carbomer | 0.4 |
| 2. | Hydroxyethylcellulose | 0.1 |
| 3. | PEG-6 | 1.5 |
| 4. | Purified water | remainder |
| 5. | Ethanol | 3.5 |
| 6. | PEG-40 hydrogenated castor oil | 0.5 |
| 7. | Trilaureth-4 phosphate | 0.1 |
| 8. | Cetyl ethylhexanoate | 2.0 |
| 9. | Emulsion of Silicone Compound No. 1(Note 1) | 1.2 |
| 10. | Emulsion of dimethicone (Note 2) | 2.5 |
| 11. | Preservatives | q.s. |
| 12. | Sodium hydroxide | q.s. |

(Note 1):
O/W emulsion obtained by mixing Silicone Compound No. 1 and dimethylpolysiloxane (2 cSt) in a weight (mass) ratio of 1/9, and emulsifying the mixture so that the solid content is 30% by weight (mass).
(Note 2):
FZ-4150 (active ingredient = 30% by weight (mass)), manufactured by Dow Corning Toray Co., Ltd.

Preparation Procedure

Step 1: Components 1 to 4 are heated, and subsequently, mixed and dissolved.
Step 2: Components 5 to 7 are heated, and subsequently, mixed and dissolved.
Step 3: The composition obtained in Step 2 is added to the composition obtained in Step 1, and the mixture is emulsified.
Step 4: Components 8 to 12 are added to the composition obtained in Step 3.

In addition, in Step 4, by adding an emulsion such as a dimethylsilicone, a dimethylpolysiloxane (dimethiconol) of which both terminals are capped with dimethylsilanol groups, a phenyl-modified silicone, an amino-modified silicone, an aminopolyether-co-modified silicone or the like, an aqueous dispersion of silicone elastomer powders, a water-soluble silicone oil such as a polyether-modified silicone or the like, or the like, in addition to components 8 to 12, the synergistic effects of respective components can be expected.

### Formulation Example 11

### Oil for Use on Hair

The numerical value described after each component indicates part(s) by weight (mass).

Preparation Procedure

(Components)

| | | |
|---|---|---|
| 1. | Cyclopentasiloxane solution of dimethicone (Note) | remainder |
| 2. | Silicone Compound No. 1 | 3.0 |
| 3. | Dimethicone (350 cSt) | 2.0 |
| 4. | Decamethylcyclopentasiloxane | 28.0 |

(Note):
BY11-003, manufactured by Dow Corning Toray Co., Ltd.

Step 1: Components 1 to 4 are appropriately heated, and subsequently, mixed and dissolved.

### Formulation Example 12

### Hair Color of Oxidation Type

The numerical value described after each component indicates part(s) by weight (mass).

### First Agent

(Components)

| | | |
|---|---|---|
| 1. | Steareth-2 | 3.0 |
| 2. | Steareth-21 | 2.0 |
| 3. | Stearyl PPG-15 | 5.0 |
| 4. | Cetostearyl alcohol | 4.0 |
| 5. | Behenyl alcohol | 2.0 |
| 6. | Silicone Compound No. 5 | 2.0 |
| 7. | Behenyltrimethylammonium chloride | 0.8 |
| 8. | Purified water | remainder |
| 9. | EDTA-2Na | 0.5 |
| 10. | Anhydrous sodium sulfite | 0.5 |
| 11. | Sodium ascorbate | 0.1 |
| 12. | 1,3-butylene glycol | 3.0 |
| 13. | p-phenylenediamine | 0.25 |
| 14. | p-aminophenol | 0.1 |
| 15. | m-aminophenol | 0.05 |
| 16. | Polyquaternium-39 | 0.3 |
| 17. | Ammonium hydrogen carbonate | 2.0 |
| 18. | Strong aqueous ammonia | 5.0 |

Preparation Procedure

Step 1: Components 1 to 7 are heated, and subsequently, mixed and dissolved.
Step 2: Components 8 to 15 are heated, and subsequently, mixed and dissolved.
Step 3: The composition obtained in Step 1 is added to the composition obtained in Step 2, and the mixture is emulsified.
Step 4: Components 16 to 18 are successively added to the composition obtained in Step 3.

Second Agent

(Components)

| | | |
|---|---|---|
| 1. | Cetostearyl alcohol | 4.5 |
| 2. | Sodium laurylsulfate | 0.5 |
| 3. | Preservatives | q.s. |
| 4. | Etidronic acid | 0.1 |
| 5. | Disodium hydrogen phosphate | 0.3 |
| 6. | Purified water | remainder |
| 7. | Hydrogen peroxide solution (35% aqueous solution) | 17.14 |
| 8. | Phosphoric acid | q.s. |

Preparation Procedure

Step 1: Component 1 is heated and dissolved.
Step 2: Components 2 to 6 are heated, and subsequently, mixed and dissolved.
Step 3: The component obtained in Step 1 is added to the composition obtained in Step 2, and the mixture is emulsified.
Step 4: The composition obtained in Step 3 is cooled. Component 7 is added thereto and Component 8 is added thereto, if necessary.

### Formulation Example 13

### Hair Manicure

The numerical value described after each component indicates part(s) by weight (mass).

(Components)

| | | |
|---|---|---|
| 1. | Black No. 401 | 0.4 |
| 2. | Violet No. 401 | 0.1 |
| 3. | Orange No. 205 | 0.3 |
| 4. | Benzyl alcohol | 5.0 |
| 5. | Citric acid | 0.5 |
| 6. | Hydroxyethylcellulose | 2.0 |
| 7. | Stearyltrimethylammonium chloride | 0.5 |
| 8. | PEG-40 hydrogenated castor oil | 0.5 |
| 9. | Silicone Compound No. 5 | 1.0 |
| 10. | Ethanol | 10.0 |
| 11. | Preservatives | q.s. |
| 12. | Perfume | q.s. |
| 13. | Purified water | remainder |
| 14. | Sodium citrate | q.s. |

Preparation Procedure

Step 1: Components 1 to 13 are mixed and dissolved.
Step 2: Component 14 is added to the composition obtained in Step 1, and thereby, the pH of the mixture is adjusted.

### Formulation Example 14

### Preparation for Permanent Waving

The numerical value described after each component indicates part(s) by weight (mass).

First Agent

(Components)

| | | |
|---|---|---|
| 1. | EDTA-2Na | 0.1 |
| 2. | Etidronic acid | 0.1 |
| 3. | Preservatives | q.s. |
| 4. | Purified water | remainder |
| 5. | PEG-40 hydrogenated castor oil | 0.6 |
| 6. | Perfume | 0.3 |
| 7. | Ammonium thioglycolate(50% aqueous solution) | 13.0 |
| 8. | Strong aqueous ammonia | 1.0 |
| 9. | Monoethanolamine | 1.2 |
| 10. | Ammonium hydrogen carbonate | 2.0 |
| 11. | Emulsion of Silicone Compound No. 5 (Note) | 0.5 |
| 12. | Phosphoric acid | q.s. |

Preparation Procedure

Step 1: Components 1 to 4 are appropriately heated, and subsequently, mixed and dissolved.
Step 2: Components 5 and 6 are heated, and subsequently, mixed and dissolved.
Step 3: The composition obtained in Step 2 is added to the composition obtained in Step 1.
Step 4: Components 7 to 11 are successively added to a composition obtained in Step 3. Component 12 is added thereto, if necessary.

Second Agent

(Components)

| | | |
|---|---|---|
| 1. | Polyquaternium-10 | 0.4 |
| 2. | EDTA-2Na | 0.1 |
| 3. | Preservatives | q.s. |
| 4. | Sodium dihydrogen phosphate | 0.05 |
| 5. | Disodium hydrogen phosphate | 0.5 |
| 6. | Purified water | remainder |
| 7. | Sodium bromate | 8.0 |
| 8. | pH adjustor | q.s. |

Note:
O/W emulsion obtained by mixing Silicone Compound No. 1 and dimethylpolysiloxane (2 cSt) in a weight (mass) ratio of 1/9, and emulsifying the mixture so that the solid content is 30% by weight (mass).

Preparation Procedure

Step 1: Components 1 to 6 are appropriately heated, and subsequently, mixed and dissolved.
Step 2: Component 7 is added to a composition obtained in Step 1. Component 8 is added thereto, if necessary.

1. A cosmetic for hair comprising (A) a sugar alcohol-modified silicone represented by the following general formula (1):
R¹ₐR²_{b}L¹_{c}Q_{d}SiO_{(4-a-b-c-d)/2} (1)
wherein
R¹ represents a monovalent organic group, with the proviso that R², L and Q are excluded therefrom;
R² represents a substituted or non-substituted, and linear or branched monovalent hydrocarbon group having 9 to 30 carbon atoms, or a linear organosiloxane group represented by the following general formula (2-1): 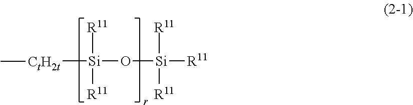
wherein R¹¹ is a substituted or non-substituted monovalent hydrocarbon group having
1 to 30 carbon atoms, a hydroxyl group or a hydrogen atom, and at least one R¹¹ is said monovalent hydrocarbon group; t is a number ranging from 2 to 10; and r is a number ranging from 1 to 500,
or represented by the following general formula (2-2): 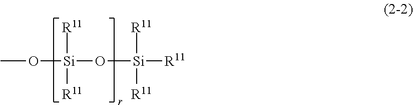
wherein R¹¹ and r are the same as defined above;
L¹ represents a silylalkyl group having a siloxane dendron structure, in the case of i=1, represented by the following general formula (3): 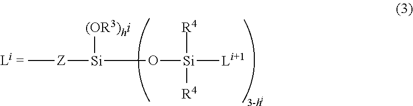
wherein
R³ represents a substituted or non-substituted, and linear or branched monovalent hydrocarbon group having 1 to 30 carbon atoms;
R⁴ independently represents an alkyl group having 1 to 6 carbon atoms or a phenyl group;
Z represents a divalent organic group;
i specifies the number of generations of said silylalkyl group, represented by Lⁱ, in the case in which the number of generations of said silylalkyl group, which is the number of repetitions of said silylalkyl group, is k, i is an integer ranging from 1 to k, and the number of generations k is an integer ranging from 1 to 10;
Lⁱ⁺¹ is said silylalkyl group in the case of i<k, and Lⁱ⁺¹ is R⁴ in the case of i=k; and
hⁱ is a number ranging from 0 to 3;
Q represents an organic group containing a sugar alcohol group; and
each of a, b, c and d is independently a number having the following range: 1.0≦a≦2.5, 0≦b≦1.5, 0≦c≦1.5, and 0.0001≦d≦1.5.

2. The cosmetic for hair according to claim 1, wherein in said general formula (1), the monovalent organic group, which is R¹, represents a substituted or non-substituted, and linear or branched monovalent hydrocarbon group having 1 to 8 carbon atoms, a polyoxyalkylene group represented by the following formula: —R⁵O(AO)ₙR⁶ wherein AO represents an oxyalkylene group having 2 to 4 carbon atoms; R⁵ represents a substituted or non-substituted, and linear or branched divalent hydrocarbon group having 3 to 5 carbon atoms; R⁶ represents a hydrogen atom, a substituted or non-substituted, and linear or branched monovalent hydrocarbon group having 1 to 24 carbon atoms, or a substituted or non-substituted, and linear or branched acyl group having 2 to 24 carbon atoms; and n=1 to 100, an alkoxy group, a hydroxyl group or a hydrogen atom, with the proviso that all R¹s do not represent a hydroxyl group, a hydrogen atom, said alkoxy group or said polyoxyalkylene group.

3. The cosmetic for hair according to claim 1, wherein in said general formula (1), Q is an organic group containing a sugar alcohol group represented by the following general formula (4-1): 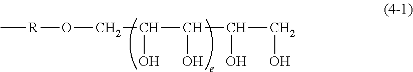
wherein R represents a divalent organic group; and e is 1 or 2,
or represented by the following general formula (4-2): 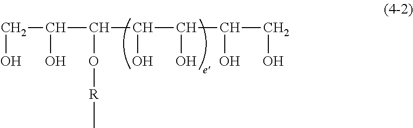
wherein R is the same as defined above; and e′ is 0 or 1.

4. The cosmetic for hair according to claim 1, wherein in said general formula (4-1) or (4-2), the divalent organic group, which is R, is a substituted or non-substituted, and linear or branched divalent hydrocarbon group having 3 to 5 carbon atoms.

5. The cosmetic for hair according to claim 1, wherein in said general formula (1), the silylalkyl group having a siloxane dendron structure, represented by L¹, is a functional group represented by the following general formula (3-1): 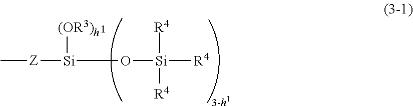
or represented by the following general formula (3-2): 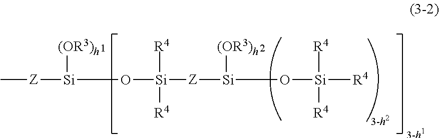
wherein R³, R⁴ and Z are the same as defined above; and
each of h¹ and h² is independently a number ranging from 0 to 3.

6. The cosmetic for hair according to claim 1, wherein said (A) sugar alcohol-modified silicone is represented by the following structural formula (1-1): 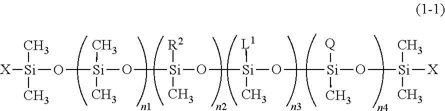
wherein
R², L¹ and Q are the same as defined above;
X is a group selected from the group consisting of a methyl group, R², L¹ and Q;
each of n1, n2, n3 and n4 is independently a number ranging from 0 to 2,000, and n1+n2+n3+n4 is a number ranging from 1 to 2,000, with the proviso that in the case of n4=0, at least one X is Q.

7. The cosmetic for hair according to claim 1, wherein said (A) sugar alcohol-modified silicone is represented by the following structural formula (1-1-1): 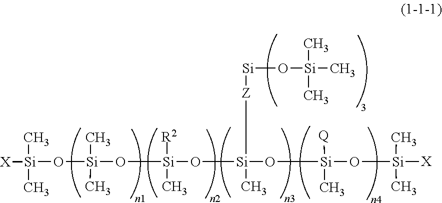
wherein R², Q, X, Z, n1, n2, n3 and n4 are the same as defined above,
or represented by the following structural formula (1-1-2): 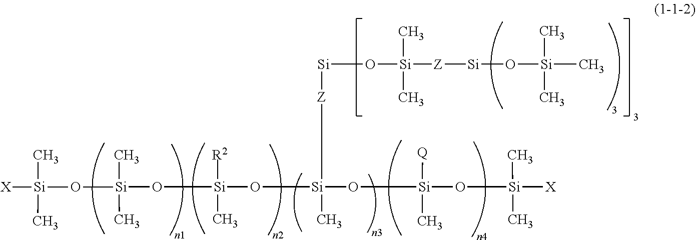
wherein R², Q, X, Z, n1, n2, n3, and n4 are the same as defined above.

8. The cosmetic for hair according to claim 1, wherein in said structural formula (1-1-1) or structural formula (1-1-2), Z is independently a group selected from divalent organic groups represented by the following general formulae:
—R⁷—
—R⁷—COO—R⁸—
—CO—R⁷—
—R⁷—COO—R⁸—
—R⁷—CONH—R⁸—
—R⁷—R⁸—
wherein
each R⁷ independently represents a substituted or non-substituted, and linear or branched, alkylene or alkenylene group having 2 to 22 carbon atoms or an arylene group having 6 to 22 carbon atoms;
R⁸ is a group selected from the group consisting of the following groups:

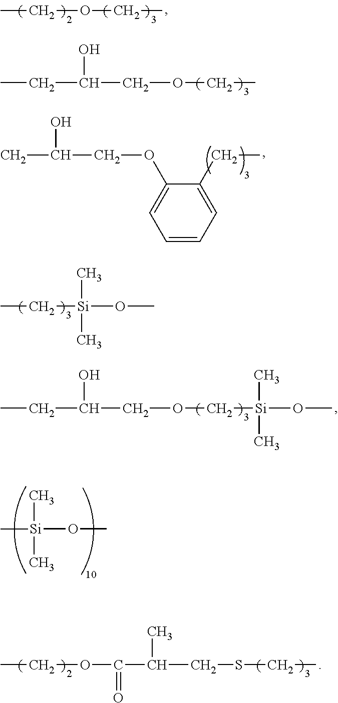

9. The cosmetic for hair according to claim 1, further comprising (B) an oil agent.

10. The cosmetic for hair according to claim 1, further comprising (C) a surfactant.

11. The cosmetic for hair according to claim 1, further comprising (D) a water-soluble polymer.

12. The cosmetic for hair according to claim 1, which is in the form of a cosmetic for cleansing hair, a cosmetic for conditioning hair, a cosmetic for styling hair, or a cosmetic for dyeing hair.

13. The cosmetic for cleansing hair according to claim 12, further comprising (C1) an anionic surfactant and (D1) a cationic water-soluble polymer.

14. The cosmetic for conditioning hair according to claim 12, further comprising (B2-1) a higher alcohol and (C2) a cationic surfactant.

15. The cosmetic for styling hair according to claim 12, which is in the form of a liquid, a cream, a solid, a paste, a gel, a mousse, or a spray.

16. The cosmetic for dyeing hair according to claim 12, further comprising (K) an oxidation hair-dyeing agent and/or (L) a direct dye.
